SEQUENCE LISTING (66 sequences)

SEQ ID NO: 1 - DNA, 384 bp, Mus musculus
gaggttcagctggagcagcctggggctgaactggtgaagcctggggcttcagtgaagttgtcctgcaaggcttctggctacaccttcaccagctactatatgtactgggtgaagcagaggcctggacaaggccttgagtggattgggggaattaatcctagcaatggtggtactaacttcaatgagaagttcaagaacaaggccacactgactgtagacaaatcctccagcacagcctacatgcaactcagcagcctgacatctgaggactctgcggtctattactgtacaagagagggtaacccctactatactatgaactactggggtcaaggaacctcagtcaccgtctcctcagccaaaacgacacccccatctgtctat

SEQ ID NO: 2 - protein, 128 aa, Mus musculus
EVQLEQPGAELVKPGASVKLSCKASGYTFTSYYMYWVKQRPGQGLEWIGGINPSNGGTNFNEKFKNKATLTVDKSSSTAYMQLSSLTSEDSAVYYCTREGNPYYTMNYWGQGTSVTVSSAKTTPPSVY

SEQ ID NO: 3 - DNA, 324 bp, Mus musculus
gatattgtgatgacacagactcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaagctggaaa

SEQ ID NO: 4 - protein, 108 aa, Mus musculus
DIVMTQTPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWK

SEQ ID NO: 5 - DNA, 384 bp, Mus musculus; identical to SEQ ID NO: 1

SEQ ID NO: 6 - protein, 128 aa, Mus musculus; identical to SEQ ID NO: 2

SEQ ID NO: 7 - DNA, 375 bp, Mus musculus
gatattgtgatgacccagactccatcctccctgagtgtgtcagcaggagagaaggtcactatgagctgcaagtccagtcagagtctgttaaacagtggaaatcaaaagacctacttggcctggtaccagcagaaaccagggcagcctcctaaactgttgatctacggggcatccactagggaatctggggtccctgatcgcttcacaggcagtggatctggaaccgatttcactcttaccatcagcagtgtgcaggctgaagacctggcagtttattactgtcagaatgatcatagttatccattcacgttcggctcggggacaaagttggaaataaaacgggctgatgctgcaccaactgtatccgcatgcacc

SEQ ID NO: 8 - protein, 122 aa, Mus musculus
DIVMTQTPSSLSVSAGEKVTMSCKSSQSLLNSGNQKTYLAWYQQKPGQPPKLLIYGASTRESGVPDRFTGSGSGTDFTLTISSVQAEDLAVYYCQNDHSYPFTFGSGTKLEIKRADAAPTVS

SEQ ID NO: 9 - DNA, 405 bp, Mus musculus
ctggagcagcctggggctgaactggtgaggcctggggcttcagtaatgctgtcctgcaaggcttctggctacatcttcaccacctactggatgcactggctgaagcagaggcctggacaaggccttgactggattggagagattagtcctagcaacggtcgttctaattacaataagaagttcaagagcaaggccacactgactgtagacaaatcctccagcacagcctacatgcaactcagcagcctgacatctgaggactctgcggtctattactgtgcacgattcgacgagggggacttctggggccaaggcaccactctcatagtctcctcagccaaaacaacagccccatcggtctatccactggcccctgtgtgtggagatacaactggctcctcggtg

SEQ ID NO: 10 - protein, 135 aa, Mus musculus
LEQPGAELVRPGASVMLSCKASGYIFTTYWMHWLKQRPGQGLDWIGEISPSNGRSNYNKKFKSKATLTVDKSSSTAYMQLSSLTSEDSAVYYCARFDEGDFWGQGTTLIVSSAKTTAPSVYPLAPVCGDTTGSSV

SEQ ID NO: 11 - DNA, 324 bp, Mus musculus; identical to SEQ ID NO: 3

SEQ ID NO: 12 - protein, 108 aa, Mus musculus; identical to SEQ ID NO: 4

SEQ ID NO: 13 - protein, 10 aa, Artificial Sequence
  Synthetic peptide
FLSRPTEKTI

SEQ ID NO: 14 - protein, 10 aa, Artificial Sequence
  Synthetic construct
KRDPCPHQVD

SEQ ID NO: 15 - protein, 9 aa, Artificial Sequence
  Synthetic peptide
LSAVYTCKR

SEQ ID NO: 16 - DNA, 24 bp, Artificial Sequence
  Synthetic oligonucleotide
ggctacaccttcaccagctactat

SEQ ID NO: 17 - DNA, 24 bp, Artificial Sequence
  Synthetic oligonucleotide
attaatcctagcaatggtggtact

SEQ ID NO: 18 - DNA, 36 bp, Artificial Sequence
  Synthetic oligonucleotide
acaagagagggtaacccctactatactatgaactac

SEQ ID NO: 19 - protein, 8 aa, Artificial Sequence
  Synthetic peptide
GYTFTSYY

SEQ ID NO: 20 - protein, 8 aa, Artificial Sequence
  Synthetic peptide
INPSNGGT

SEQ ID NO: 21 - protein, 12 aa, Artificial Sequence
  Synthetic peptide
TREGNPYYTMNY

SEQ ID NO: 22 - DNA, 24 bp, Artificial Sequence
  Synthetic oligonucleotide
ggctacatcttcaccacctactgg

SEQ ID NO: 23 - DNA, 24 bp, Artificial Sequence
  Synthetic oligonucleotide
attagtcctagcaacggtcgttct

SEQ ID NO: 24 - DNA, 24 bp, Artificial Sequence
  Synthetic oligonucleotide
gcacgattcgacgagggggacttc

SEQ ID NO: 25 - protein, 8 aa, Artificial Sequence
  Synthetic peptide
GYIFTTYW

SEQ ID NO: 26 - protein, 8 aa, Artificial Sequence
  Synthetic peptide
ISPSNGRS

SEQ ID NO: 27 - protein, 8 aa, Artificial Sequence
  Synthetic peptide
ARFDEGDF

SEQ ID NO: 28 - DNA, 36 bp, Artificial Sequence
  Synthetic oligonucleotide
cagagtctgttaaacagtggaaatcaaaagacctac

SEQ ID NO: 29 - DNA, 9 bp, Artificial Sequence
  Synthetic oligonucleotide
ggggcatcc

SEQ ID NO: 30 - DNA, 27 bp, Artificial Sequence
  Synthetic oligonucleotide
cagaatgatcatagttatccattcacg

SEQ ID NO: 31 - protein, 12 aa, Artificial Sequence
  Synthetic peptide
QSLLNSGNQKTY

SEQ ID NO: 32 - protein, 3 aa, Artificial Sequence
  Synthetic peptide
GAS

SEQ ID NO: 33 - protein, 9 aa, Artificial Sequence
  Synthetic peptide
QNDHSYPFT

SEQ ID NO: 34 - DNA, 30 bp, Artificial Sequence
  Synthetic oligonucleotide
aaaagtgtcagtacatctggctatagttat

SEQ ID NO: 35 - DNA, 9 bp, Artificial Sequence
  Synthetic oligonucleotide
cttgtatcc

SEQ ID NO: 36 - DNA, 23 bp, Artificial Sequence
  Synthetic oligonucleotide
cagcacattagggagcttacacg

SEQ ID NO: 37 - protein, 10 aa, Artificial Sequence
  Synthetic peptide
KSVSTSGYSY

SEQ ID NO: 38 - protein, 3 aa, Artificial Sequence
  Synthetic peptide
LVS

SEQ ID NO: 39 - protein, 7 aa, Artificial Sequence
  Synthetic peptide
QHIRELT

SEQ ID NO: 40 - DNA, 30 bp, Artificial Sequence; identical to SEQ ID NO: 34
  Synthetic oligonucleotide

SEQ ID NO: 41 - DNA, 9 bp, Artificial Sequence
  Synthetic oligonucleotide
cttgtatcc

SEQ ID NO: 42 - DNA, 24 bp, Artificial Sequence
  Synthetic oligonucleotide
cagcacattagggagcttacacgt

SEQ ID NO: 43 - protein, 10 aa, Artificial Sequence
  Synthetic peptide
KSVSTSGYSY

SEQ ID NO: 44 - protein, 3 aa, Artificial Sequence
  Synthetic peptide
LVS

SEQ ID NO: 45 - protein, 8 aa, Artificial Sequence
  Synthetic peptide
QHIRELTR

SEQ ID NO: 46 - DNA, 31 bp, Artificial Sequence
  Synthetic oligonucleotide
gagcctcttagaaagcgatggaaagacatat

SEQ ID NO: 47 - DNA, 9 bp, Artificial Sequence
  Synthetic oligonucleotide
ctggtgtct

SEQ ID NO: 48 - DNA, 27 bp, Artificial Sequence
  Synthetic oligonucleotide
tggcaaggtacacattttccgtggacg

SEQ ID NO: 49 - protein, 11 aa, Artificial Sequence
  Synthetic peptide
QSLLESDGKTY

SEQ ID NO: 50 - protein, 3 aa, Artificial Sequence
  Synthetic peptide
LVS

SEQ ID NO: 51 - protein, 9 aa, Artificial Sequence
  Synthetic peptide
WQGTHFPWT

SEQ ID NO: 52 - DNA, 357 bp, Artificial Sequence
  Synthetic oligonucleotide
gaggtccaactccagcagcctggggctgaactggtgaagcctggggcttcagtgaagttgtcctgcaaggcttctggctacaccttcaccagctactatatgtactgggtgaagcagaggcctggacaaggccttgagtggattgggggaattaatcctagcaatggtggtactaacttcaatgagaagttcaagaacaaggccacactgactgtagacaaatcctccagcacagcctacatgcaactcagcagcctgacatctgaggactctgcggtctattactgtacaagagagggtaacccctactatactatgaactactggggtcaaggaacctcagtcaccgtctcctca

SEQ ID NO: 53 - DNA, 345 bp, Artificial Sequence
  Synthetic oligonucleotide
gaggtccaactccagcaacctggggctgaactggtgaggcctggggcttcagtaatgctgtcctgcaaggcttctggctacatcttcaccacctactggatgcactggctgaagcagaggcctggacaaggccttgactggattggagagattagtcctagcaacggtcgttctaattacaataagaagttcaagagcaaggccacactgactgtagacaaatcctccagcacagcctacatgcaactcagcagcctgacatctgaggactctgcggtctattactgtgcacgattcgacgagggggacttctggggccaaggcaccactctcatagtctcctca

SEQ ID NO: 54 - DNA, 339 bp, Artificial Sequence
  Synthetic oligonucleotide
gacattgtgatgacgcagtctccatcctccctgagtgtgtcagcaggagagaaggtcactatgagctgcaagtccagtcagagtctgttaaacagtggaaatcaaaagacctacttggcctggtaccagcagaaaccagggcagcctcctaaactgttgatctacggggcatccactagggaatctggggtccctgatcgcttcacaggcagtggatctggaaccgatttcactcttaccatcagcagtgtgcaggctgaagacctggcagtttattactgtcagaatgatcatagttatccattcacgttcggctcggggacaaagttggaaataaaa

SEQ ID NO: 55 - DNA, 330 bp, Artificial Sequence
  Synthetic oligonucleotide
gacattgtgttgacacagtctcctgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggaggggggaccaagctggaaatcaaac

SEQ ID NO: 56 - DNA, 330 bp, Artificial Sequence
  Synthetic oligonucleotide
gatattgtgatgacccagtctcccgcttccttagctgtatctctggggcagagggccaccatctcatacagggccagcaaaagtgtcagtacatctggctatagttatatgcactggaaccaacagaaaccaggacagccacccagactcctcatctatcttgtatccaacctagaatctggggtccctgccaggttcagtggcagtgggtctgggacagacttcaccctcaacatccatcctgtggaggaggaggatgctgcaacctattactgtcagcacattagggagcttacacgttcggagggggggaccaagctggaaatcaaa

SEQ ID NO: 57 - DNA, 336 bp, Artificial Sequence
  Synthetic oligonucleotide
gacgttgtgatgacccagactccactcactttgtcggttaccattggacaaccagcctccatctcttgcaagtcaagtcagagcctcttagaaagcgatggaaagacatatttgaattggttgttacagaggccaggccagtctccaaagcgcctaatctatctggtgtctaaactggactctggagtccctgacaggttcactggcagtggatcagggacagatttcacactgaaaatcagcagagtggaggctgaggatttgggagtttattattgctggcaaggtacacattttccgtggacgttcggtggaggcaccaagctggaaatcaaa

SEQ ID NO: 58 - protein, 119 aa, Artificial Sequence
  Synthetic polypeptide
EVQLQQPGAELVKPGASVKLSCKASGYTFTSYYMYWVKQRPGQGLEWIGGINPSNGGTNFNEKFKNKATLTVDKSSSTAYMQLSSLTSEDSAVYYCTREGNPYYTMNYWGQGTSVTVSS

SEQ ID NO: 59 - protein, 115 aa, Artificial Sequence
  Synthetic polypeptide
EVQLQQPGAELVRPGASVMLSCKASGYIFTTYWMHWLKQRPGQGLDWIGEISPSNGRSNYNKKFKSKATLTVDKSSSTAYMQLSSLTSEDSAVYYCARFDEGDFWGQGTTLIVSS

SEQ ID NO: 60 - protein, 113 aa, Artificial Sequence
  Synthetic polypeptide
DIVMTQSPSSLSVSAGEKVTMSCKSSQSLLNSGNQKTYLAWYQQKPGQPPKLLIYGASTRESGVPDRFTGSGSGTDFTLTISSVQAEDLAVYYCQNDHSYPFTFGSGTKLEIK

SEQ ID NO: 61 - protein, 110 aa, Artificial Sequence
  Synthetic polypeptide
DIVLTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGPSWKSN

SEQ ID NO: 62 - protein, 110 aa, Artificial Sequence
  Synthetic polypeptide
DIVMTQSPASLAVSLGQRATISYRASKSVSTSGYSYMHWNQQKPGQPPRLLIYLVSNLESGVPARFSGSGSGTDFTLNIHPVEEEDAATYYCQHIRELTRSEGGTKLEIK

SEQ ID NO: 63 - protein, 112 aa, Artificial Sequence
  Synthetic polypeptide
DVVMTQTPLTLSVTIGQPASISCKSSQSLLESDGKTYLNWLLQRPGQSPKRLIYLVSKLDSGVPDRFTGSGSGTDFTLKISRVEAEDLGVYYCWQGTHFPWTFGGGTKLEIK

SEQ ID NO: 64 - protein, 8 aa, Artificial Sequence
  Synthetic construct
GYIFITTW

SEQ ID NO: 65 - protein, 9 aa, Artificial Sequence
  Synthetic construct
QNDYSYPFT

SEQ ID NO: 66 - protein, 113 aa, Artificial Sequence
  Synthetic construct
DIVMTQSPSSLSVSAGEKVTMSCKSSQSLLNSGNQKTYLAWYQQKPGQPPKLLIYGASTRESGVPDRFTGSGSGTDFTLTISSVQAEDLAVYYCQNDYSYPFTFGSGTKLEIK

(19) United States
(12) Patent Application Publication
(10) Pub. No.: US 2024/0076371 A1
(43) Pub. Date: Mar. 7, 2024

(54) COMPOSITIONS AND METHODS FOR TREATING OSTEOARTHRITIS, RHEUMATOID ARTHRITIS, AND JOINT AND TENDON DISORDERS

(71) Applicant: BOARD OF REGENTS, THE UNIVERSITY OF TEXAS SYSTEM, Austin, TX (US)

(72) Inventors: Jean X. Jiang, San Antonio, TX (US); Manuel A. Riquelme, San Antonio, TX (US); sumin Gu, San Antonio, TX (US); Rui Hua, San Antonio, TX (US)

(21) Appl. No.: 18/271,511

(22) PCT Filed: Jan. 13, 2022
(86) PCT No.: PCT/US2022/012267

§ 371 Date: Jul. 10, 2023

Related U.S. Application Data
(60) Provisional application No. 63/136,753, filed on Jan. 13, 2021.

(51) Int. Cl. C07K 16/28; A61P 19/02; A61P 29/00

(52) U.S. Cl. CPC C07K 16/28; A61P 19/02; A61P 29/00; A61K 2039/505; C07K 2317/24; C07K 2317/76

(57) ABSTRACT

Disclosed herein are antibodies useful that bind to connexin 43 hemichannels to inhibit or block channel opening. Also disclosed herein are methods for detecting or treating osteoarthritis, rheumatoid arthritis, and tendon disorders and injuries with antibodies that inhibit or block Cx43 hemichannel opening.

### CROSS REFERENCE TO RELATED APPLICATIONS

This application claims the benefit of the filing date of U.S. Provisional Application No. 63/136,753, filed on Jan. 13, 2021. The content of this earlier filed application is hereby incorporated by reference in its entirety.

### REFERENCE TO A SEQUENCE LISTING

The Sequence Listing submitted herein as a text file named “21105_0082P1_SL.txt,” created on Jan. 12, 2022, and having a size of 32,768 bytes is hereby incorporated by reference pursuant to 37 C.F.R. § 1.52(e)(5).

### BACKGROUND

Connexin hemichannels play important roles in the cell and tissue function, and abnormal function of connexin hemichannels may be involved various pathological conditions, such as those described herein. Thus, there remains a need for additional therapies for treating pathological conditions associated with hemichannels activity, as well as methods for identifying such therapies.

Osteoarthritis is the most common form of arthritis, affecting millions of people worldwide. Osteoarthritis can damage any joint and most commonly affects joints in hands, knees, hips and spine. Currently available therapy is associated with pain management. A need also exists for treating osteoarthritis.

### SUMMARY OF THE INVENTION

Disclosed herein are methods of treating or preventing osteoarthritis in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

Disclosed herein are methods of treating or preventing rheumatoid arthritis in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

Disclosed herein are methods of treating or preventing a tendon disorder or a tendon injury in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

Disclosed herein are methods of treating or preventing osteoarthritis in a subject, the methods comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

Disclosed herein are methods of treating or preventing rheumatoid arthritis in a subject, the methods comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

Disclosed herein are methods of treating or preventing a tendon disorder or a tendon injury in a subject, the methods comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

Disclosed herein are methods of treating or preventing osteoarthritis in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60.

Disclosed herein are method of treating or preventing rheumatoid arthritis in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60.

Disclosed herein are methods of treating or preventing a tendon disorder or a tendon injury in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60.

Disclosed herein are methods of reducing MMP13 or collagen X levels, the methods comprising administering to a subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

Disclosed herein are methods of reducing cartilage degradation in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

Disclosed herein are methods of reducing synovial inflammation in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

Disclosed herein are methods of reducing MMP13 or collagen X levels, the methods comprising administering to a subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

Disclosed herein are methods of reducing cartilage degradation in a subject, the methods comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

Disclosed herein are methods of reducing synovial inflammation in a subject, the methods comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

Disclosed herein are methods of reducing MMP13 or collagen X levels, the methods comprising administering to a subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60.

Disclosed herein are methods of reducing cartilage degradation in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60.

Disclosed herein are methods of reducing synovial inflammation in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60.

Disclosed herein are methods of treating or preventing subchondral bone sclerosis in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

Disclosed herein are methods of treating or preventing subchondral bone sclerosis in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 60.

Disclosed herein are methods of treating or preventing subchondral bone sclerosis in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60.

Disclosed herein are methods of reducing or ameliorating one or more symptoms of osteoarthritis in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58.

Disclosed herein are methods of reducing or ameliorating one or more symptoms of osteoarthritis in a subject, the methods comprising administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60.

Disclosed herein are methods of reducing or ameliorating one or more symptoms of osteoarthritis in a subject, the methods comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60.

Disclosed herein are recombinant connexin 43 (Cx43) hemichannel-binding antibodies or fragments thereof. In some aspects, the antibody comprises a first VH CDR corresponding to SEQ ID NO: 19 or a fragment thereof, a second VH CDR corresponding to SEQ ID NO: 20 or a fragment thereof, a third VH CDR corresponding to SEQ ID NO: 21 or a fragment thereof, a first VL CDR corresponding to SEQ ID NO: 31 or a fragment thereof, a second VL CDR corresponding to SEQ ID NO: 32 or a fragment thereof, and a third VL CDR corresponding to SEQ ID NO: 33 or a fragment thereof. In some aspects, the antibody or fragment thereof can be a humanized antibody. In some aspects, the antibody can be an IgG, IgM, IgA, IgD, IgE, or a genetically modified IgG class antibody comprising a first VH CDR corresponding to SEQ ID NO: 19, a second VH CDR corresponding to SEQ ID NO: 20, a third VH CDR corresponding to SEQ ID NO: 21, a first VL CDR corresponding to SEQ ID NO: 31, a second VL CDR corresponding to SEQ ID NO: 32, and a third VL CDR corresponding to SEQ ID NO: 33. In some aspects, the antibody can be an IgG class of antibody, wherein the IgG class antibody can be an IgG1, IgG2, IgG3, or IgG4 class antibody. In some aspects, the antibody comprises a VH amino acid sequence at least 90% identical to SEQ ID NO: 58 or a fragment thereof and/or a VL amino acid sequence at least 90% identical to SEQ ID NO: 60 or a fragment thereof. In some aspects, the antibody may comprise a VH amino acid sequence according to SEQ ID NO: 58 or a fragment thereof and/or a VL amino acid sequence according to SEQ ID NO: 60 or a fragment thereof.

Other aspects of the invention are discussed throughout this application. Any aspect discussed with respect to one aspect of the invention applies to other aspects of the invention as well and vice versa. Each aspect described herein is understood to be aspects of the invention that are applicable to other aspects of the invention. It is contemplated that any aspect discussed herein can be implemented with respect to any method or composition of the invention, and vice versa. Furthermore, compositions and kits of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-D show that M1 antibody treatment preserves cartilage integrity. FIG. 1A is a diagram of the timeline for surgical, injection and assessment procedures. FIG. 1B shows representative images of Safranin O/Fast Green histological staining of saline or M1 antibody-treated mice. Scale bar, 200 μm. FIG. 1C shows the Averaged Osteoarthritis Research Society International (OARSI) score of medial femoral condyle (MFC, upper panel) and medial tibial plateau (MTP, lower panel) at 8 weeks after DMM surgery. FIG. 1D shows the summed OARSI score of MTP and MFC through three-level sections of the joints. *P<0.05, **P<0.01, n=8-13 mice per group.

FIGS. 2A-B show that M1 antibody treatment decreased synovitis. FIG. 2A shows representative images of saline or Cx43 antibody-treated mice for synovitis scoring. M, meniscus; L, synovial lining cells. FIG. 2B shows the summarized degree of synovitis at 8 weeks after DMM surgery. *P<0.05, n=8-13 mice per group.

FIGS. 3A-B show that M1 antibody treatment reduced collagen X (Col X) levels in articular cartilage. FIG. 3A shows representative images of immunohistochemical stained ColX in the articular cartilage matrix of mice that underwent surgical DMM operations which were treated with saline or Cx43 antibody. Scale bar, 50 μm. FIG. 3B shows the quantification of the percentage of ColX positive chondrocytes. *P<0.05, n=5 mice per group.

FIGS. 4A-B show M1 antibody treatment reduced Matrix Metalloproteinase 13 (MMP13) Levels in articular cartilage. FIG. 4A shows representative images of immunohistochemical stained MMP13 in the articular cartilage matrix of mice that underwent surgical DMM operations which were treated with saline or Cx43 antibody. Scale bar, 50 μm. FIG. 4B shows the quantification of the percentage of MMP13 positive chondrocytes. *P<0.05, n=5 mice per group.

FIGS. 5A-E show that inhibition of Cx43 hemichannels protect against subchondral bone sclerosis. FIG. 5A shows representative two-dimensional microcomputed tomography (μCT) images of the medial and lateral subchondral bone compartments of the tibial plateau. Analysis of bone volume fraction (BV/TV) (FIG. 5B), trabecular thickness (Tb.Th) (FIG. 5C), trabecular spacing (Tb.Sp) (FIG. 5D), and bone mineral density (BMD) (FIG. 5E) of subchondral trabecular bone showed decreased subchondral bone sclerosis in DMM mice after Cx43 antibody treatment. *P<0.05; **P<0.01; ***P<0.001. n=6-8 mice per group.

FIGS. 6A-C show that M1 antibody treatment ameliorated osteoarthritis associated pain symptoms (Von Frey Filament Test). Mechanical sensitivity behavior assessments were performed at 4 weeks (FIG. 6B) and 8 weeks after surgery (FIGS. 6A and 6C). Withdrawal thresholds in the hind paw were measured by Von Frey filament test. *P<0.05, n=9-20 mice per group.

FIGS. 7A-F show that M1 antibody treatment ameliorated osteoarthritis associated pain symptoms (open field test). Open field behavior assessments were performed on mice at 4 weeks (FIGS. 7B and 7E) and 8 weeks after surgery (FIGS. 7A, 7C, 7D and 7F). FIGS. 7A-C show the total distance travelled and FIGS. 7D-F show total rest time during the 10 minutes testing period were analyzed. *P<0.05, n=9-20 mice per group.

FIGS. 8A-B show that M1 antibody primarily binds to the synovium. FIG. 8A shows a diagram of timeline for in vivo antibody binding assessment procedures. FIG. 8B shows frozen tissue sections prepared and immuno-labeled with Alexa Fluor 594-conjugated anti-human IgG secondary antibody. Scale Bar, 100 μm.

FIGS. 9A-B show that M1 antibody co-localized with synovial macrophages. FIG. 9A shows sections immuno-labeled with the macrophage marker CD68, and Cx43 antibody was co-localized with CD68 in the synovium region. Scale Bar, 10 μm. FIG. 9B shows an enlarged view of the white boxes from A (right panel). Scale Bar, 5 μm.

FIGS. 10A-B show M1 antibody co-localized with synovial fibroblasts. FIG. 10A shows tissue sections of immuno-labeling with the fibroblast marker Periostin, and M1 antibody was co-localized with Periostin in the synovium region. Scale Bar, 10 μm. FIG. 10B shows an enlarged view of the white boxes from FIG. 10A (right panel). Scale Bar, 5 μm.

FIGS. 11A-C show that M1 antibody delivered to synovium inhibits the opening of hemichannels after DMM surgery. FIG. 11A shows that mice treated with saline or M1 antibody at 30 mins after DMM surgery. Tail vein injection of Evans Blue was performed at two weeks after the treatment. Frozen sections were prepared and the hemichannel dye uptake was evaluated by fluorescence microscopy. FIG. 11B shows the ratio of Evans Blue intensity from the synovium region of right knee (with DMM surgery) and left knee (no surgery) was quantified by the NIH ImageJ Software. Scale Bar, 50 μm. **P<0.01, n=4 mice per group. FIG. 11C shows frozen sections prepared from saline-treated mice were immuno-labeled with the Cx43 antibody. Scale Bar, 50 μm.

FIGS. 12A-C show improved cartilage integrity with a single M1 antibody injection. FIG. 12A shows a diagram of timeline for surgical, injection and assessment procedures. FIG. 12B shows representative images of Safranin O/Fast Green histological staining of saline or M1 antibody-treated mice. Scale bar, 200 μm. FIG. 12C shows the Averaged Osteoarthritis Research Society International (OARSI) score of medial femoral condyle (MFC, upper panel) and medial tibial plateau (MTP, lower panel) at 8 weeks after DMM surgery. *P<0.05, **P<0.01, n=3-7 mice per group.

FIGS. 13A-B show the reduction of MMP13 level in articular cartilage with a single antibody injection. FIG. 13A shows representative images of immunohistochemical stained MMP13 in the articular cartilage matrix of mice that underwent surgical DMM operations which were treated with saline or M1 antibody. FIG. 13B shows the quantification of the staining intensity of MMP13 in chondrocytes. *P<0.05, n=5-6 mice per group.

FIGS. 14A-B show mitigation of pain related behaviors with a single M1 antibody injection. FIG. 14A shows that withdrawal thresholds in the hind paw were measured by Von Frey filament test at 8 weeks after surgery. FIG. 14B shows open field behavior assessments were performed at 8 weeks after surgery. Total distance travelled (left panel) and total rest time (right panel) during the 10 minutes testing period were analyzed. *P<0.05, n=6-8 mice per group.

FIG. 15 shows a proposed model of osteoarthritis. Osteoarthritis is a disease that affects all joint tissues, including the progressive degeneration of the articular cartilage, subchondral bone remodeling, osteophyte formation and synovial inflammation. The activated synovial cells in the inflamed synovium produce catabolic and proinflammatory mediators that lead to excess production of the proteolytic enzymes responsible for cartilage breakdown. The synovium of knee joints comprises macrophages and fibroblasts, which express Cx43. The administration of the M1 antibody blocks the hemichannel opening and release of inflammatory cytokines, and thus, mitigates the cartilage break down, subchondral bone sclerosis and pain symptoms.

FIGS. 16A-C show that delayed anti-Cx43 hemichannel antibody (M1 antibody) treatment decreases synovitis. FIG. 16A shows a timeline for surgical, injection and assessment procedures. FIG. 16B shows representative images of saline or anti-Cx43 hemichannel antibody (M1 antibody)-treated mice for synovitis scoring. FIG. 16C shows the degree of synovitis (synovitis score) at 8 weeks after DMM surgery. *P<0.05, n=6 mice per group.

FIGS. 17A-B show that the anti-Cx43 hemichannel antibody (M1 antibody) blocked Cx43 hemichannel opening in RAW264.7 and SW982 cells. FIG. 17A shows mouse macrophage RAW264.7 cells were treated with LPS and Cx43E2 antibody or CBX (100 μM). The level of EtBr dye uptake was determined and quantified by fluorescence microcopy and NIH Image J software. B) Human synovial fibroblast SW982 cells were treated with IL1β and Cx43E2 antibody or CBX (100 μM). The level of EtBr dye uptake was determined and quantified by fluorescence microcopy and NIH Image J software. Data shown are mean±SEM. ****, P<0.0001.

FIG. 18 shows that the anti-Cx43 hemichannel antibody (M1 antibody) inhibited LPS-induced inflammatory gene expression in RAW264.7 cells. Mouse macrophage RAW264.7 cells were pre-incubated with or without the Cx43E2 antibody for 30 min followed by incubation with 0.1 μg/ml LPS for 4 hrs. RNA extracts were prepared and subjected to qRT-PCR analysis for COX2, Adamts4, MMP3/13 and IL6 mRNA expression. *P<0.05, **P<0.01.

FIG. 19 shows that the anti-Cx43 hemichannel antibody (M1 antibody) inhibited IL1β-induced inflammatory gene expression in SW982 cells. Human synovial fibroblast SW982 cells were pre-incubated with or without the Cx43E2 antibody for 30 min followed by incubation with 1 ng/ml IL1β for 14 hrs. RNA extracts were prepared and subjected to qRT-PCR analysis for COX2, Adamts4/5, MMP3/13 and NOS2 mRNA expression. *P<0.05, **P<0.01.

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present disclosure is not entitled to antedate such publication by virtue of prior disclosures. Further, the dates of publication provided herein can be different from the actual publication dates, which can require independent confirmation.

### Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise.

The use of the word “a” or “an” when used in conjunction with the term “comprising” in the claims and/or the specification may mean “one,” but it is also consistent with the meaning of “one or more,” “at least one,” and “one or more than one.”

“Optional” or “optionally” means that the subsequently described event, circumstance, or material may or may not occur or be present, and that the description includes instances where the event, circumstance, or material occurs or is present and instances where it does not occur or is not present.

The word “or” as used herein means any one member of a particular list and also includes any combination of members of that list. The use of the term “or” in the claims is used to mean “and/or” unless explicitly indicated to refer to alternatives only or the alternatives are mutually exclusive, although the disclosure supports a definition that refers to only alternatives and “and/or.”

Throughout this application, the term “about” is used to indicate that a value includes the standard deviation of error for the device or method being employed to determine the value.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

“Inhibit,” “inhibiting” and “inhibition” mean to diminish or decrease an activity, level, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in some aspects, the inhibition or reduction can be a 10, 20, 30, 40, 50, 60, 70, 80, 90, 100%, or any amount of reduction in between as compared to native or control levels. In some aspects, the inhibition or reduction is 10-20, 20-30, 30-40, 40-50, 50-60, 60-70, 70-80, 80-90, or 90-100% as compared to native or control levels. In some aspects, the inhibition or reduction is 0-25, 25-50, 50-75, or 75-100% as compared to native or control levels.

“Modulate”, “modulating” and “modulation” as used herein mean a change in activity or function or number. The change may be an increase or a decrease, an enhancement or an inhibition of the activity, function or number.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in some aspects, the increase or promotion can be a 10, 20, 30, 40, 50, 60, 70, 80, 90, 100%, or more, or any amount of promotion in between compared to native or control levels. In some aspects, the increase or promotion is 10-20, 20-30, 30-40, 40-50, 50-60, 60-70, 70-80, 80-90, or 90-100% as compared to native or control levels. In some aspects, the increase or promotion is 0-25, 25-50, 50-75, or 75-100%, or more, such as 200, 300, 500, or 1000% more as compared to native or control levels. In some aspects, the increase or promotion can be greater than 100 percent as compared to native or control levels, such as 100, 150, 200, 250, 300, 350, 400, 450, 500% or more as compared to the native or control levels.

“Treatment” and “treating” refer to administration or application of a therapeutic agent (e.g., an anti-Cx43 antibody described herein) to a subject or performance of a procedure or modality on a subject for the purpose of obtaining a therapeutic benefit of a disease or health-related condition. For example, a treatment may include administration of a pharmaceutically effective amount of an antibody or fragment thereof that inhibits or blocks the opening of the Cx43 hemichannel.

As used herein, the term “treating” refers to partially or completely alleviating, ameliorating, relieving, delaying onset of, inhibiting or slowing progression of, reducing severity of, and/or reducing incidence of one or more symptoms or features of a particular disease, disorder, and/or condition. Treatment can be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition. For example, the disease, disorder, and/or condition can be osteoarthritis, rheumatoid arthritis, and tendon and joint disorders and injuries.

As used herein, the term “subject” refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In some aspects, a subject is a mammal. In another aspect, a subject is a human. The term does not denote a particular age or sex. Thus, adult, child, adolescent and newborn subjects, as well as fetuses, whether male or female, are intended to be covered.

As used herein, the term “patient” refers to a subject afflicted with a condition, disease or disorder. The term “patient” includes human and veterinary subjects. In some aspects of the disclosed methods, the “patient” has been diagnosed with osteoarthritis, rheumatoid arthritis, and tendon and joint disorders and injuries. In some aspects of the disclosed methods, the “patient” has been diagnosed with a need for treatment (e.g. treatment for osteoarthritis, rheumatoid arthritis, and tendon and joint disorders and injuries), such as, for example, prior to the administering step.

As used herein the terms “amino acid” and “amino acid identity” refers to one of the 20 naturally occurring amino acids or any non-natural analogues that may be in any of the antibodies, variants, or fragments disclosed. Thus “amino acid” as used herein means both naturally occurring and synthetic amino acids. For example, homophenylalanine, citrulline and norleucine are considered amino acids for the purposes of the invention. “Amino acid” also includes amino acid residues such as proline and hydroxyproline. The side chain may be in either the (R) or the (S) configuration. In some aspects, the amino acids are in the (S) or L-configuration. If non-naturally occurring side chains are used, non-amino acid substituents may be used, for example to prevent or retard in vivo degradation.

The term “fragment” can refer to a portion (e.g., at least 5, 10, 25, 50, 100, 125, 150, 200, 250, 300, 350, 400 or 500, etc. amino acids or nucleic acids) of a protein or nucleic acid molecule that is substantially identical to a reference protein or nucleic acid and retains the biological activity of the reference. In some aspects, the fragment or portion retains at least 50%, 75%, 80%, 85%, 90%, 95% or 99% of the biological activity of the reference protein or nucleic acid described herein. Further, a fragment of a referenced peptide can be a continuous or contiguous portion of the referenced polypeptide (e.g., a fragment of a peptide that is ten amino acids long can be any 2-9 contiguous residues within that peptide).

A “variant” can mean a difference in some way from the reference sequence other than just a simple deletion of an N- and/or C-terminal amino acid residue or residues. Where the variant includes a substitution of an amino acid residue, the substitution can be considered conservative or non-conservative. Conservative substitutions are those within the following groups: Ser, Thr, and Cys; Leu, Ile, and Val; Glu and Asp; Lys and Arg; Phe, Tyr, and Trp; and Gln, Asn, Glu, Asp, and His. Variants can include at least one substitution and/or at least one addition, there may also be at least one deletion. Variants can also include one or more non-naturally occurring residues. For example, they may include selenocysteine (e.g., seleno-L-cysteine) at any position, including in the place of cysteine. Many other “unnatural” amino acid substitutes are known in the art and are available from commercial sources. Examples of non-naturally occurring amino acids include D-amino acids, amino acid residues having an acetylaminomethyl group attached to a sulfur atom of a cysteine, a pegylated amino acid, and omega amino acids of the formula NH₂(CH₂)ₙCOOH wherein n is 2-6 neutral, nonpolar amino acids, such as sarcosine, t-butyl alanine, t-butyl glycine, N-methyl isoleucine, and norleucine. Phenylglycine may substitute for Trp, Tyr, or Phe; citrulline and methionine sulfoxide are neutral nonpolar, cysteic acid is acidic, and ornithine is basic. Proline may be substituted with hydroxyproline and retain the conformation conferring properties of proline.

Substitutional variants typically contain the exchange of one amino acid for another at one or more sites within the protein, and may be designed to modulate one or more properties of the polypeptide, with or without the loss of other functions or properties. Substitutions may be conservative, that is, one amino acid is replaced with one of similar shape and charge. Conservative substitutions are well known in the art and include, for example, the changes of: alanine to serine; arginine to lysine; asparagine to glutamine or histidine; aspartate to glutamate; cysteine to serine; glutamine to asparagine; glutamate to aspartate; glycine to proline; histidine to asparagine or glutamine; isoleucine to leucine or valine; leucine to valine or isoleucine; lysine to arginine; methionine to leucine or isoleucine; phenylalanine to tyrosine, leucine or methionine; serine to threonine; threonine to serine; tryptophan to tyrosine; tyrosine to tryptophan or phenylalanine; and valine to isoleucine or leucine. Alternatively, substitutions may be non-conservative such that a function or activity of the polypeptide is affected. Non-conservative changes typically involve substituting a residue with one that is chemically dissimilar, such as a polar or charged amino acid for a nonpolar or uncharged amino acid, and vice versa.

A “single-chain variable fragment (scFv)” means a protein comprising the variable regions of the heavy and light chains of an antibody. A scFv can be a fusion protein comprising a variable heavy chain, a linker, and a variable light chain. In some aspects, the linker can be a short, flexible fragment that can be about 8 to 20 amino acids in length. For example, (G4S)ₙ can be used (n=1, 2, 3 or 4).

The term “monoclonal antibody” (monoclonal antibody) refers to an antibody, or population of like antibodies, obtained from a population of substantially homogeneous antibodies, and is not to be construed as requiring production of the antibody by any particular method, including but not limited to, monoclonal antibodies can be made by the hybridoma method first described by Kohler and Milstein (Nature, 256: 495-497, 1975), or by recombinant DNA methods.

The term “chimeric antibody” (or “chimeric immunoglobulin”) refers to a molecule comprising a heavy and/or light chain which is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (Cabilly et al. (1984), infra; Morrison et al., Proc. Natl. Acad. Sci. U.S.A. 81:6851).

The term “humanized antibody” refers to forms of antibodies that contain sequences from non-human (e.g., murine) antibodies as well as human antibodies. A humanized antibody can include conservative amino acid substitutions or non-natural residues from the same or different species that do not significantly alter its binding and/or biologic activity. Such antibodies are chimeric antibodies that contain minimal sequence derived from non-human immunoglobulins. For the most part, humanized antibodies are human immunoglobulins (recipient antibody) in which residues from a complementary-determining region (CDR) of the recipient are replaced by residues from a CDR of a non-human species (donor antibody) such as mouse, rat, camel, bovine, goat, or rabbit having the desired properties. Furthermore, humanized antibodies can comprise residues that are found neither in the recipient antibody nor in the imported CDR or framework sequences. These modifications are made to further refine and maximize antibody performance. Thus, in general, a humanized antibody can comprise all or substantially all of at least one, and in one aspect two, variable domains, in which all or substantially all of the hypervariable loops correspond to those of a non-human immunoglobulin and all or substantially all of the FR regions are those of a human immunoglobulin sequence. The humanized antibody optionally also can comprise at least a portion of an immunoglobulin constant region (Fc), or that of a human immunoglobulin (see, e.g., Cabilly et al., U.S. Pat. No. 4,816,567; Cabilly et al., European Patent No. 0,125,023 B1; Boss et al., U.S. Pat. No. 4,816,397; Boss et al., European Patent No. 0,120,694 B1; Neuberger, M. S. et al., WO 86/01533; Neuberger, M. S. et al., European Patent No. 0,194,276 B1; Winter, U.S. Pat. No. 5,225,539; Winter, European Patent No. 0,239,400 B1; Padlan, E. A. et al., European Patent Application No. 0,519,596 A1; Queen et al. (1989) Proc. Natl. Acad. Sci. USA, Vol 86:10029-10033).

As used herein, the term “M1H” refers to an antibody that was cloned from hybridoma clones. “M1” refers to hybridoma monoclonal 1, and “H” refers to the variable heavy chain.

As used herein, the term “M1M7K” refers to the variable light chain identified from hybridoma clones M1 and M7.

As used herein, the term “antigen” is a molecule capable of being bound by an antibody or T-cell receptor. In some aspects, binding moieties other than antibodies can be engineered to specifically bind to an antigen, e.g., aptamers, avimers, and the like.

The term “antibody” or “immunoglobulin” is used to include intact antibodies and binding fragments/segments thereof. As used herein, the term “antibody” is intended to refer broadly to any immunologic binding agent, such as IgG, IgM, IgA, IgD, IgE, and genetically modified IgG as well as polypeptides comprising antibody CDR domains that retain antigen binding activity. The antibody may be selected from the group consisting of a chimeric antibody, an affinity matured antibody, a polyclonal antibody, a monoclonal antibody, a humanized antibody, a human antibody, or an antigen-binding antibody fragment or a natural or synthetic ligand. Typically, fragments compete with the intact antibody from which they were derived for specific binding to an antigen. Fragments include separate heavy chains, light chains, Fab, Fab′ F(ab′)2, Fabc, and Fv. Fragments/segments are produced by recombinant DNA techniques, or by enzymatic or chemical separation of intact immunoglobulins. The term “antibody” also includes one or more immunoglobulin chains that are chemically conjugated to, or expressed as, fusion proteins with other proteins. The term “antibody” also includes bispecific antibodies. A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai and Lachmann, Clin Exp Immunol 79:315-21, 1990; Kostelny et al., J. Immunol. 148:1547-53, 1992.

The term “antibody” can include five different classes of human immunoglobulins, namely IgG, IgA, IgM, IgD, and IgE. In some aspects, the disclosed antibodies can be an IgG class of antibody which can be classified into the 4 subclasses of IgG1, IgG2, IgG3, and IgG4. In some aspects, the disclosed antibodies can be an IgA class of antibody which, can be classified into the 2 subclasses of IgA1 and IgA2. The basic structure of immunoglobulin is made up of 2 homologous L chains (light chains) and 2 homologous H chains (heavy chains). The immunoglobulin class and subclass are determined by H chains. In some aspects, the antibody or antibodies or variants or fragments thereof can be an IgG4.

While antigen-binding specificity is maintained, antibody stability of IgG4 can be improved. In some aspects, the antibody can be improved, for example, by substituting arginine (R) of IgG4 with glutamic acid (E), phenylalanine (F), isoleucine (I), asparagine (N), glutamine (Q), serine (S), valine (V), tryptophan (W), tyrosine (Y), lysine (K), threonine (T), methionine (M), or leucine (L).

The term “isolated” can refer to a nucleic acid or polypeptide that is substantially free of cellular material, bacterial material, viral material, or culture medium (when produced by recombinant DNA techniques) of their source of origin, or chemical precursors or other chemicals (when chemically synthesized). Moreover, an isolated compound refers to one that can be administered to a subject as an isolated compound; in other words, the compound may not simply be considered “isolated” if it is adhered to a column or embedded in an agarose gel. Moreover, an “isolated nucleic acid fragment” or “isolated peptide” is a nucleic acid or protein fragment that is not naturally occurring as a fragment and/or is not typically in the functional state.

Moieties of the invention, such as antibodies, antibody fragments, polypeptides, peptides, antigens, or immunogens, may be conjugated or linked covalently or noncovalently to other moieties such as adjuvants, proteins, peptides, supports, fluorescence moieties, or labels. The term “conjugate” or “immunoconjugate” is broadly used to define the operative association of one moiety with another agent and is not intended to refer solely to any type of operative association, and is particularly not limited to chemical “conjugation.”

The term “providing” is used according to its ordinary meaning “to supply or furnish for use.” In some aspects, the protein is provided directly by administering the protein, while in other aspects, the protein is effectively provided by administering a nucleic acid that encodes the protein. In certain aspects the invention contemplates compositions comprising various combinations of nucleic acid, antigens, peptides, and/or epitopes.

The phrase “specifically binds” or “specifically immunoreactive” to a target refers to a binding reaction that is determinative of the presence of the molecule in the presence of a heterogeneous population of other biologics. Thus, under designated immunoassay conditions, a specified molecule binds preferentially to a particular target and does not bind in a significant amount to other biologics present in the sample. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane, Antibodies: A Laboratory Manual, Cold Spring Harbor Press, 1988, for a description of immunoassay formats and conditions that can be used to determine specific immunoreactivity.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Various cells are able to communicate with each other and with the extracellular environment through hemichannels and gap junctions formed by the protein connexin. Connexin proteins are ubiquitously expressed throughout the body. Six connexin proteins make up one hemichannel, and two hemichannels make up one gap junction channel. Gap junctions are a cluster of channels that are located in the plasma membrane between adjoining cells and they mediate intercellular communication. Hemichannels are a separate entity from gap junction channels. Hemichannels permit the exchange of molecules between the intracellular compartments and the extracellular environment.

Osteoarthritis (OA) is a joint disease characterized by progressive degeneration of articular cartilage, subchondral bone remodeling and synovial inflammation, which causes pain and limited mobility. Connexin 43 (Cx43) acts as a homeostatic regulator in the musculoskeletal system and its expression is increased in the cartilage and synovial tissue of OA patients. Cx43 likely contributes to the production of catabolic and inflammatory factors and exacerbates joint destruction. Cx43 forms hemichannels (HCs) that mediate the release of small molecules, such as ATP and prostaglandin E2 (PGE2), into the extracellular environment. The open probability of HCs is low under physiological conditions; their activation is regulated by multiple factors, including proinflammatory cytokines. Described herein are antibodies (e.g., M1 antibody) that specifically inhibit the activity of Cx43 HCs.

Antibodies

Disclosed herein are anti-connexin 43 antibodies (also referred to herein as “anti-Cx43 antibodies” or “anti-CX43 antibodies”). Disclosed herein are anti-connexin 43 antibodies that can inhibit or block the opening hemichannels, and in particular, Cx43 hemichannels. Disclosed herein are anti-connexin 43 antibodies that can activate or stimulate the opening hemichannels, and in particular, Cx43 hemichannels.

Application No. PCT/US2017/019605 (WO 2017-147561) provides examples of anti-Cx43 antibodies, CDR sequences, heavy chain and light chain sequences, nucleic acid sequences that encode said antibodies and the epitope sequences that said antibodies bind that can be used in the disclosed methods, which is hereby incorporated by reference for teaching the same.

Anti-Cx43 antibodies were generated and clones were identified that produced Cx43-binding antibodies. Anti-Cx43 antibodies were generated and clones were identified that produced Cx43-binding antibodies. Disclosed herein are anti-Cx43 antibodies comprising CDR sequences shown in the Tables below along with the pairing for each of the characterized antibodies. Examples of both DNA and amino acids for anti-Cx43 antibody sequences are also shown in the Tables below along with the correct pairing for each of the characterized antibodies. Examples of anti-Cx43 antibodies, include, but are not limited to M1 and M2 antibodies. The M1 antibody inhibits or blocks the opening of a Cx43 hemichannel. The M2 antibody activates, stimulates and/or enhances the opening of a Cx43 hemichannel.

As used herein, “M1 antibody” refers to an antibody comprising a variable heavy chain comprising a sequence having the sequence set forth in SEQ ID NO: 58; and a variable light chain comprising the sequence set forth in SEQ ID NO: 60.

As used herein, “M2 antibody” refers to an antibody comprising a variable heavy chain comprising a sequence having the sequence set forth in SEQ ID NO: 58; and a variable light chain comprising the sequence set forth in SEQ ID NO: 63.

Also disclosed are humanized anti-Cx43 antibodies. For example, disclosed herein are humanized M1 and M2 antibodies.

TABLE 1
Pairing of heavy chain and light chain for
two functional anti-Cx43 antibodies.

| Antibody Name | Heavy chain | Light chain |
|---|---|---|
| M1 (HmAb1) | M1H | M1K1 or M1K1a |
| M2 (HmAb2) | M1H | M1M7K |

TABLE 2
Sequence of antibody chains of anti-Cx43 antibodies from
the hybridomas generated.

| mAb | CDR-1 | CDR-2 | CDR-3 |
|---|---|---|---|
| M1H | ggctacaccttcaccagcta | attaatcctagcaatggtggt | acaagagagggtaaccccta |
| | ctat | act | ctatactatgaactac |
| | (SEQ ID NO: 16) | (SEQ ID NO: 17 | (SEQ ID NO: 18) |
| | GYTFTSYY | INPSNGGT | TREGNPYYTMNY |
| | (SEQ ID NO: 19) | (SEQ ID NO: 20) | (SEQ ID NO: 21) |
| M7H | ggctacatcttcaccaccta | attagtcctagcaacggtcg | gcacgattcgacgaggggga |
| | ctgg | ttct | cttc |
| | (SEQ ID NO: 22) | (SEQ ID NO: 23) | (SEQ ID NO: 24) |
| | GYIFTTYW | ISPSNGRS | ARFDEGDF |
| | (SEQ ID NO: 25) | (SEQ ID NO: 26) | (SEQ ID NO: 27) |
| M7Ha | GYIFITTW | ISPSNGRS | ARFDEGDF |
| | (SEQ ID NO: 64) | (SEQ ID NO: 26) | (SEQ ID NO: 27) |
| M1K1 | cagagtctgttaaacagtgg | ggggcatcc | cagaatgatcatagttatccatt |
| | aaatcaaaagacctac | (SEQ ID NO: 29) | cacg |
| | (SEQ ID NO: 28) | | (SEQ ID NO: 30) |
| | QSLLNSGNQKTY | GAS | QNDHSYPFT |
| | (SEQ ID NO: 31) | (SEQ ID NO: 32) | (SEQ ID NO: 33) |
| M1K1a | QSLLNSGNQKTY | GAS | QNDYSYPFT |
| | (SEQ ID NO: 31) | (SEQ ID NO: 32) | (SEQ ID NO: 65) |
| M1K2 | aaaagtgtcagtacatctgg | cttgtatcc | cagcacattagggagcttaca |
| | ctatagttat | (SEQ ID NO: 35) | cg |
| | (SEQ ID NO: 34) | | (SEQ ID NO: 36) |
| | KSVSTSGYSY | LVS | QHIRELT |
| | (SEQ ID NO: 37) | (SEQ ID NO: 38) | (SEQ ID NO: 39) |
| M2K | aaaagtgtcagtacatctgg | cttgtatcc | cagcacattagggagcttaca |
| | ctatagttat | (SEQ ID NO: 41) | cgt |
| | (SEQ ID NO: 40) | | (SEQ ID NO: 42) |
| | KSVSTSGYSY | LVS | QHIRELTR |
| | (SEQ ID NO: 43) | (SEQ ID NO: 44) | (SEQ ID NO: 45) |
| M1M7K | gagcctcttagaaagcgat | ctggtgtct | tggcaaggtacacattttccgt |
| | ggaaagacatat | (SEQ ID NO: 47) | ggacg |
| | (SEQ ID NO: 46) | | (SEQ ID NO: 48) |
| | QSLLESDGKTY | LVS | WQGTHFPWT |
| | (SEQ ID NO: 49) | (SEQ ID NO: 50) | (SEQ ID NO: 51) |

Cloned variable domains are shown in the charts below.

TABLE 3
DNA sequences of portions of the disclosed
anti-Cx43 antibodies. Variable heavy chain
(bold) and variable light chain (underlined).

| | | SEQ |
|---|---|---|
| | | ID |
| mAb | Sequence | NO: |
| M1H | GAGGTCCAACTCCAGCAGCCTGGGGCTGAACTGGTG | 52 |
| | AAGCCTGGGGCTTCAGTGAAGTTGTCCTGCAAGGCT | |
| | TCTGGCTACACCTTCACCAGCTACTATATGTACTG | |
| | GGTGAAGCAGAGGCCTGGACAAGGCCTTGAGTGGA | |
| | TTGGGGGAATTAATCCTAGCAATGGTGGTACTAAC | |
| | TTCAATGAGAAGTTCAAGAACAAGGCCACACTGACT | |
| | GTAGACAAATCCTCCAGCACAGCCTACATGCAACTC | |
| | AGCAGCCTGACATCTGAGGACTCTGCGGTCTATTAC | |
| | TGTACAAGAGAGGGTAACCCCTACTATACTATGA | |
| | ACTACTGGGGTCAAGGAACCTCAGTCACCGTCT | |
| | CCTCA | |
| M7H | GAGGTCCAACTCCAGCAACCTGGGGCTGAACTGGTG | 53 |
| | AGGCCTGGGGCTTCAGTAATGCTGTCCTGCAAGGCT | |
| | TCTGGCTACATCTTCACCACCTACTGGATGCACTG | |
| | GCTGAAGCAGAGGCCTGGACAAGGCCTTGACTGGAT | |
| | TGGAGAGATTAGTCCTAGCAACGGTCGTTCTAATT | |
| | ACAATAAGAAGTTCAAGAGCAAGGCCACACTGACTG | |
| | TAGACAAATCCTCCAGCACAGCCTACATGCAACTCAG | |
| | CAGCCTGACATCTGAGGACTCTGCGGTCTATTACTGT | |
| | GCACGATTCGACGAGGGGGACTTCTGGGGCCAAGG | |
| | CACCACTCTCATAGTCTCCTCA | |
| M1K1 | GACATTGTGATGACGCAGTCTCCATCCTCCCTGAGTG | 54 |
| | TGTCAGCAGGAGAGAAGGTCACTATGAGCTGCAAGT | |
| | CCAGTCAGAGTCTGTTAAACAGTGGAAATCAAAAGA | |
| | CCTACTTGGCCTGGTACCAGCAGAAACCAGGGCAGC | |
| | CTCCTAAACTGTTGATCTACGGGGCATCCACTAGGGA | |
| | ATCTGGGGTCCCTGATCGCTTCACAGGCAGTGGATCT | |
| | GGAACCGATTTCACTCTTACCATCAGCAGTGTGCAGG | |
| | CTGAAGACCTGGCAGTTTATTACTGTCAGAATGATCA | |
| | TAGTTATCCATTCACGTTCGGCTCGGGGACAAAGTTG | |
| | GAAATAAAA | |
| M1K2 | GACATTGTGTTGACACAGTCTCCTGCTTCCTTAGCTGT | 55 |
| | ATCTCTGGGGCAGAGGGCCACCATCTCATACAGGGCC | |
| | AGCAAAAGTGTCAGTACATCTGGCTATAGTTATATGC | |
| | ACTGGAACCAACAGAAACCAGGACAGCCACCCAGAC | |
| | TCCTCATCTATCTTGTATCCAACCTAGAATCTGGGGTC | |
| | CCTGCCAGGTTCAGTGGCAGTGGGTCTGGGACAGACT | |
| | TCACCCTCAACATCCATCCTGTGGAGGAGGAGGATGC | |
| | TGCAACCTATTACTGTCAGCACATTAGGGAGCTTACA | |
| | CGTTCGGAGGGGGGACCAAGCTGGAAATCAAAC | |
| M2K | GATATTGTGATGACCCAGTCTCCCGCTTCCTTAGC | 56 |
| | TGTATCTCTGGGGCAGAGGGCCACCATCTCATACA | |
| | GGGCCAGCAAAAGTGTCAGTACATCTGGCTATAGTTAT | |
| | ATGCACTGGAACCAACAGAAACCAGGACAGCCACCCA | |
| | GACTCCTCATCTATCTTGTATCCAACCTAGAATCTGGG | |
| | GTCCCTGCCAGGTTCAGTGGCAGTGGGTCTGGGACAG | |
| | ACTTCACCCTCAACATCCATCCTGTGGAGGAGGAGGA | |
| | TGCTGCAACCTATTACTGTCAGCACATTAGGGAGCT | |
| | TACACGTTCGGAGGGGGGGACCAAGCTGGAAATCA | |
| | AA | |
| M1M7K | GACGTTGTGATGACCCAGACTCCACTCACTTTGTCGG | 57 |
| | TTACCATTGGACAACCAGCCTCCATCTCTTGCAAGTCA | |
| | AGTCAGAGCCTCTTAGAAAGCGATGGAAAGACATATT | |
| | TGAATTGGTTGTTACAGAGGCCAGGCCAGTCTCCAAA | |
| | GCGCCTAATCTATCTGGTGTCTAAACTGGACTCTGGAG | |
| | TCCCTGACAGGTTCACTGGCAGTGGATCAGGGACAGA | |
| | TTTCACACTGAAAATCAGCAGAGTGGAGGCTGAGGAT | |
| | TTGGGAGTTTATTATTGCTGGCAAGGTACACATTTTCC | |
| | GTGGACGTTCGGTGGAGGCACCAAGCTGGAAATCAAA | |

Additional DNA sequences of portions of anti-Cx43 antibodies can be one or more the DNA sequences of portions of the disclosed anti-Cx43 antibodies disclosed in PCT/US2017/019605 (WO 2017/147561) which is hereby incorporated by references for its teaching of DNA sequences of portions of anti-Cx43 antibodies.

TABLE 4
Amino acid sequences of portions of the disclosed
anti-Cx43 antibodies. Variable heavy chain (bold)
and variable light chain (underlined).

| mAb | Sequence | SEQ ID NO: |
|---|---|---|
| M1H | EVQLQQPGAELVKPGASVKLSCKASGYTFTSYYMYWVKQR | 58 |
| | PGQGLEWIGGINPSNGGTNFNEKFKNKATLTVDKSSSTAYM | |
| | QLSSLTSEDSAVYYCTREGNPYYTMNYWGQGTSVTVSS | |
| M7H | EVQLQQPGAELVRPGASVMLSCKASGYIFTTYWMHWLKQR | 59 |
| | PGQGLDWIGEISPSNGRSNYNKKFKSKATLTVDKSSSTAYM | |
| | QLSSLTSEDSAVYYCARFDEGDFWGQGTTLIVSS | |
| M1K1 | DIVMTQSPSSLSVSAGEKVTMSCKSSQSLLNSGNQKTYLAW | 60 |
| | YQQKPGQPPKLLIYGASTRESGVPDRFTGSGSGTDFTLTISSV | |
| | QAEDLAVYYCQNDHSYPFTFGSGTKLEIK | |
| M1K2 | DIVLTQSPASLAVSLGQRATISYRASKSVSTSGYSYMHWNQ | 61 |
| | QKPGQPPRLLIYLVSNLESGVPARFSGSGSGTDFTLNIHPVE | |
| | EEDAATYYCQHIRELTRSEGGPSWKSN | |
| M2-K | DIVMTQSPASLAVSLGQRATISYRASKSVSTSGYSYMHWNQ | 62 |
| | QKPGQPPRLLIYLVSNLESGVPARFSGSGSGTDFTLNIHPVE | |
| | EEDAATYYCQHIRELTRSEGGTKLEIK | |
| M1M7-K | DVVMTQTPLTLSVTIGQPASISCKSSQSLLESDGKTYLNWLL | 63 |
| | QRPGQSPKRLIYLVSKLDSGVPDRFTGSGSGTDFTLKISRVE | |
| | AEDLGVYYCWQGTHFPWTFGGGTKLEIK | |
| M1K1a | DIVMTQSPSSLSVSAGEKVTMSCKSSQSLLNSGNQKTYLAW | 66 |
| | YQQKPGQPPKLLIYGASTRESGVPDRFTGSGSGTDFTLTISSV | |
| | QAEDLAVYYCQNDYSYPFTFGSGTKLEIK | |

Additional amino acid sequences of portions of anti-Cx43 antibodies anti-Cx43 antibodies can be one or more the amino acid sequences of portions of anti-Cx43 antibodies disclosed in PCT/US2017/019605 (WO 2017/147561) which is hereby incorporated by references for its teaching of amino acid sequences of portions of anti-Cx43 antibodies.

The term “CDR” as used herein refers to a Complementarity Determining Region of an antibody variable domain (e.g., of an anti-Cx43 antibody). A “CDR” is a region of hypervariability interspersed within regions that are more conserved, termed “framework regions” (FR).

Systematic identification of residues included in the CDRs have been developed by Kabat et al. (1991, Sequences of Proteins of Immunological Interest, 5th Ed., United States Public Health Service, National Institutes of Health, Bethesda). In some aspects, variable light chain (VL) CDRs are herein defined to include residues at positions 27-32 (CDR1), 50-56 (CDR2), and 91-97 (CDR3) of SEQ ID NO: 60. In some aspects, variable heavy chain (VH) CDRs are herein defined to include residues at positions 27-33 (CDR1), 52-56 (CDR2), and 95-102 (CDR3) of SEQ ID NO: 58.

The CDRs disclosed herein may also include variants. Generally, the amino acid identity between individual variant CDRs is at least 90%, 91%, 92%, 93%, 94%, 95%, 96%, 97%, 98%, 99%, or 100%. Thus, a “variant CDR” is one with the specified identity to the parent (e.g., SEQ ID NOs: 19, 20, 21, 31, 32 or 33) or reference CDR of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR (e.g., SEQ ID NOs: 19, 20, 21, 31, 32 or 33). For example, a “variant CDR” can be a sequence that contains 1, 2, 3 or 4 amino acid changes as compared to the parent or reference CDR of the invention, and shares or improves biological function, specificity and/or activity of the parent CDR.

In some aspects, any of CDR sequences disclosed herein can include a single amino acid change as compared to the parent or reference CDR. In some aspects, any of the CDR sequences disclosed herein can include at least two amino acid changes as compared to the parent or reference CDR. In some aspects, the amino acid change can be a change from a cysteine residue to another amino acid. In some aspects, the amino acid change can be a change from a glycine residue to another amino acid. The amino acid identity between individual variant CDRs can be at least 60%, 65%, 70%, 75%, 80%, 85%, 90%, 91%, 92%, 93%, 94%, 95%, 96%, 97%, 98%, 99% or 100%. Thus, a “variant CDR” can be one with the specified identity to the parent CDR of the invention, and shares biological function, including, but not limited to, at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR. For example, the parent CDR sequence can be one or more of SEQ ID NOs: 19, 20, 21, 31, 32, and/or 33. The variant CDR sequence can be at least 60%, 65%, 70%, 75%, 80%, 85%, 90%, 91%, 92%, 93%, 94%, 95%, 96%, 97%, 98%, 99% or 100% identical to any one of SEQ ID NOs: 19, 20, 21, 31, 32, and/or 33. The variant CDR sequence can also share at least 80%, 81%, 82%, 83%, 84%, 85%, 86%, 87%, 88%, 89%, 90%, 91%, 92%, 93%, 94%, 95%, 96%, 97%, 98%, or 99% of the specificity and/or activity of the parent CDR.

As discussed herein, minor variations in the amino acid sequences of any of the antibodies disclosed herein are contemplated as being encompassed by the instant disclosure, providing that the variations in the amino acid sequence maintains at least 75%, more preferably at least 80%, 90%, 95%, and most preferably 99% sequence identity to the parent sequence. In some aspects, conservative amino acid replacements are contemplated. Conservative replacements are those that take place within a family of amino acids that are related in their side chains. Genetically encoded amino acids are generally divided into families: (1) acidic=aspartate, glutamate; (2) basic=lysine, arginine, histidine; (3) nonpolar=alanine, valine, leucine, isoleucine, proline, phenylalanine, methionine, tryptophan; and (4) uncharged polar=glycine, asparagine, glutamine, cysteine, serine, threonine, tyrosine. More preferred families are: serine and threonine are aliphatic-hydroxy family; asparagine and glutamine are an amide-containing family; alanine, valine, leucine and isoleucine are an aliphatic family; and phenylalanine, tryptophan, and tyrosine are an aromatic family. For example, it is reasonable to expect that an isolated replacement of a leucine with an isoleucine or valine, an aspartate with a glutamate, a threonine with a serine, or a similar replacement of an amino acid with a structurally related amino acid will not have a major effect on the binding or properties of the resulting molecule, especially if the replacement does not involve an amino acid within a framework site. Whether an amino acid change results in a functional peptide can readily be determined by assaying the specific activity of the polypeptide derivative. Assays are known to one of ordinary skill in the art.

In some aspects, amino acid substitutions can be those which: (1) reduce susceptibility to proteolysis, (2) reduce susceptibility to oxidation, (3) alter binding affinity for forming protein complexes, (4) alter binding affinities, and (4) confer or modify other physiocochemical or functional properties of such analogs. In some aspects, single or multiple amino acid substitutions (preferably conservative amino acid substitutions) may be made in the non-CDR sequence of the heavy chain, the light chain or both. In some aspects, one or more amino acid substitutions can be made in one or more of the CDR sequences of the heavy chain, the light chain or both.

Many methods have been developed for chemical labeling and enhancement of the properties of antibodies and their common fragments, including the Fab and F(ab′)2 fragments. Somewhat selective reduction of some antibody disulfide bonds has been previously achieved, yielding antibodies and antibody fragments that can be labeled at defined sites, enhancing their utility and properties. Selective reduction of the two hinge disulfide bonds present in F(ab′)2 fragments using mild reduction has been useful. In some aspects, cysteine and methionine can be susceptible to rapid oxidation, which can negatively influence the cleavage of protecting groups during synthesis and the subsequent peptide purification. In some instances, cysteine residues in peptides used for antibody production can affect the avidity of the antibody, because free cysteines are uncommon in vivo and therefore may not be recognized by the native peptide structure. In some aspects, the disclosed antibodies and fragments thereof comprise a sequence where a cysteine reside outside of the CDR (e.g. in the non-CDR sequence of the heavy chain, the light chain or both) is substituted. In some aspects, cysteine can be replaced with serine and methionine replaced with norleucine (Nle). Multiple cysteine residues on a peptide or in one of the disclosed antibodies or fragments thereof may be susceptible to forming disulfide linkages unless a reducing agent such as dithiothreitol (DTT) is added to the buffer or the cysteine residues can be replaced with serine residues.

While the site or region for introducing an amino acid sequence variation is predetermined, the mutation per se need not be predetermined. For example, in order to optimize the performance of a mutation at a given site, random mutagenesis may be conducted at the target codon or region and the expressed antigen binding protein CDR variants screened for the optimal combination of desired activity. Techniques for making substitution mutations at predetermined sites in DNA having a known sequence are well known, for example, M13 primer mutagenesis and PCR mutagenesis. Screening of the mutants is done using assays of antigen binding protein activities as described herein.

Amino acid substitutions are typically of single residues; insertions usually will be on the order of from about one (1) to about twenty (20) amino acid residues, although considerably larger insertions may be tolerated. Deletions range from about one (1) to about twenty (20) amino acid residues, although in some cases deletions may be much larger.

Substitutions, deletions, insertions or any combination thereof may be used to arrive at a final derivative or variant. Generally these changes are done on a few amino acids to minimize the alteration of the molecule, particularly the immunogenicity and specificity of the antigen binding protein. However, larger changes may be tolerated in certain circumstances.

A “fragment antigen-binding fragment (Fab)” is a region of an antibody that binds to antigen. By “Fab” or “Fab region” as used herein is meant the polypeptide that comprises the VH, CH1, VL, and CL immunoglobulin domains. Fab may refer to this region in isolation, or this region in the context of a full length antibody, antibody fragment or Fab fusion protein, or any other antibody embodiments as outlined herein. Fab′ fragments differ from Fab fragments in that they include additional residues at the C-terminus of the Chi domain, including one or more cysteine residues from the antibody hinge region. The cysteine residues of the constant domains bear a free thiol group. F(ab′)2 antibody fragments are pairs of Fab′ fragments linked by cysteine residues in the hinge region. Other chemical couplings of antibody fragments are also known in the art.

By “Fv” or “Fv fragment” or “Fv region” as used herein is meant a polypeptide that comprises the VL and VH domains of a single antibody. The Fv region is a minimal fragment that contains a complete antigen-recognition and binding site consisting of one heavy chain and one light chain variable domain. The three CDRs of each variable domain interact to define an antigen-biding site on the surface of the VH-VL dimer. Collectively, the six CDRs confer antigen-binding specificity to the antibody. As would be known in the art, a “single-chain” antibody or “scFv” fragment is a single chain Fv variant formed when the VH and VL domains of an antibody are included in a single polypeptide chain that recognizes and binds an antigen. Typically, single-chain antibodies include a polypeptide linker between the VH and VL domains that allows the scFv to form a desired three-dimensional structure for antigen binding (see, e.g., Pluckthun, In The Pharmacology of Monoclonal Antibodies, Rosenburg and Moore Eds., Springer-Verlag, New York, 113:269-315. 1994).

By “framework” or “framework region” as used herein is meant the region of an antibody variable domain exclusive of those regions defined as CDRs. Each antibody variable domain framework can be further subdivided into the contiguous regions separated by the CDRs (FR1, FR2, FR3 and FR4).

The term “antigen-binding portion” of an antibody (or simply “antibody portion”), as used herein, refers to one or more fragments of an antibody that retain the ability to specifically bind to an antigen (e.g., Cx43 hemichannel). It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the VL/VK, VH, CL and CH1 domains; (ii) a F(ab′)2 fragment, a bivalent fragment comprising two Fab fragments linked by a disulfide bridge at the hinge region; (iii) a Fab′ fragment, which can be an Fab with part of the hinge region (see, Fundamental Immunology (Paul ed., 3rd ed. 1993); (iv) a Fd fragment consisting of the VH and CH1 domains; (v) a Fv fragment consisting of the VL and VH domains of a single arm of an antibody; (vi) a dAb fragment (Ward et al., (1989) Nature 341:544-546), which consists of a VH domain; (vii) an isolated complementarity determining region (CDR); and (viii) a nanobody, a heavy chain variable region containing a single variable domain and two constant domains.

The term “specifically binds” or “immunospecifically binds” is not intended to indicate that an antibody binds exclusively to its intended target. Rather, an antibody “specifically binds” if its affinity for its intended target is about 5-fold greater when compared to its affinity for a non-target molecule. Suitably there is no significant cross-reaction or cross-binding with undesired substances. The affinity of the antibody will, for example, be at least about 5-fold, such as 10-fold, such as 25-fold, especially 50-fold, and particularly 100-fold or more, greater for a target molecule than its affinity for a non-target molecule. In some embodiments, specific binding between an antibody or other binding agent and an antigen means a binding affinity of at least 10⁶ M⁻¹. Antibodies may, for example, bind with affinities of at least about 10⁷ M⁻¹, such as between about 10⁸ M⁻¹ to about 10⁹ M⁻¹, about 10⁹ M⁻¹ to about 10¹⁰ M⁻¹, or about 10¹⁰ M⁻¹ to about 10¹¹ M⁻¹. Antibodies may, for example, bind with an EC50 of 50 nM or less, 10 nM or less, 1 nM or less, 100 pM or less, or more preferably 10 pM or less. In some aspects, the antibodies can bind with an EC50 of about 60 μg/ml, 59 μg/ml, 58 μg/ml, 57 μg/ml, 56 μg/ml, 55 μg/ml, 54 μg/ml, 53 μg/ml, 52 μg/ml, 51 μg/ml, 50 μg/ml or less. In some aspects, the antibodies can bind with an EC50 of about 50 μg/ml, 49 μg/ml, 48 μg/ml, 47 μg/ml, 46 μg/ml, 45 μg/ml, 44 μg/ml, 43 μg/ml, 42 μg/ml, 41 μg/ml, 40 μg/ml or less. In some aspects, the antibodies can bind with an EC50 of about 40 μg/ml, 39 μg/ml, 38 μg/ml, 37 μg/ml, 36 μg/ml, 35 μg/ml, 34 μg/ml, 33 μg/ml, 32 μg/ml, 31 μg/ml, 30 μg/ml or less.

In some aspects, the antibodies described herein can specifically bind to their intended target. In some aspects, the antibodies described herein have no off site binding. For example, the antibodies described herein bind only to their intended target at a particular target site and do not bind or are not distributed to the heart, liver or spinal cord.

The antibodies described herein can be variants including, without limitation, a fragment (e.g., an Fab fragment or an F(ab′)2 fragment of, e.g., a tetrameric antibody), a fragment of an scFv or diabody, or a variant of a tetrameric antibody, an scFv, a diabody, or fragments thereof that differ by virtue of the addition and/or substitution of one or more amino acid residues. The antibody moiety can be further engineered as, for example, a di-diabody.

As is well known in the art, certain types of antibody fragments can be generated by enzymatic treatment of a “full-length” antibody. Digestion with papain produces two identical Fab fragments, each with a single antigen-binding site, and a residual Fc fragment. The Fab fragment also contains the constant domain of the light chain and the Chi domain of the heavy chain. In contrast, digestion with pepsin yields the F(ab′)2 fragment that has two antigen-binding sites and is still capable of cross-linking antigen.

In some aspects, the antibody can be a diabody. Diabodies are small antibody fragments that have two antigen-binding sites. Each fragment contains a VH domain concatenated to a VL domain. However, since the linker between the domains is too short to allow pairing between them on the same chain, the linked Vh-Vl domains are forced to pair with complementary domains of another chain, creating two antigen-binding sites. Diabodies are described more fully, for example, in EP 404,097; WO 93/11161; and Hollinger et al., Proc. Natl. Acad. Sci. USA 90:6444-6448, 1993.

In some aspects, an anti-Cx43 antibody or a fragment thereof that binds to at least a portion of Cx43 protein and inhibits or blocks the opening of Cx43 hemichannels and is associated with reduced Mmp13 and collagen X levels, decreased degree of synovitis or reduced synovial inflammation and improved cartilage integrity or reduced cartilage degradation are contemplated. In some aspects, an antibody or a fragment thereof that binds to at least a portion of Cx43 protein and inhibits or blocks the opening of Cx43 hemichannels and reduces Mmp13 levels and collagen X levels, decrease the degree of synovitis or reduced synovial inflammation, and improves the integrity of cartilage or reduced cartilage degradation are contemplated. In some aspects, the anti-Cx43 antibody can be a monoclonal antibody, polyclonal antibody or a humanized antibody. Thus, by known means and as described herein, polyclonal or monoclonal antibodies, antibody fragments, and binding domains and CDRs (including engineered forms of any of the foregoing) may be created that are specific to Cx43 protein, one or more of its respective epitopes, or conjugates of any of the foregoing, whether such antigens or epitopes are isolated from natural sources or are synthetic derivatives or variants of the natural compounds. Examples of epitope sequences are described in Application No. PCT/US2017/019605 (WO 2017-147561), which is hereby incorporated by reference for its teaching of epitope sequences that the anti-Cx43 antibodies and fragments thereof disclosed herein can bind.

Examples of anti-Cx43 antibody fragments suitable include without limitation: (i) the Fab fragment, consisting of VL, VH, CL, and CH1 domains; (ii) the “Fd” fragment consisting of the VII and Cm domains; (iii) the “Fv” fragment consisting of the VL and VH domains of a single antibody; (iv) the “dAb” fragment, which consists of a VH domain; (v) isolated CDR regions; (vi) F(ab′)2 fragments, a bivalent fragment comprising two linked Fab fragments; (vii) single chain Fv molecules (“scFv”), wherein a VII domain and a VL domain are linked by a peptide linker that allows the two domains to associate to form a binding domain; (viii) bi-specific single chain Fv dimers (see U.S. Pat. No. 5,091,513); and (ix) diabodies, multivalent or multispecific fragments constructed by gene fusion (US Patent App. Pub. 20050214860). Fv, scFv, or diabody molecules may be stabilized by the incorporation of disulphide bridges linking the VH and VL domains. Minibodies comprising a scFv joined to a CH3 domain may also be made (Hu et al., 1996).

Antibody-like binding peptidomimetics are also contemplated. Liu et al. (2003) describe “antibody like binding peptidomimetics” (ABiPs), which are peptides that act as pared-down antibodies and have certain advantages of longer serum half-life as well as less cumbersome synthesis methods.

Animals may be inoculated with an antigen, such as a Cx43 extracellular domain protein, in order to produce antibodies specific for Cx43 protein. Frequently an antigen is bound or conjugated to another molecule to enhance the immune response. As used herein, a conjugate is any peptide, polypeptide, protein, or non-proteinaceous substance bound to an antigen that is used to elicit an immune response in an animal. Antibodies produced in an animal in response to antigen inoculation comprise a variety of non-identical molecules (polyclonal antibodies) made from a variety of individual antibody producing B lymphocytes. A polyclonal antibody is a mixed population of antibody species, each of which may recognize a different epitope on the same antigen. Given the correct conditions for polyclonal antibody production in an animal, most of the antibodies in the animal's serum will recognize the collective epitopes on the antigenic compound to which the animal has been immunized. This specificity is further enhanced by affinity purification to select only those antibodies that recognize the antigen or epitope of interest.

As provided herein, a monoclonal antibody is a single species of antibody wherein every antibody molecule recognizes the same epitope because the antibody producing cells are derived from a single B-lymphocyte cell line. The methods for generating monoclonal antibodies (MAbs) generally begin along the same lines as those for preparing polyclonal antibodies. In some aspects, rodents such as mice and rats are used in generating monoclonal antibodies. In some aspects, rabbit, sheep, or frog cells are used in generating monoclonal antibodies. The use of rats is well known and may provide certain advantages. Mice (e.g., BALB/c mice) are routinely used and generally give a high percentage of stable fusions.

Hybridoma technology involves the fusion of a single B lymphocyte from a mouse previously immunized with a Cx43 antigen with an immortal myeloma cell (usually mouse myeloma). This technology provides a method to propagate a single antibody-producing cell for an indefinite number of generations, such that unlimited quantities of structurally identical antibodies having the same antigen or epitope specificity (monoclonal antibodies) may be produced.

Plasma B cells may be isolated from freshly prepared rabbit peripheral blood mononuclear cells of immunized rabbits and further selected for Cx43 binding cells. After enrichment of antibody producing B cells, total RNA may be isolated and cDNA synthesized. DNA sequences of antibody variable regions from both heavy chains and light chains may be amplified, constructed into a phage display Fab expression vector, and transformed into E. coli. A Cx43 specific binding Fab may be selected out through multiple rounds enrichment panning and sequenced. Selected Cx43 binding hits may be expressed as full length IgG in rabbit and rabbit/human chimeric forms using a mammalian expression vector system in human embryonic kidney (HEK293) cells (Invitrogen) and purified using a protein G resin with a fast protein liquid chromatography (FPLC) separation unit.

In some aspects, the anti-Cx43 antibody can be a chimeric antibody, for example, an antibody comprising antigen binding sequences from a non-human donor grafted to a heterologous non-human, human, or humanized sequence (e.g., framework and/or constant domain sequences). Methods have been developed to replace light and heavy chain constant domains of the monoclonal antibody with analogous domains of human origin, leaving the variable regions of the foreign antibody intact. Alternatively, “fully human” monoclonal antibodies can be produced in mice transgenic for human immunoglobulin genes. Methods have also been developed to convert variable domains of monoclonal antibodies to more human form by recombinantly constructing antibody variable domains having both rodent, for example, mouse, and human amino acid sequences. In “humanized” monoclonal antibodies, only the hypervariable CDR is derived from mouse monoclonal antibodies, and the framework and constant regions are derived from human amino acid sequences (see U.S. Pat. Nos. 5,091,513 and 6,881,557). It is thought that replacing amino acid sequences in the antibody that are characteristic of rodents with amino acid sequences found in the corresponding position of human antibodies will reduce the likelihood of adverse immune reaction during therapeutic use. A hybridoma or other cell producing an antibody may also be subject to genetic mutation or other changes, which may or may not alter the binding specificity of antibodies produced by the hybridoma.

Methods for producing polyclonal antibodies in various animal species, as well as for producing monoclonal antibodies of various types, including humanized, chimeric, and fully human, are well known in the art and highly predictable. For example, the following U.S. patents and patent applications provide enabling descriptions of such methods: U.S. Patent Application Nos. 2004/0126828 and 2002/0172677; and U.S. Pat. Nos. 3,817,837; 3,850,752; 3,939,350; 3,996,345; 4,196,265; 4,275,149; 4,277,437; 4,366,241; 4,469,797; 4,472,509; 4,606,855; 4,703,003; 4,742,159; 4,767,720; 4,816,567; 4,867,973; 4,938,948; 4,946,778; 5,021,236; 5,164,296; 5,196,066; 5,223,409; 5,403,484; 5,420,253; 5,565,332; 5,571,698; 5,627,052; 5,656,434; 5,770,376; 5,789,208; 5,821,337; 5,844,091; 5,858,657; 5,861,155; 5,871,907; 5,969,108; 6,054,297; 6,165,464; 6,365,157; 6,406,867; 6,709,659; 6,709,873; 6,753,407; 6,814,965; 6,849,259; 6,861,572; 6,875,434; and 6,891,024. All patents, patent application publications, and other publications cited herein and therein are hereby incorporated by reference in the present application.

Antibodies may be produced from any animal source, including birds and mammals. Preferably, the antibodies are ovine, murine (e.g., mouse and rat), rabbit, goat, guinea pig, camel, horse, or chicken. In addition, newer technology permits the development of and screening for human antibodies from human combinatorial antibody libraries. For example, bacteriophage antibody expression technology allows specific antibodies to be produced in the absence of animal immunization, as described in U.S. Pat. No. 6,946,546, which is incorporated herein by reference. These techniques are further described in: Marks (1992); Stemmer (1994); Gram et al. (1992); Barbas et al. (1994); and Schier et al. (1996).

It is fully expected that anti-Cx43 antibodies will have the ability to neutralize or counteract the effects of Cx43 regardless of the animal species, monoclonal cell line, or other source of the antibody. Certain animal species may be less preferable for generating therapeutic antibodies because they may be more likely to cause allergic response due to activation of the complement system through the “Fc” portion of the antibody. However, whole antibodies may be enzymatically digested into “Fc” (complement binding) fragment, and into antibody fragments having the binding domain or CDR. Removal of the Fc portion reduces the likelihood that the antigen antibody fragment will elicit an undesirable immunological response, and thus, antibodies without Fc may be preferential for prophylactic or therapeutic treatments. As described herein, antibodies may also be constructed so as to be chimeric or partially or fully human, so as to reduce or eliminate the adverse immunological consequences resulting from administering to an animal an antibody that has been produced in, or has sequences from, other species.

Proteins may be recombinant, or synthesized in vitro. Alternatively, a non-recombinant or recombinant protein may be isolated from bacteria. It is also contemplated that a bacteria containing such a variant may be implemented in compositions and methods. Consequently, a protein need not be isolated.

It is contemplated that in compositions there is between about 0.001 mg and about 10 mg of total polypeptide, peptide, and/or protein per ml. Thus, the concentration of protein in a composition can be about, at least about or at most about 0.001, 0.010, 0.050, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml or more (or any range derivable therein). Of this, about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% may be an antibody that binds Cx43.

An anti-Cx43 antibody or preferably an immunological portion of an anti-Cx43 antibody, can be chemically conjugated to, or expressed as, a fusion protein with other proteins. For purposes of this specification and the accompanying claims, all such fused proteins are included in the definition of antibodies or an immunological portion of an antibody.

Described herein are anti-Cx43 antibodies and anti-Cx43 antibody-like molecules against Cx43, polypeptides and peptides that are linked to at least one agent to form an antibody conjugate or payload. In order to increase the efficacy of antibody molecules as diagnostic or therapeutic agents, to the antibody can be linked or covalently bound or complexed to at least one desired molecule or moiety. Such a molecule or moiety may be, but is not limited to, at least one effector or reporter molecule. Effector molecules comprise molecules having a desired activity, e.g., cytotoxic activity. Non-limiting examples of effector molecules that have been attached to antibodies include toxins, therapeutic enzymes, antibiotics, radio-labeled nucleotides and the like. By contrast, a reporter molecule is defined as any moiety that may be detected using an assay. Non-limiting examples of reporter molecules that have been conjugated to antibodies include enzymes, radiolabels, haptens, fluorescent labels, phosphorescent molecules, chemiluminescent molecules, chromophores, luminescent molecules, photoaffinity molecules, colored particles or ligands, such as biotin.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody. Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate.

In some aspects, the anti-Cx43 antibodies described herein can comprise a heavy chain immunoglobulin variable region comprising complementarity determining region 1 (CDR1) comprising the sequence of SEQ ID NO: 19; CDR2 comprising the sequence of SEQ ID NO: 20; and a CDR3 comprising the sequence of SEQ ID NO: 21. Table 2 shows examples of CDRs of heavy chains of anti-Cx43 antibodies. Additional CDR sequences of heavy chains can be one or more of the heavy chain CDR sequences disclosed in Application No. PCT/US2017/019605 (WO 2017-147561), which is hereby incorporated by references for its teaching of heavy chain CDR sequences.

In some aspects, the anti-Cx43 antibodies described herein can comprise a light chain immunoglobulin variable region comprising complementarity determining region 1 (CDR1) comprising the sequence of SEQ ID NO: 31; CDR2 comprising the sequence of SEQ ID NO: 32; and a CDR3 comprising the sequence of SEQ ID NO: 33. Table 2 shows examples of CDRs in light chains of anti-Cx43 antibodies. Additional CDR sequences of light chains can be one or more the light chain CDR sequences disclosed in Application No. PCT/US2017/019605 (WO 2017-147561), which is hereby incorporated by references for its teaching of light chain CDR sequences.

In some aspects, the anti-Cx43 antibodies described herein can comprise a heavy chain immunoglobulin variable region comprising complementarity determining region 1 (CDR1) comprising the sequence of SEQ ID NO: 19; CDR2 comprising the sequence of SEQ ID NO: 20; and a CDR3 comprising the sequence of SEQ ID NO: 21; and a light chain immunoglobulin variable region comprising complementarity determining region 1 (CDR1) comprising the sequence of SEQ ID NO: 31; CDR2 comprising the sequence of SEQ ID NO: 32; and a CDR3 comprising the sequence of SEQ ID NO: 33.

In some aspects, the anti-Cx43 antibodies described herein can comprise a variable heavy chain comprising a sequence having at least 90% identity to a sequence set forth in SEQ ID NOs: 19, 20, or 21 (see, Table 2). In some aspects, the anti-Cx43 antibodies described herein comprises a variable heavy chain comprising a sequence having at least 90, 91, 92, 93, 94, 95, 96, 97, 98, 99, or 100% identity to a sequence set forth in SEQ ID NOs: 19, 20 or 21.

In some aspects, the anti-Cx43 antibodies described herein can comprise a variable light chain comprising a sequence having at least 90% identity to a sequence set forth in SEQ ID NOs: 31, 32 or 33 (see, Table 2). In some aspects, the anti-Cx43 antibodies described herein comprises a variable light chain comprising a sequence having at least 90, 91, 92, 93, 94, 95, 96, 97, 98, 99, or 100% identity to a sequence set forth in SEQ ID NOs: 31, 32 or 33.

Disclosed herein are nucleic acid sequences that encode M1H comprising the sequence of SEQ ID NO: 52. Disclosed herein are nucleic acid sequences that encode M1K1 comprising the sequence of SEQ ID NO: 54.

Disclosed herein are nucleic acid sequences encoding M1H comprising a variable heavy chain comprising a sequence having at least 90% identity to a sequence set forth in SEQ ID NO: 52 (see, Table 3). In some aspects, M1H comprises a variable heavy chain comprising a sequence having at least 90, 91, 92, 93, 94, 95, 96, 97, 98, 99 or 100% identity to a sequence set forth in SEQ ID NO: 52.

Disclosed herein are nucleic acid sequences encoding M1K1 comprising a variable light chain comprising a sequence having at least 90% identity to a sequence set forth in SEQ ID NO: 54 (see, Table 3). In some aspects, M1K1 comprises a variable light chain comprising a sequence having at least 90, 91, 92, 93, 94, 95, 96, 97, 98, 99 or 100% identity to a sequence set forth in SEQ ID NO: 54.

Disclosed herein are nucleic acid sequences that encode the M1H region comprising a heavy chain immunoglobulin variable region comprising a CDR1 comprising the sequence of SEQ ID NO: 16; a CDR2 comprising a comprising the sequence of SEQ ID NO: 17; a CDR3 comprising a comprising the sequence of SEQ ID NO: 18.

Disclosed herein are nucleic acid sequences that encode the M1K1 region comprising a light chain immunoglobulin variable region comprising a CDR1 comprising the sequence of SEQ ID NO: 28; a CDR2 comprising a comprising the sequence of SEQ ID NO: 29; a CDR3 comprising a comprising the sequence of SEQ ID NO: 30.

Disclosed herein are nucleic acid sequences that encode anti-Cx43 antibodies comprising a heavy chain immunoglobulin variable region comprising a CDR1 comprising the sequence of SEQ ID NO: 16; a CDR2 comprising a comprising the sequence of SEQ ID NO: 17; a CDR3 comprising a comprising the sequence of SEQ ID NO: 18; and a light chain immunoglobulin variable region comprising a CDR1 comprising the sequence of SEQ ID NO: 28; a CDR2 comprising a comprising the sequence of SEQ ID NO: 29; a CDR3 comprising a comprising the sequence of SEQ ID NO: 30.

Disclosed herein are anti-Cx43 antibodies or fragments thereof that bind to human Cx43. In some aspects, the anti-Cx43 antibodies or fragments thereof comprises a variable heavy chain comprising a sequence having at least 90% identity to one of the variable heavy chain amino acid sequences provided in Tables 2 or 4. In some aspects, the anti-Cx43 antibodies or fragments thereof comprises a variable heavy chain comprising a sequence having at least 90% identity to a sequence set forth in SEQ ID NO: 58. In some aspects, the anti-Cx43 antibodies or fragments thereof comprises a variable heavy chain comprising a sequence set forth in SEQ ID NO: 58.

Disclosed herein are anti-Cx43 antibodies or fragments thereof that bind to human Cx43. In some aspects, the anti-Cx43 antibodies or fragments thereof comprises a variable light chain comprising a sequence having at least 90% identity to one of the variable light chain amino acid sequences provided in Tables 2 or 4. In some aspects, the anti-Cx43 antibodies or fragments thereof comprises a variable light chain comprising a sequence having at least 90% identity to a sequence set forth in SEQ ID NO: 60. In some aspects, the anti-Cx43 antibodies or fragments thereof comprises a variable light chain comprising a sequence set forth in SEQ ID NOs: 60.

Disclosed herein are anti-Cx43 antibodies or fragments thereof that bind to human Cx-43 hemichannels. In some aspects, the anti-Cx43 antibodies or fragments thereof comprises a variable heavy chain comprising a sequence having at least 90% identity to a sequence set forth in SEQ ID NO: 58, and a variable light chain comprising a sequence having at least 90% identity to a sequence set forth in SEQ ID NO: 60. In some aspects, the anti-Cx43 antibody comprises a variable heavy chain comprising a sequence set forth in SEQ ID NO: 58 and a variable light chain comprising a sequence set forth in SEQ ID NO: 60.

In some aspects, the anti-Cx43 antibodies or fragments thereof comprises a M1H region. In some aspects, the M1H region comprises a heavy chain immunoglobulin variable region comprising a CDR1 comprising the sequence of SEQ ID NO: 19; a CDR2 comprising the sequence of SEQ ID NO: 20; and a CDR3 comprising the sequence of SEQ ID NO: 21.

In some aspects, the anti-Cx43 antibodies or fragments thereof comprises a M1K1 region. In some aspects, the M1K1 region comprises a light chain immunoglobulin variable region comprising a CDR1 comprising the sequence of SEQ ID NO: 31; a CDR2 comprising the sequence of 32; and a CDR3 comprising the sequence of SEQ ID NO: 33.

In some instances, the disclosed anti-Cx43 antibodies or fragments thereof further comprise a tag sequence.

Disclosed herein are nucleic acid sequences that encode the disclosed anti-Cx43 antibodies or fragments thereof. For example, disclosed are nucleic acid sequences comprising a variable heavy chain comprising a sequence having at least 90% identity to a sequence set forth in SEQ ID NO: 52. Disclosed herein are nucleic acid sequences that encode the disclosed anti-Cx43 antibodies or fragments thereof. For example, disclosed herein are nucleic acid sequences comprising a variable heavy chain comprising a sequence set forth in SEQ ID NO: 52. Also disclosed herein are nucleic acid sequences comprising a variable light chain comprising a sequence having at least 90% identity to a sequence set forth in SEQ ID NO: 54. Also disclosed are nucleic acid sequences comprising a variable light chain comprising a sequence set forth in SEQ ID NO: 54.

Disclosed herein are nucleic acid sequences comprising a variable heavy chain comprising a sequence having at least 90% identity to a sequence set forth in SEQ ID NO: 52; and a variable light chain comprising a sequence having at least 90% identity a sequence set forth in SEQ ID NO: 54. Disclosed are nucleic acid sequences comprising a variable heavy chain comprising a sequence set forth in SEQ ID NO: 52; and a variable light chain comprising a sequence set forth in SEQ ID NO: 54.

Disclosed herein are nucleic acid sequences capable of encoding a single chain variable fragment comprising a variable heavy chain comprising a sequence having at least 90% identity a sequence set forth in SEQ ID NO: 52.

Disclosed are nucleic acid sequences capable of encoding a single chain variable fragment comprising a variable light chain comprising a sequence having at least 90% identity a sequence set forth in SEQ ID NO: 54.

Disclosed are nucleic acid sequences capable of encoding a single chain variable fragment comprising a variable heavy chain comprising a sequence having at least 90% identity a sequence set forth in SEQ ID NO:58; and a variable light chain comprising a sequence having at least 90% identity a sequence set forth in SEQ ID NO:60.

In some instances, the disclosed anti-Cx43 antibodies or fragments thereof can be bispecific. For example, the antibody or fragment thereof can comprise a first Fab region comprising the heavy and light chain of SEQ ID NO: 58 and a second Fab region comprising the heavy and light chain of SEQ ID NO: 60, wherein the first and second Fab regions can be different.

In some instances, the bispecific anti-Cx43 antibodies can be trifunctional.

In some instances, the disclosed anti-Cx43 antibodies or fragments thereof can be mouse, human, humanized, chimeric, or a combination thereof.

In some instances, the disclosed anti-Cx43 antibodies or fragments thereof are monoclonal.

In further embodiments, the anti-Cx43 antibodies for use according to the embodiments can be any of those described in international (PCT) patent publication nos. WO 2015-027120 or WO 2017-147561, which are incorporated herein by reference for their teaching of antibodies, vectors and cells for making or expressing antibodies.

In some aspects, a first heavy chain region can comprise an amino acid sequence having an amino acid sequence of residues 13 to 37 of SEQ ID NO: 2; a second heavy chain region having an amino acid sequence corresponding to residues 46 to 66 of SEQ ID NO: 2; and a third heavy chain region comprising an amino acid sequence having an amino acid sequence of residues 97 to 116 of SEQ ID NO: 2.

In some aspects, the antibodies disclosed herein can include full length anti-Cx43 antibodies, antibody fragments, single chain antibodies, bispecific antibodies, minibodies, domain antibodies, synthetic antibodies and antibody fusions, and fragments thereof.

Methods

Disclosed herein are methods of treating or preventing osteoarthritis in a subject. Disclosed herein are methods of treating or preventing rheumatoid arthritis in a subject. Disclosed herein are methods of treating or preventing a tendon disorder or a tendon injury in a subject. Disclosed herein are methods of treating or preventing subchondral bone sclerosis in a subject. Disclosed herein are methods of reducing or ameliorating one or more symptoms of osteoarthritis in a subject. Disclosed herein are methods of reducing or ameliorating one or more symptoms of rheumatoid arthritis in a subject. Disclosed herein are methods of reducing or ameliorating one or more symptoms of a tendon disorder or a tendon injury in a subject. Disclosed herein are methods of reducing or ameliorating one or more symptoms of subchondral bone sclerosis in a subject. Disclosed herein are methods of reducing MMP13 or collagen X levels in a subject. Disclosed herein are methods of reducing cartilage degradation in a subject. Disclosed herein are methods of reducing synovial inflammation in a subject.

Disclosed herein are methods of treating or preventing osteoarthritis in a subject. In some aspects, the methods can comprise administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58. In some aspects, the methods can comprise administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60. In some aspects, the methods can comprise administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60. In some aspects, the antibody or fragment thereof comprises a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or a variant thereof; and/or c) a complementarity determining region (CDR3) comprising the sequence of SEQ ID NO: 21 or a variant thereof. In some aspects, the antibody or fragment thereof comprises a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 a variant thereof. In some aspects, any one of the CDR1, CDR2 or CDR3 of the heavy chain immunoglobulin variable region or the CDR1, CDR2, or CDR3 of the light chain immunoglobulin variable region can comprise at least one amino acid substitution as compared to the parent CDR. In some aspects, the at least one amino acid substitution is a cysteine residue to another amino acid or a glycine to another amino acid. In some aspects, the antibody or fragment thereof an comprise a heavy chain immunoglobulin variable region comprising: a) a complementarity determining region 1 (CDR1) comprising a sequence having at least 60% identity to a sequence set forth in SEQ ID NO: 19; b) a complementarity determining region 2 (CDR2) comprising a sequence having at least 60% identity to a sequence set forth in SEQ ID NO: 20; and/or c) a complementarity determining region 3 (CDR3) comprising a sequence having at least 60% identity to a sequence set forth in SEQ ID NO: 21. In some aspects, the antibody or fragment thereof can comprise a light chain immunoglobulin variable region comprising: a) a complementarity determining region 1 (CDR1) comprising a sequence having at least 60% identity to a sequence set forth in SEQ ID NO: 31; b) a complementarity determining region 2 (CDR2) comprising a sequence having at least 60% identity to a sequence set forth in SEQ ID NO: 32; and/or c) a complementarity determining region 3 (CDR3) comprising a sequence having at least 60% identity to a sequence set forth in SEQ ID NO: 33. In some aspects, the antibody or fragment thereof comprises: a) a heavy chain immunoglobulin variable region comprising: i) a first complementarity determining region 1 comprising a sequence having at least 60% identity to SEQ ID NO: 19; ii) a second complementarity determining region 2 comprising a sequence having at least 60% identity to SEQ ID NO: 20; and iii) a third complementarity determining region 3 comprising a sequence having at least 60% identity to SEQ ID NO: 21; and b) a light chain immunoglobulin variable region comprising: i) a first complementarity determining region 1 comprising a sequence having at least 60% identity to SEQ ID NO: 31; ii) a second complementarity determining region 1 comprising a sequence having at least 60% identity to SEQ ID NO: 32; and iii) a third complementarity determining region 1 comprising a sequence having at least 60% identity to SEQ ID NO: 33. In some aspects, the antibody or fragment thereof can comprise a heavy chain immunoglobulin variable region comprising: a) a complementarity determining region 1 (CDR1) comprising a sequence having a single amino acid change compared to a sequence set forth in SEQ ID NO: 19; b) a complementarity determining region 2 (CDR2) comprising a sequence having a single amino acid change compared to a sequence set forth in SEQ ID NO: 20; and/or c) a complementarity determining region 3 (CDR3) comprising a sequence having a single amino acid change compared to a sequence set forth in SEQ ID NO: 21. In some aspects, the antibody or fragment thereof can comprise region 1 (CDR1) comprising a sequence having a single amino acid change compared to a sequence set forth in SEQ ID NO: 31; b) a complementarity determining region 2 (CDR2) comprising a sequence having a single amino acid change compared to a sequence set forth in SEQ ID NO: 32; and/or c) a complementarity determining region 3 (CDR3) comprising a sequence having a single amino acid change compared to a sequence set forth in SEQ ID NO: 33. In some aspects, the antibody or fragment thereof can comprise: a) a heavy chain immunoglobulin variable region comprising: i) a first complementarity region 1 comprising a sequence having a single amino acid change compared to SEQ ID NO: 19; ii) a second complementarity region 2 comprising a sequence having a single amino acid change compared to SEQ ID NO: 20; and iii) a third complementarity region 3 comprising a sequence having a single amino acid change compared to SEQ ID NO: 21; and b) a light chain immunoglobulin variable region comprising: i) a first complementarity region 1 comprising a sequence having a single amino acid change compared to SEQ ID NO: 31; ii) a second complementarity region 1 comprising a sequence having a single amino acid change compared to SEQ ID NO: 32; and iii) a third complementarity region 1 comprising a sequence having a single amino acid change compared to SEQ ID NO: 33.

Disclosed herein are methods of treating or preventing rheumatoid arthritis in a subject. In some aspects, the methods can comprise administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58. In some aspects, the methods can comprise administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60. In some aspects, the methods can comprise administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60. In some aspects, the antibody or fragment thereof comprises a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or a variant thereof; and/or c) a complementarity determining region (CDR3) comprising the sequence of SEQ ID NO: 21 or a variant thereof. In some aspects, the antibody or fragment thereof comprises a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 a variant thereof. In some aspects, any one of the CDR1, CDR2 or CDR3 of the heavy chain immunoglobulin variable region or the CDR1, CDR2, or CDR3 of the light chain immunoglobulin variable region can comprise at least one amino acid substitution as compared to the parent CDR. In some aspects, the at least one amino acid substitution is a cysteine residue to another amino acid or a glycine to another amino acid. In some aspects, the antibody or fragment thereof an comprise a heavy chain immunoglobulin variable region comprising: a) a complementarity determining region 1 (CDR1) comprising a sequence having at least 60% identity to a sequence set forth in SEQ ID NO: 19; b) a complementarity determining region 2 (CDR2) comprising a sequence having at least 60% identity to a sequence set forth in SEQ ID NO: 20; and/or c) a complementarity determining region 3 (CDR3) comprising a sequence having at least 60% identity to a sequence set forth in SEQ ID NO: 21. In some aspects, the antibody or fragment thereof can comprise a light chain immunoglobulin variable region comprising: a) a complementarity determining region 1 (CDR1) comprising a sequence having at least 60% identity to a sequence set forth in SEQ ID NO: 31; b) a complementarity determining region 2 (CDR2) comprising a sequence having at least 60% identity to a sequence set forth in SEQ ID NO: 32; and/or c) a complementarity determining region 3 (CDR3) comprising a sequence having at least 60% identity to a sequence set forth in SEQ ID NO: 33. In some aspects, the antibody or fragment thereof comprises: a) a heavy chain immunoglobulin variable region comprising: i) a first complementarity determining region 1 comprising a sequence having at least 60% identity to SEQ ID NO: 19; ii) a second complementarity determining region 2 comprising a sequence having at least 60% identity to SEQ ID NO: 20; and iii) a third complementarity determining region 3 comprising a sequence having at least 60% identity to SEQ ID NO: 21; and b) a light chain immunoglobulin variable region comprising: i) a first complementarity determining region 1 comprising a sequence having at least 60% identity to SEQ ID NO: 31; ii) a second complementarity determining region 1 comprising a sequence having at least 60% identity to SEQ ID NO: 32; and iii) a third complementarity determining region 1 comprising a sequence having at least 60% identity to SEQ ID NO: 33. In some aspects, the antibody or fragment thereof can comprise a heavy chain immunoglobulin variable region comprising: a) a complementarity determining region 1 (CDR1) comprising a sequence having a single amino acid change compared to a sequence set forth in SEQ ID NO: 19; b) a complementarity determining region 2 (CDR2) comprising a sequence having a single amino acid change compared to a sequence set forth in SEQ ID NO: 20; and/or c) a complementarity determining region 3 (CDR3) comprising a sequence having a single amino acid change compared to a sequence set forth in SEQ ID NO: 21. In some aspects, the antibody or fragment thereof can comprise a light chain immunoglobulin variable region comprising: a) a complementarity determining region 1 (CDR1) comprising a sequence having a single amino acid change compared to a sequence set forth in SEQ ID NO: 31; b) a complementarity determining region 2 (CDR2) comprising a sequence having a single amino acid change compared to a sequence set forth in SEQ ID NO: 32; and/or c) a complementarity determining region 3 (CDR3) comprising a sequence having a single amino acid change compared to a sequence set forth in SEQ ID NO: 33. In some aspects, the antibody or fragment thereof can comprise: a) a heavy chain immunoglobulin variable region comprising: i) a first complementarity region 1 comprising a sequence having a single amino acid change compared to SEQ ID NO: 19; ii) a second complementarity region 2 comprising a sequence having a single amino acid change compared to SEQ ID NO: 20; and iii) a third complementarity region 3 comprising a sequence having a single amino acid change compared to SEQ ID NO: 21; and b) a light chain immunoglobulin variable region comprising: i) a first complementarity region 1 comprising a sequence having a single amino acid change compared to SEQ ID NO: 31; ii) a second complementarity region 1 comprising a sequence having a single amino acid change compared to SEQ ID NO: 32; and iii) a third complementarity region 1 comprising a sequence having a single amino acid change compared to SEQ ID NO: 33.

Disclosed herein are methods of treating or preventing a tendon disorder or a tendon injury in a subject. In some aspects, the methods can comprise administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58. In some aspects, the methods can comprise administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60. In some aspects, the methods can comprise administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60. In some aspects, the antibody or fragment thereof comprises a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or a variant thereof; and/or c) a complementarity determining region (CDR3) comprising the sequence of SEQ ID NO: 21 or a variant thereof. In some aspects, the antibody or fragment thereof comprises a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 a variant thereof. In some aspects, any one of the CDR1, CDR2 or CDR3 of the heavy chain immunoglobulin variable region or the CDR1, CDR2, or CDR3 of the light chain immunoglobulin variable region can comprise at least one amino acid substitution as compared to the parent CDR. In some aspects, the at least one amino acid substitution is a cysteine residue to another amino acid or a glycine to another amino acid. In some aspects, the antibody or fragment thereof an comprise a heavy chain immunoglobulin variable region comprising: a) a complementarity determining region 1 (CDR1) comprising a sequence having at least 60% identity to a sequence set forth in SEQ ID NO: 19; b) a complementarity determining region 2 (CDR2) comprising a sequence having at least 60% identity to a sequence set forth in SEQ ID NO: 20; and/or c) a complementarity determining region 3 (CDR3) comprising a sequence having at least 60% identity to a sequence set forth in SEQ ID NO: 21. In some aspects, the antibody or fragment thereof can comprise a light chain immunoglobulin variable region comprising: a) a complementarity determining region 1 (CDR1) comprising a sequence having at least 60% identity to a sequence set forth in SEQ ID NO: 31; b) a complementarity determining region 2 (CDR2) comprising a sequence having at least 60% identity to a sequence set forth in SEQ ID NO: 32; and/or c) a complementarity determining region 3 (CDR3) comprising a sequence having at least 60% identity to a sequence set forth in SEQ ID NO: 33. In some aspects, the antibody or fragment thereof comprises: a) a heavy chain immunoglobulin variable region comprising: i) a first complementarity determining region 1 comprising a sequence having at least 60% identity to SEQ ID NO: 19; ii) a second complementarity determining region 2 comprising a sequence having at least 60% identity to SEQ ID NO: 20; and iii) a third complementarity determining region 3 comprising a sequence having at least 60% identity to SEQ ID NO: 21; and b) a light chain immunoglobulin variable region comprising: i) a first complementarity determining region 1 comprising a sequence having at least 60% identity to SEQ ID NO: 31; ii) a second complementarity determining region 1 comprising a sequence having at least 60% identity to SEQ ID NO: 32; and iii) a third complementarity determining region 1 comprising a sequence having at least 60% identity to SEQ ID NO: 33. In some aspects, the antibody or fragment thereof can comprise a heavy chain immunoglobulin variable region comprising: a) a complementarity determining region 1 (CDR1) comprising a sequence having a single amino acid change compared to a sequence set forth in SEQ ID NO: 19; b) a complementarity determining region 2 (CDR2) comprising a sequence having a single amino acid change compared to a sequence set forth in SEQ ID NO: 20; and/or c) a complementarity determining region 3 (CDR3) comprising a sequence having a single amino acid change compared to a sequence set forth in SEQ ID NO: 21. In some aspects, the antibody or fragment thereof can comprise a light chain immunoglobulin variable region comprising: a) a complementarity determining region 1 (CDR1) comprising a sequence having a single amino acid change compared to a sequence set forth in SEQ ID NO: 31; b) a complementarity determining region 2 (CDR2) comprising a sequence having a single amino acid change compared to a sequence set forth in SEQ ID NO: 32; and/or c) a complementarity determining region 3 (CDR3) comprising a sequence having a single amino acid change compared to a sequence set forth in SEQ ID NO: 33. In some aspects, the antibody or fragment thereof can comprise: a) a heavy chain immunoglobulin variable region comprising: i) a first complementarity region 1 comprising a sequence having a single amino acid change compared to SEQ ID NO: 19; ii) a second complementarity region 2 comprising a sequence having a single amino acid change compared to SEQ ID NO: 20; and iii) a third complementarity region 3 comprising a sequence having a single amino acid change compared to SEQ ID NO: 21; and b) a light chain immunoglobulin variable region comprising: i) a first complementarity region 1 comprising a sequence having a single amino acid change compared to SEQ ID NO: 31; ii) a second complementarity region 1 comprising a sequence having a single amino acid change compared to SEQ ID NO: 32; and iii) a third complementarity region 1 comprising a sequence having a single amino acid change compared to SEQ ID NO: 33.

Disclosed herein are methods of reducing MMP13 or collagen X levels. In some aspects, the methods can comprise administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58. In some aspects, the methods can comprise administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60. In some aspects, the methods can comprise administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60. In some aspects, the antibody or fragment thereof comprises a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or a variant thereof; and/or c) a complementarity determining region (CDR3) comprising the sequence of SEQ ID NO: 21 or a variant thereof. In some aspects, the antibody or fragment thereof comprises a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 a variant thereof. In some aspects, any one of the CDR1, CDR2 or CDR3 of the heavy chain immunoglobulin variable region or the CDR1, CDR2, or CDR3 of the light chain immunoglobulin variable region can comprise at least one amino acid substitution as compared to the parent CDR. In some aspects, the at least one amino acid substitution is a cysteine residue to another amino acid or a glycine to another amino acid. In some aspects, the antibody or fragment thereof an comprise a heavy chain immunoglobulin variable region comprising: a) a complementarity determining region 1 (CDR1) comprising a sequence having at least 60% identity to a sequence set forth in SEQ ID NO: 19; b) a complementarity determining region 2 (CDR2) comprising a sequence having at least 60% identity to a sequence set forth in SEQ ID NO: 20; and/or c) a complementarity determining region 3 (CDR3) comprising a sequence having at least 60% identity to a sequence set forth in SEQ ID NO: 21. In some aspects, the antibody or fragment thereof can comprise a light chain immunoglobulin variable region comprising: a) a complementarity determining region 1 (CDR1) comprising a sequence having at least 60% identity to a sequence set forth in SEQ ID NO: 31; b) a complementarity determining region 2 (CDR2) comprising a sequence having at least 60% identity to a sequence set forth in SEQ ID NO: 32; and/or c) a complementarity determining region 3 (CDR3) comprising a sequence having at least 60% identity to a sequence set forth in SEQ ID NO: 33. In some aspects, the antibody or fragment thereof comprises: a) a heavy chain immunoglobulin variable region comprising: i) a first complementarity determining region 1 comprising a sequence having at least 60% identity to SEQ ID NO: 19; ii) a second complementarity determining region 2 comprising a sequence having at least 60% identity to SEQ ID NO: 20; and iii) a third complementarity determining region 3 comprising a sequence having at least 60% identity to SEQ ID NO: 21; and b) a light chain immunoglobulin variable region comprising: i) a first complementarity determining region 1 comprising a sequence having at least 60% identity to SEQ ID NO: 31; ii) a second complementarity determining region 1 comprising a sequence having at least 60% identity to SEQ ID NO: 32; and iii) a third complementarity determining region 1 comprising a sequence having at least 60% identity to SEQ ID NO: 33. In some aspects, the antibody or fragment thereof can comprise a heavy chain immunoglobulin variable region comprising: a) a complementarity determining region 1 (CDR1) comprising a sequence having a single amino acid change compared to a sequence set forth in SEQ ID NO: 19; b) a complementarity determining region 2 (CDR2) comprising a sequence having a single amino acid change compared to a sequence set forth in SEQ ID NO: 20; and/or c) a complementarity determining region 3 (CDR3) comprising a sequence having a single amino acid change compared to a sequence set forth in SEQ ID NO: 21. In some aspects, the antibody or fragment thereof can comprise a light chain immunoglobulin variable region comprising: a) a complementarity determining region 1 (CDR1) comprising a sequence having a single amino acid change compared to a sequence set forth in SEQ ID NO: 31; b) a complementarity determining region 2 (CDR2) comprising a sequence having a single amino acid change compared to a sequence set forth in SEQ ID NO: 32; and/or c) a complementarity determining region 3 (CDR3) comprising a sequence having a single amino acid change compared to a sequence set forth in SEQ ID NO: 33. In some aspects, the antibody or fragment thereof can comprise: a) a heavy chain immunoglobulin variable region comprising: i) a first complementarity region 1 comprising a sequence having a single amino acid change compared to SEQ ID NO: 19; ii) a second complementarity region 2 comprising a sequence having a single amino acid change compared to SEQ ID NO: 20; and iii) a third complementarity region 3 comprising a sequence having a single amino acid change compared to SEQ ID NO: 21; and b) a light chain immunoglobulin variable region comprising: i) a first complementarity region 1 comprising a sequence having a single amino acid change compared to SEQ ID NO: 31; ii) a second complementarity region 1 comprising a sequence having a single amino acid change compared to SEQ ID NO: 32; and iii) a third complementarity region 1 comprising a sequence having a single amino acid change compared to SEQ ID NO: 33.

Disclosed herein are methods of reducing cartilage degradation in a subject. In some aspects, the methods can comprise administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58. In some aspects, the methods can comprise administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60. In some aspects, the methods can comprise administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60. In some aspects, the antibody or fragment thereof comprises a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or a variant thereof; and/or c) a complementarity determining region (CDR3) comprising the sequence of SEQ ID NO: 21 or a variant thereof. In some aspects, the antibody or fragment thereof comprises a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 a variant thereof. In some aspects, any one of the CDR1, CDR2 or CDR3 of the heavy chain immunoglobulin variable region or the CDR1, CDR2, or CDR3 of the light chain immunoglobulin variable region can comprise at least one amino acid substitution as compared to the parent CDR. In some aspects, the at least one amino acid substitution is a cysteine residue to another amino acid or a glycine to another amino acid. In some aspects, the antibody or fragment thereof an comprise a heavy chain immunoglobulin variable region comprising: a) a complementarity determining region 1 (CDR1) comprising a sequence having at least 60% identity to a sequence set forth in SEQ ID NO: 19; b) a complementarity determining region 2 (CDR2) comprising a sequence having at least 60% identity to a sequence set forth in SEQ ID NO: 20; and/or c) a complementarity determining region 3 (CDR3) comprising a sequence having at least 60% identity to a sequence set forth in SEQ ID NO: 21. In some aspects, the antibody or fragment thereof can comprise a light chain immunoglobulin variable region comprising: a) a complementarity determining region 1 (CDR1) comprising a sequence having at least 60% identity to a sequence set forth in SEQ ID NO: 31; b) a complementarity determining region 2 (CDR2) comprising a sequence having at least 60% identity to a sequence set forth in SEQ ID NO: 32; and/or c) a complementarity determining region 3 (CDR3) comprising a sequence having at least 60% identity to a sequence set forth in SEQ ID NO: 33. In some aspects, the antibody or fragment thereof comprises: a) a heavy chain immunoglobulin variable region comprising: i) a first complementarity determining region 1 comprising a sequence having at least 60% identity to SEQ ID NO: 19; ii) a second complementarity determining region 2 comprising a sequence having at least 60% identity to SEQ ID NO: 20; and iii) a third complementarity determining region 3 comprising a sequence having at least 60% identity to SEQ ID NO: 21; and b) a light chain immunoglobulin variable region comprising: i) a first complementarity determining region 1 comprising a sequence having at least 60% identity to SEQ ID NO: 31; ii) a second complementarity determining region 1 comprising a sequence having at least 60% identity to SEQ ID NO: 32; and iii) a third complementarity determining region 1 comprising a sequence having at least 60% identity to SEQ ID NO: 33. In some aspects, the antibody or fragment thereof can comprise a heavy chain immunoglobulin variable region comprising: a) a complementarity determining region 1 (CDR1) comprising a sequence having a single amino acid change compared to a sequence set forth in SEQ ID NO: 19; b) a complementarity determining region 2 (CDR2) comprising a sequence having a single amino acid change compared to a sequence set forth in SEQ ID NO: 20; and/or c) a complementarity determining region 3 (CDR3) comprising a sequence having a single amino acid change compared to a sequence set forth in SEQ ID NO: 21. In some aspects, the antibody or fragment thereof can comprise region 1 (CDR1) comprising a sequence having a single amino acid change compared to a sequence set forth in SEQ ID NO: 31; b) a complementarity determining region 2 (CDR2) comprising a sequence having a single amino acid change compared to a sequence set forth in SEQ ID NO: 32; and/or c) a complementarity determining region 3 (CDR3) comprising a sequence having a single amino acid change compared to a sequence set forth in SEQ ID NO: 33. In some aspects, the antibody or fragment thereof can comprise: a) a heavy chain immunoglobulin variable region comprising: i) a first complementarity region 1 comprising a sequence having a single amino acid change compared to SEQ ID NO: 19; ii) a second complementarity region 2 comprising a sequence having a single amino acid change compared to SEQ ID NO: 20; and iii) a third complementarity region 3 comprising a sequence having a single amino acid change compared to SEQ ID NO: 21; and b) a light chain immunoglobulin variable region comprising: i) a first complementarity region 1 comprising a sequence having a single amino acid change compared to SEQ ID NO: 31; ii) a second complementarity region 1 comprising a sequence having a single amino acid change compared to SEQ ID NO: 32; and iii) a third complementarity region 1 comprising a sequence having a single amino acid change compared to SEQ ID NO: 33.

Disclosed herein are methods of reducing synovial inflammation in a subject. In some aspects, the methods can comprise administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58. In some aspects, the methods can comprise administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60. In some aspects, the methods can comprise administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60. In some aspects, the antibody or fragment thereof comprises a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or a variant thereof; and/or c) a complementarity determining region (CDR3) comprising the sequence of SEQ ID NO: 21 or a variant thereof. In some aspects, the antibody or fragment thereof comprises a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 a variant thereof. In some aspects, any one of the CDR1, CDR2 or CDR3 of the heavy chain immunoglobulin variable region or the CDR1, CDR2, or CDR3 of the light chain immunoglobulin variable region can comprise at least one amino acid substitution as compared to the parent CDR. In some aspects, the at least one amino acid substitution is a cysteine residue to another amino acid or a glycine to another amino acid. In some aspects, the antibody or fragment thereof an comprise a heavy chain immunoglobulin variable region comprising: a) a complementarity determining region 1 (CDR1) comprising a sequence having at least 60% identity to a sequence set forth in SEQ ID NO: 19; b) a complementarity determining region 2 (CDR2) comprising a sequence having at least 60% identity to a sequence set forth in SEQ ID NO: 20; and/or c) a complementarity determining region 3 (CDR3) comprising a sequence having at least 60% identity to a sequence set forth in SEQ ID NO: 21. In some aspects, the antibody or fragment thereof can comprise a light chain immunoglobulin variable region comprising: a) a complementarity determining region 1 (CDR1) comprising a sequence having at least 60% identity to a sequence set forth in SEQ ID NO: 31; b) a complementarity determining region 2 (CDR2) comprising a sequence having at least 60% identity to a sequence set forth in SEQ ID NO: 32; and/or c) a complementarity determining region 3 (CDR3) comprising a sequence having at least 60% identity to a sequence set forth in SEQ ID NO: 33. In some aspects, the antibody or fragment thereof comprises: a) a heavy chain immunoglobulin variable region comprising: i) a first complementarity determining region 1 comprising a sequence having at least 60% identity to SEQ ID NO: 19; ii) a second complementarity determining region 2 comprising a sequence having at least 60% identity to SEQ ID NO: 20; and iii) a third complementarity determining region 3 comprising a sequence having at least 60% identity to SEQ ID NO: 21; and b) a light chain immunoglobulin variable region comprising: i) a first complementarity determining region 1 comprising a sequence having at least 60% identity to SEQ ID NO: 31; ii) a second complementarity determining region 1 comprising a sequence having at least 60% identity to SEQ ID NO: 32; and iii) a third complementarity determining region 1 comprising a sequence having at least 60% identity to SEQ ID NO: 33. In some aspects, the antibody or fragment thereof can comprise a heavy chain immunoglobulin variable region comprising: a) a complementarity determining region 1 (CDR1) comprising a sequence having a single amino acid change compared to a sequence set forth in SEQ ID NO: 19; b) a complementarity determining region 2 (CDR2) comprising a sequence having a single amino acid change compared to a sequence set forth in SEQ ID NO: 20; and/or c) a complementarity determining region 3 (CDR3) comprising a sequence having a single amino acid change compared to a sequence set forth in SEQ ID NO: 21. In some aspects, the antibody or fragment thereof can comprise a light chain immunoglobulin variable region comprising: a) a complementarity determining region 1 (CDR1) comprising a sequence having a single amino acid change compared to a sequence set forth in SEQ ID NO: 31; b) a complementarity determining region 2 (CDR2) comprising a sequence having a single amino acid change compared to a sequence set forth in SEQ ID NO: 32; and/or c) a complementarity determining region 3 (CDR3) comprising a sequence having a single amino acid change compared to a sequence set forth in SEQ ID NO: 33. In some aspects, the antibody or fragment thereof can comprise: a) a heavy chain immunoglobulin variable region comprising: i) a first complementarity region 1 comprising a sequence having a single amino acid change compared to SEQ ID NO: 19; ii) a second complementarity region 2 comprising a sequence having a single amino acid change compared to SEQ ID NO: 20; and iii) a third complementarity region 3 comprising a sequence having a single amino acid change compared to SEQ ID NO: 21; and b) a light chain immunoglobulin variable region comprising: i) a first complementarity region 1 comprising a sequence having a single amino acid change compared to SEQ ID NO: 31; ii) a second complementarity region 1 comprising a sequence having a single amino acid change compared to SEQ ID NO: 32; and iii) a third complementarity region 1 comprising a sequence having a single amino acid change compared to SEQ ID NO: 33.

Disclosed herein are methods of treating or preventing subchondral bone sclerosis in a subject. In some aspects, the methods can comprise administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58. In some aspects, the methods can comprise administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60. In some aspects, the methods can comprise administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60. In some aspects, the antibody or fragment thereof comprises a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or a variant thereof; and/or c) a complementarity determining region (CDR3) comprising the sequence of SEQ ID NO: 21 or a variant thereof. In some aspects, the antibody or fragment thereof comprises a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 a variant thereof. In some aspects, any one of the CDR1, CDR2 or CDR3 of the heavy chain immunoglobulin variable region or the CDR1, CDR2, or CDR3 of the light chain immunoglobulin variable region can comprise at least one amino acid substitution as compared to the parent CDR. In some aspects, the at least one amino acid substitution is a cysteine residue to another amino acid or a glycine to another amino acid. In some aspects, the antibody or fragment thereof an comprise a heavy chain immunoglobulin variable region comprising: a) a complementarity determining region 1 (CDR1) comprising a sequence having at least 60% identity to a sequence set forth in SEQ ID NO: 19; b) a complementarity determining region 2 (CDR2) comprising a sequence having at least 60% identity to a sequence set forth in SEQ ID NO: 20; and/or c) a complementarity determining region 3 (CDR3) comprising a sequence having at least 60% identity to a sequence set forth in SEQ ID NO: 21. In some aspects, the antibody or fragment thereof can comprise a light chain immunoglobulin variable region comprising: a) a complementarity determining region 1 (CDR1) comprising a sequence having at least 60% identity to a sequence set forth in SEQ ID NO: 31; b) a complementarity determining region 2 (CDR2) comprising a sequence having at least 60% identity to a sequence set forth in SEQ ID NO: 32; and/or c) a complementarity determining region 3 (CDR3) comprising a sequence having at least 60% identity to a sequence set forth in SEQ ID NO: 33. In some aspects, the antibody or fragment thereof comprises: a) a heavy chain immunoglobulin variable region comprising: i) a first complementarity determining region 1 comprising a sequence having at least 60% identity to SEQ ID NO: 19; ii) a second complementarity determining region 2 comprising a sequence having at least 60% identity to SEQ ID NO: 20; and iii) a third complementarity determining region 3 comprising a sequence having at least 60% identity to SEQ ID NO: 21; and b) a light chain immunoglobulin variable region comprising: i) a first complementarity determining region 1 comprising a sequence having at least 60% identity to SEQ ID NO: 31; ii) a second complementarity determining region 1 comprising a sequence having at least 60% identity to SEQ ID NO: 32; and iii) a third complementarity determining region 1 comprising a sequence having at least 60% identity to SEQ ID NO: 33. In some aspects, the antibody or fragment thereof can comprise a heavy chain immunoglobulin variable region comprising: a) a complementarity determining region 1 (CDR1) comprising a sequence having a single amino acid change compared to a sequence set forth in SEQ ID NO: 19; b) a complementarity determining region 2 (CDR2) comprising a sequence having a single amino acid change compared to a sequence set forth in SEQ ID NO: 20; and/or c) a complementarity determining region 3 (CDR3) comprising a sequence having a single amino acid change compared to a sequence set forth in SEQ ID NO: 21. In some aspects, the antibody or fragment thereof can comprise a light chain immunoglobulin variable region comprising: a) a complementarity determining region 1 (CDR1) comprising a sequence having a single amino acid change compared to a sequence set forth in SEQ ID NO: 31; b) a complementarity determining region 2 (CDR2) comprising a sequence having a single amino acid change compared to a sequence set forth in SEQ ID NO: 32; and/or c) a complementarity determining region 3 (CDR3) comprising a sequence having a single amino acid change compared to a sequence set forth in SEQ ID NO: 33. In some aspects, the antibody or fragment thereof can comprise: a) a heavy chain immunoglobulin variable region comprising: i) a first complementarity region 1 comprising a sequence having a single amino acid change compared to SEQ ID NO: 19; ii) a second complementarity region 2 comprising a sequence having a single amino acid change compared to SEQ ID NO: 20; and iii) a third complementarity region 3 comprising a sequence having a single amino acid change compared to SEQ ID NO: 21; and b) a light chain immunoglobulin variable region comprising: i) a first complementarity region 1 comprising a sequence having a single amino acid change compared to SEQ ID NO: 31; ii) a second complementarity region 1 comprising a sequence having a single amino acid change compared to SEQ ID NO: 32; and iii) a third complementarity region 1 comprising a sequence having a single amino acid change compared to SEQ ID NO: 33.

Disclosed herein are methods of reducing or ameliorating one or more symptoms of osteoarthritis in a subject. In some aspects, the methods can comprise administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58. In some aspects, the methods can comprise administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60. In some aspects, the methods can comprise administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60. In some aspects, the antibody or fragment thereof comprises a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or a variant thereof; and/or c) a complementarity determining region (CDR3) comprising the sequence of SEQ ID NO: 21 or a variant thereof. In some aspects, the antibody or fragment thereof comprises a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 a variant thereof. In some aspects, any one of the CDR1, CDR2 or CDR3 of the heavy chain immunoglobulin variable region or the CDR1, CDR2, or CDR3 of the light chain immunoglobulin variable region can comprise at least one amino acid substitution as compared to the parent CDR. In some aspects, the at least one amino acid substitution is a cysteine residue to another amino acid or a glycine to another amino acid. In some aspects, the antibody or fragment thereof an comprise a heavy chain immunoglobulin variable region comprising: a) a complementarity determining region 1 (CDR1) comprising a sequence having at least 60% identity to a sequence set forth in SEQ ID NO: 19; b) a complementarity determining region 2 (CDR2) comprising a sequence having at least 60% identity to a sequence set forth in SEQ ID NO: 20; and/or c) a complementarity determining region 3 (CDR3) comprising a sequence having at least 60% identity to a sequence set forth in SEQ ID NO: 21. In some aspects, the antibody or fragment thereof can comprise a light chain immunoglobulin variable region comprising: a) a complementarity determining region 1 (CDR1) comprising a sequence having at least 60% identity to a sequence set forth in SEQ ID NO: 31; b) a complementarity determining region 2 (CDR2) comprising a sequence having at least 60% identity to a sequence set forth in SEQ ID NO: 32; and/or c) a complementarity determining region 3 (CDR3) comprising a sequence having at least 60% identity to a sequence set forth in SEQ ID NO: 33. In some aspects, the antibody or fragment thereof comprises: a) a heavy chain immunoglobulin variable region comprising: i) a first complementarity determining region 1 comprising a sequence having at least 60% identity to SEQ ID NO: 19; ii) a second complementarity determining region 2 comprising a sequence having at least 60% identity to SEQ ID NO: 20; and iii) a third complementarity determining region 3 comprising a sequence having at least 60% identity to SEQ ID NO: 21; and b) a light chain immunoglobulin variable region comprising: i) a first complementarity determining region 1 comprising a sequence having at least 60% identity to SEQ ID NO: 31; ii) a second complementarity determining region 1 comprising a sequence having at least 60% identity to SEQ ID NO: 32; and iii) a third complementarity determining region 1 comprising a sequence having at least 60% identity to SEQ ID NO: 33. In some aspects, the antibody or fragment thereof can comprise a heavy chain immunoglobulin variable region comprising: a) a complementarity determining region 1 (CDR1) comprising a sequence having a single amino acid change compared to a sequence set forth in SEQ ID NO: 19; b) a complementarity determining region 2 (CDR2) comprising a sequence having a single amino acid change compared to a sequence set forth in SEQ ID NO: 20; and/or c) a complementarity determining region 3 (CDR3) comprising a sequence having a single amino acid change compared to a sequence set forth in SEQ ID NO: 21. In some aspects, the antibody or fragment thereof can comprise a light chain immunoglobulin variable region comprising: a) a complementarity determining region 1 (CDR1) comprising a sequence having a single amino acid change compared to a sequence set forth in SEQ ID NO: 31; b) a complementarity determining region 2 (CDR2) comprising a sequence having a single amino acid change compared to a sequence set forth in SEQ ID NO: 32; and/or c) a complementarity determining region 3 (CDR3) comprising a sequence having a single amino acid change compared to a sequence set forth in SEQ ID NO: 33. In some aspects, the antibody or fragment thereof can comprise: a) a heavy chain immunoglobulin variable region comprising: i) a first complementarity region 1 comprising a sequence having a single amino acid change compared to SEQ ID NO: 19; ii) a second complementarity region 2 comprising a sequence having a single amino acid change compared to SEQ ID NO: 20; and iii) a third complementarity region 3 comprising a sequence having a single amino acid change compared to SEQ ID NO: 21; and b) a light chain immunoglobulin variable region comprising: i) a first complementarity region 1 comprising a sequence having a single amino acid change compared to SEQ ID NO: 31; ii) a second complementarity region 1 comprising a sequence having a single amino acid change compared to SEQ ID NO: 32; and iii) a third complementarity region 1 comprising a sequence having a single amino acid change compared to SEQ ID NO: 33.

In some aspects, the methods can comprise administering to the subject a therapeutically effective amount of an anti-connexin 43 antibody (i.e., anti-Cx43 antibody) or fragment thereof. In some aspects, the anti-Cx43 antibody or fragment thereof can comprise a variable heavy chain comprising a sequence having at least 90% identity to a sequence set forth in SEQ ID NO: 58. In some aspects, the anti-Cx43 antibody or fragment thereof can comprise a variable light chain comprising a sequence having at least 90% identity to a sequence set forth in SEQ ID NO: 60. In some aspects, the anti-Cx43 antibody or fragment thereof can comprise a variable heavy chain comprising a sequence having at least 90% identity to a sequence set forth in SEQ ID NO: 58 and a variable light chain comprising a sequence having at least 90% identity to a sequence set forth in SEQ ID NO: 60.

In some aspects, the methods can reduce MMP13 levels or collagen X levels. In some aspects, the methods can reduce cartilage degradation. In some aspects, the methods can reducing synovial inflammation. In some aspects, the methods can reduce or ameliorate one or more symptoms of osteoarthritis, rheumatoid arthritis, a tendon disorder or a tendon injury or subchondral bone sclerosis.

In some aspects, the anti-Cx43 antibody or fragment thereof can comprise: a heavy chain immunoglobulin variable region comprising: a first complementarity determining region 1 comprising a sequence having at least 60% identity to SEQ ID NO: 19; a second complementarity determining region 2 comprising a sequence having at least 60% identity to SEQ ID NO: 20; and a third complementarity determining region 3 comprising a sequence having at least 60% identity to SEQ ID NO: 21. In some aspects, the anti-Cx43 antibody or fragment thereof can comprise: a light chain immunoglobulin variable region comprising: a first complementarity determining region 1 comprising a sequence having at least 60% identity to SEQ ID NO: 31; a second complementarity determining region 2 comprising a sequence having at least 60% identity to SEQ ID NO: 32; and a third complementarity determining region 3 comprising a sequence having at least 60% identity to SEQ ID NO: 33.

In some aspects, the anti-Cx43 antibody or fragment thereof can comprise: a heavy chain immunoglobulin variable region comprising: a first complementarity determining region 1 comprising a sequence having a single amino acid change compared to SEQ ID NO: 19; a second complementarity determining region 2 comprising a sequence a single amino acid change compared to SEQ ID NO: 20; and a third complementarity determining region 3 comprising a sequence a single amino acid change compared to SEQ ID NO: 21. In some aspects, the anti-Cx43 antibody or fragment thereof can comprise: a light chain immunoglobulin variable determining region comprising: a first complementarity determining region 1 comprising a sequence having a single amino acid change compared to SEQ ID NO: 31; a second complementarity determining region 2 comprising a sequence having a single amino acid change compared to SEQ ID NO: 32; and a third complementarity determining region 3 comprising a sequence having a single amino acid change compared to SEQ ID NO: 33.

In some aspects, the anti-Cx43 antibody or fragment thereof can comprise a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or a variant thereof; and/or c) a complementarity determining region (CDR3) comprising the sequence of SEQ ID NO: 21 or a variant thereof. In some aspects, any one of the heavy chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid.

In some aspects the anti-Cx43 antibody or fragment thereof can comprise a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 or a variant thereof. In some aspects, any one of the light chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid.

In some aspects, the anti-Cx43 antibody or fragment thereof can comprise a heavy chain immunoglobulin variable region comprising: a complementarity determining region 1 (CDR1) comprising a sequence having at least 60% identity to a sequence set forth in SEQ ID NO: 19; a complementarity determining region 2 (CDR2) comprising a sequence having at least 60% identity to a sequence set forth in SEQ ID NO: 20; and/or a complementarity determining region 3 (CDR3) comprising a sequence having at least 60% identity to a sequence set forth in SEQ ID NO: 21. In some aspects, any one of the light chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid.

In some aspects, the anti-Cx43 antibody or fragment thereof can comprise a light chain immunoglobulin variable region comprising: a complementarity determining region 1 (CDR1) comprising a sequence having at least 60% identity to a sequence set forth in SEQ ID NO: 31; a complementarity determining region 2 (CDR2) comprising a sequence having at least 60% identity to a sequence set forth in SEQ ID NO: 32; and/or a complementarity determining region 3 (CDR3) comprising a sequence having at least 60% identity to a sequence set forth in SEQ ID NO: 33. In some aspects, any one of the light chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid.

In some aspects, the anti-Cx43 antibody or fragment thereof can comprise a heavy chain immunoglobulin variable region comprising: a complementarity determining region 1 (CDR1) comprising a sequence having a single amino acid change compared to a sequence set forth in SEQ ID NO: 19; a complementarity determining region 2 (CDR2) comprising a sequence having a single amino acid change compared to a sequence set forth in SEQ ID NO: 20; and/or a complementarity determining region 3 (CDR3) comprising a sequence having a single amino acid change compared to a sequence set forth in SEQ ID NO: 21. In some aspects, any one of the light chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid.

In some aspects, the anti-Cx43 antibody or fragment thereof can comprise a light chain immunoglobulin variable region comprising: a complementarity determining region 1 (CDR1) comprising a sequence having a single amino acid change compared to a sequence set forth in SEQ ID NO: 31; a complementarity determining region 2 (CDR2) comprising a sequence having a single amino acid change compared to a sequence set forth in SEQ ID NO: 32; and/or a complementarity determining region 3 (CDR3) comprising a sequence having a single amino acid change compared to a sequence set forth in SEQ ID NO: 33. In some aspects, any one of the light chain CDR1, CDR2 or CDR3 can comprises at least one amino acid substitution as compared to the parent CDR. In some aspects, the at least one amino acid substitution can be a cysteine residue to another amino acid. In some aspects, the at least one amino acid substitution can be a glycine residue to another amino acid.

Disclosed herein are methods of reducing or decreasing inflammatory gene expression. Disclosed herein are methods of reducing or decreasing inflammatory gene expression in a subject. In some aspects, the methods can comprise reducing or decreasing inflammatory gene expression in synovium macrophages. In some aspects, the methods can comprise reducing or decreasing inflammatory gene expression in fibroblasts. In some aspects, the expression of one or more inflammatory genes can be reduced or decreased. In some aspects, the one or more inflammatory genes can be MMP3/13, Cyclooxygenase 2 (COX2), ADAM metallopeptidase with thrombospondin type 1 motif 4 (Adamts4), or IL6.

In some aspects, the methods can comprise administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58. In some aspects, the methods can comprise administering to the subject a therapeutically effective amount of an anti-connexin 43 antibody or a fragment thereof, wherein the antibody or fragment thereof comprises a variable light chain comprising a sequence having at least 90% identity to a sequence set forth in SEQ ID NO: 60. In some aspects, the methods can comprise administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58; and a variable light chain comprising a sequence having at least 90% identity to a sequence set forth in SEQ ID NO: 60. In some aspects, the antibody or fragment thereof comprises a heavy chain immunoglobulin variable region comprising: a) a complementarity determining region 1 (CDR1) comprising the sequence of SEQ ID NO: 19 or a variant thereof; b) a complementarity determining region 2 (CDR2) comprising the sequence of SEQ ID NO: 20 or a variant thereof; and/or c) a complementarity determining region (CDR3) comprising the sequence of SEQ ID NO: 21 or a variant thereof. In some aspects, the antibody or fragment thereof comprises a light chain immunoglobulin variable region comprising: a) a complementarity determining region 1 (CDR1) comprising the sequence of SEQ ID NO: 31 or a variant thereof; b) a complementarity determining region 2 (CDR2) comprising the sequence of SEQ ID NO: 32 or a variant thereof; and/or c) a complementarity determining region 3 (CDR3) comprising the sequence of SEQ ID NO: 33 a variant thereof. In some aspects, any one of the CDR1, CDR2 or CDR3 of the heavy chain immunoglobulin variable region or the CDR1, CDR2, or CDR3 of the light chain immunoglobulin variable region can comprise at least one amino acid substitution as compared to the parent CDR. In some aspects, the at least one amino acid substitution is a cysteine residue to another amino acid or a glycine to another amino acid. In some aspects, the antibody or fragment thereof an comprise a heavy chain immunoglobulin variable region comprising: a) a complementarity determining region 1 (CDR1) comprising a sequence having at least 60% identity to a sequence set forth in SEQ ID NO: 19; b) a complementarity determining region 2 (CDR2) comprising a sequence having at least 60% identity to a sequence set forth in SEQ ID NO: 20; and/or c) a complementarity determining region 3 (CDR3) comprising a sequence having at least 60% identity to a sequence set forth in SEQ ID NO: 21. In some aspects, the antibody or fragment thereof can comprise a light chain immunoglobulin variable region comprising: a) a complementarity determining region 1 (CDR1) comprising a sequence having at least 60% identity to a sequence set forth in SEQ ID NO: 31; b) a complementarity determining region 2 (CDR2) comprising a sequence having at least 60% identity to a sequence set forth in SEQ ID NO: 32; and/or c) a complementarity determining region 3 (CDR3) comprising a sequence having at least 60% identity to a sequence set forth in SEQ ID NO: 33. In some aspects, the antibody or fragment thereof comprises: a) a heavy chain immunoglobulin variable region comprising: i) a first complementarity determining region 1 comprising a sequence having at least 60% identity to SEQ ID NO: 19; ii) a second complementarity determining region 2 comprising a sequence having at least 60% identity to SEQ ID NO: 20; and iii) a third complementarity determining region 3 comprising a sequence having at least 60% identity to SEQ ID NO: 21; and b) a light chain immunoglobulin variable region comprising: i) a first complementarity determining region 1 comprising a sequence having at least 60% identity to SEQ ID NO: 31; ii) a second complementarity determining region 1 comprising a sequence having at least 60% identity to SEQ ID NO: 32; and iii) a third complementarity determining region 1 comprising a sequence having at least 60% identity to SEQ ID NO: 33. In some aspects, the antibody or fragment thereof can comprise a heavy chain immunoglobulin variable region comprising: a) a complementarity determining region 1 (CDR1) comprising a sequence having a single amino acid change compared to a sequence set forth in SEQ ID NO: 19; b) a complementarity determining region 2 (CDR2) comprising a sequence having a single amino acid change compared to a sequence set forth in SEQ ID NO: 20; and/or c) a complementarity determining region 3 (CDR3) comprising a sequence having a single amino acid change compared to a sequence set forth in SEQ ID NO: 21. In some aspects, the antibody or fragment thereof can comprise a light chain immunoglobulin variable region comprising: a) a complementarity determining region 1 (CDR1) comprising a sequence having a single amino acid change compared to a sequence set forth in SEQ ID NO: 31; b) a complementarity determining region 2 (CDR2) comprising a sequence having a single amino acid change compared to a sequence set forth in SEQ ID NO: 32; and/or c) a complementarity determining region 3 (CDR3) comprising a sequence having a single amino acid change compared to a sequence set forth in SEQ ID NO: 33. In some aspects, the antibody or fragment thereof can comprise: a) a heavy chain immunoglobulin variable region comprising: i) a first complementarity region 1 comprising a sequence having a single amino acid change compared to SEQ ID NO: 19; ii) a second complementarity region 2 comprising a sequence having a single amino acid change compared to SEQ ID NO: 20; and iii) a third complementarity region 3 comprising a sequence having a single amino acid change compared to SEQ ID NO: 21; and b) a light chain immunoglobulin variable region comprising: i) a first complementarity region 1 comprising a sequence having a single amino acid change compared to SEQ ID NO: 31; ii) a second complementarity region 1 comprising a sequence having a single amino acid change compared to SEQ ID NO: 32; and iii) a third complementarity region 1 comprising a sequence having a single amino acid change compared to SEQ ID NO: 33.

In some aspects, any of the methods disclosed herein can comprise administering to the subject an effective amount of an expression vector encoding an anti-Cx43 antibody or fragment thereof. In some aspects, the anti-Cx43 antibody or fragment thereof can be administered in a pharmaceutically acceptable composition. In some aspects, the pharmaceutical composition can be lyophilized. In some aspects, the anti-Cx43 antibody or fragment thereof can be administered systemically. In some aspects, the anti-Cx43 antibody or fragment thereof can be administered intravenously, intradermally, intratumorally, intramuscularly, intraperitoneally, subcutaneously, or locally. In some aspects, the anti-Cx43 antibody or fragment thereof can be a humanized antibody or humanized fragment thereof. In some aspects, the anti-Cx43 antibody can be an IgG, IgM, IgA, IgD, IgE, or a genetically modified IgG class antibody comprising a first VH CDR corresponding to SEQ ID NO: 19, a second VH CDR corresponding to SEQ ID NO: 20, a third VH CDR corresponding to SEQ ID NO: 21, a first VL CDR corresponding to SEQ ID NO: 31, a second VL CDR corresponding to SEQ ID NO: 32, and a third VL CDR corresponding to SEQ ID NO: 33. In some aspects, the antibody can be an IgG class of antibody, wherein the IgG class antibody is an IgG1, IgG2, IgG3, or IgG4 class antibody.

In some aspects, any of the methods disclosed herein can further comprise administering at least a second therapeutic agent or second therapy to the subject. In some aspects, the second therapeutic agent can be a nonsteroidal anti-inflammatory drugs (NSAIDs), glucosamine, chondroitin supplements or a combination thereof. In some aspects, the second therapy can be a joint replacement surgery. In some aspects, a second therapeutic agent and a second therapy can be administered to the subject.

In some aspects, in any of the methods disclosed herein the anti-Cx43 antibody or fragment thereof can bind to a Cx43 hemichannel. In some aspects, in any of the methods disclosed herein the anti-Cx43 antibody or fragment thereof can inhibit or block the opening of a Cx43 hemichannel.

In some aspects, the anti-Cx43 antibody or fragment thereof can further comprise a tag sequence.

In some aspects, the anti-Cx43 antibody or fragment thereof can be a Fab fragment an Fab′ fragment or an F(ab′)2 fragment.

In some aspects, the one or more symptoms of osteoarthritis can be pain. Examples of symptoms of osteoarthritis include but are not limited to joint stiffness, decreased range of motion (flexibility) and swelling.

In some aspects, the one or more symptoms of rheumatoid arthritis can be pain. Examples of symptoms of rheumatoid arthritis also include but are not limited to joint stiffness, tenderness, weight loss, fever, fatigue or tiredness, and weakness.

In some aspects, the tendon or joint disorder can be juvenile arthritis, tendonitis, tendinosis, paratenonitis, tenosynovitis, gout or fibromyalgia. In some aspects, the one or more symptoms of a tendon disorder or a tendon injury can be pain. Examples of symptoms of a tendon disorder or a tendon injury include but are not limited to swelling, stiffness, tenderness, redness, and restricted joint movement.

In some aspects, the one or more symptoms of subchondral bone sclerosis can be pain. Examples of symptoms of subchondral bone sclerosis include but are not limited to stiffness and loss of flexibility, a grating feeling, hard bumps or bone spurs.

Treatment of Diseases

Disclosed herein are antibodies and fragments thereof that can be used to treat or prevent osteoarthritis, rheumatoid arthritis, a tendon disorder or a tendon injury, or subchondral bone sclerosis in a subject. Inhibiting or blocking the signaling of Cx43 hemichannels can be achieved by any suitable drug or therapeutic agent to reduce MMP13 or collagen X levels, cartilage degradation, and/or or synovial inflammation. In some aspects, the drug or therapeutic agent can be an anti-Cx43 antibody.

The compositions described herein can be administered to the subject (e.g., a human patient) in an amount sufficient to delay, reduce, or preferably prevent the onset of clinical disease. Accordingly, in some aspects, the patient can be a human patient. In therapeutic applications, compositions can be administered to a subject (e.g., a human patient) already with or diagnosed with osteoarthritis, rheumatoid arthritis, a tendon disorder or a tendon injury, or subchondral bone sclerosis or one or more symptoms of osteoarthritis, rheumatoid arthritis, a tendon disorder or a tendon injury, or subchondral bone sclerosis in an amount sufficient to at least partially improve a sign or symptom or to inhibit the progression of (and preferably arrest) the symptoms of the condition, its complications, and consequences. An amount adequate to accomplish this is defined as a “therapeutically effective amount.” A therapeutically effective amount of a composition (e.g., a pharmaceutical composition) can be an amount that achieves a cure, but that outcome is only one among several that can be achieved. As noted, a therapeutically effective amount includes amounts that provide a treatment in which the onset or progression of the disease, disorder, condition or injury is delayed, hindered, or prevented, or the disease, disorder, condition or injury or a symptom of the disease, disorder, condition or injury is ameliorated or its frequency can be reduced. One or more of the symptoms can be less severe. Recovery can be accelerated in an individual who has been treated. For example, treatment of osteoarthritis, rheumatoid arthritis, a tendon disorder or a tendon injury, or subchondral bone sclerosis may involve, for example, a reduction MMP13 levels, a reduction in collagen X levels, a reduction in cartilage degradation, a reduction of synovial inflammation or a reduction or prevention of pain.

In some aspects, the joint or tendon disorder can be juvenile arthritis, tendonitis, tendinosis, paratenonitis, tenosynovitis, gout, or fibromyalgia.

Osteoarthritis occurs when the protective cartilage that cushions the ends of bones wears down over time. In some aspects, osteoarthritis can damage or effect any joint. In some aspects, the joint can be in the hands, knees, hips, spine, feet, neck or shoulder.

Rheumatoid arthritis can be an autoimmune condition. In some aspects, rheumatoid arthritis can damage or effect any tissue and/or any joint. In some aspects, the tissue can be a joint. In some aspects, the joint can be in the hands, feet, knees, hips, shoulders and elbows. In some aspects, the rheumatoid arthritis can damage a wide variety of body systems. In some aspects, rheumatoid arthritis can damage or effect the skin, eyes, lungs, heart and blood vessels.

Subchondral bone sclerosis is the hardening of the bone just below the cartilage surface, and often appears in the later stages of osteoarthritis. In some aspects, subchondral bone sclerosis can be found at the load-bearing joints. In some aspects, subchondral bone sclerosis can be found in the knees, hips, hands, feet or spine.

Disclosed herein, are methods of treating a patient with arthritis or preventing the degeneration of cartilage associated with arthritis or subchondral bone sclerosis. In some aspects, the arthritis can be osteoarthritis or rheumatoid arthritis. Also disclosed herein, are methods of treating or preventing a tendon disorder or a tendon injury or subchondral bone sclerosis in a subject. In some aspects, the subject has been diagnosed with arthritis, osteoarthritis, rheumatoid arthritis, subchondral bone sclerosis or a tendon disorder or a tendon injury prior to the administering step.

The compositions described herein can be formulated to include a therapeutically effective amount of the antibodies or fragments thereof disclosed herein. In some aspects, antibodies or fragments thereof disclosed herein can be contained within a pharmaceutical formulation. In some aspects, the pharmaceutical formulation can be a unit dosage formulation.

The therapeutically effective amount or dosage of any of the anti-Cx43 antibodies or fragments thereof used in the methods as disclosed herein applied to mammals (e.g., humans) can be determined by one of ordinary skill in the art with consideration of individual differences in age, weight, sex, the severity of the subject's symptoms, and the particular composition or route of administration selected, other drugs administered and the judgment of the attending clinician. Variations in the needed dosage may be expected. Variations in dosage levels can be adjusted using standard empirical routes for optimization. The particular dosage of a pharmaceutical composition to be administered to the patient will depend on a variety of considerations (e.g., the severity of the cancer symptoms), the age and physical characteristics of the subject and other considerations known to those of ordinary skill in the art. Dosages can be established using clinical approaches known to one of ordinary skill in the art. A therapeutically effective dosage of an anti-hemichannel antibody can result in a decrease in severity of one or more disease symptoms, an increase in frequency and duration of disease symptom-free periods, or a prevention of impairment or disability due to the disease affliction. A therapeutically effective amount of a therapeutic compound or antibody can decrease MMP13 levels or collagen X levels, decrease cartilage degradation, decrease synovial inflammation, or otherwise ameliorate symptoms in a subject.

The duration of treatment with any composition provided herein can be any length of time from as short as one day to as long as the life span of the host (e.g., many years). For example, the compositions can be administered once a week (for, for example, 4 weeks to many months or years); once a month (for, for example, three to twelve months or for many years); or once a year for a period of 5 years, ten years, or longer. It is also noted that the frequency of treatment can be variable. For example, the present compositions can be administered once (or twice, three times, etc.) daily, weekly, monthly, or yearly.

The total effective amount of the antibodies or compositions as disclosed herein can be administered to a subject as a single dose, either as a bolus or by infusion over a relatively short period of time, or can be administered using a fractionated treatment protocol in which multiple doses are administered over a more prolonged period of time. Alternatively, continuous intravenous infusions sufficient to maintain therapeutically effective concentrations in the blood are also within the scope of the present disclosure.

The anti-Cx43 antibodies, fragments thereof or compositions described herein can be administered in conjunction with other therapeutic modalities to a subject in need of therapy. The present compounds can be given to prior to, simultaneously with or after treatment with other agents or regimes. For example, the anti-Cx43 antibodies disclosed herein can be administered alone or in conjunction with standard therapies used to treat arthritis, osteoarthritis, rheumatoid arthritis, subchondral bone sclerosis or a tendon disorder or a tendon injury. In some aspects, any of the anti-Cx43 antibodies or compositions described herein can be administered or used together with nonsteroidal anti-inflammatory drugs (NSAIDs), glucosamine and chondroitin supplements, joint replacement surgery or a combination thereof.

Pharmaceutical Compositions

Disclosed herein are compositions, e.g., pharmaceutical compositions, comprising one or a combination of monoclonal antibodies, or antigen-binding portion(s), or fragments thereof formulated with a pharmaceutically acceptable carrier. Such compositions may include one or a combination of (e.g., two or more different) antibodies, antibody fragments or immunoconjugates described herein. For example, a pharmaceutical composition described herein can comprise a combination of antibodies or antibody fragments thereof that bind to different epitopes on the target antigen or that have complementary activities.

Disclosed herein are pharmaceutical compositions comprising any of the anti-Cx43 antibodies or fragments thereof as described herein with a pharmaceutically acceptable carrier. Also disclosed herein are anti-Cx43 antibodies or pharmaceutical compositions for use as a medicament or for use in therapy for treating or preventing osteoarthritis, rheumatoid arthritis, a tendon disorder or a tendon injury, subchondral bone sclerosis, reducing MMP13 or collagen X levels, reducing cartilage degradation or reducing synovial inflammation.

Pharmaceutical compositions of the invention also can be administered as combination therapy, i.e., combined with other agents. For example, the combination therapy can include an anti-hemichannel antibody combined with at least one other therapeutic agent or therapy.

As used herein, the phrase “pharmaceutically acceptable carrier” includes any solvents, dispersion media, coatings, antibacterial and antifungal agents, isotonic and absorption delaying agents, and the like that are physiologically compatible. Preferably, the carrier can be suitable for intravenous, intramuscular, subcutaneous, or parenteral administration (e.g., by injection or infusion). Depending on the route of administration, the active compound, i.e., antibody, or immunoconjugate, may be coated in a material to protect the compound from the action of acids and other natural conditions that may inactivate the compound.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Pharmaceutically acceptable carriers include sterile aqueous solutions or dispersions and sterile powders for the extemporaneous preparation of sterile injectable solutions or dispersion. The use of such media and agents for pharmaceutically active substances is known in the art. Except insofar as any conventional media or agent is incompatible with the active compound, use thereof in the pharmaceutical compositions of the invention is contemplated. Supplementary active compounds can also be incorporated into the compositions.

Therapeutic compositions typically must be sterile and stable under the conditions of manufacture and storage. The composition can be formulated as a solution, microemulsion, liposome, or other ordered structure suitable to high drug concentration. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

Sterile injectable solutions can be prepared by incorporating the active compound in the required amount in an appropriate solvent with one or a combination of ingredients enumerated herein, as required, followed by sterilization microfiltration. Generally, dispersions are prepared by incorporating the active compound into a sterile vehicle that contains a basic dispersion medium and the required other ingredients from those enumerated herein. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred percent, this amount will range from about 0.01 percent to about ninety-nine percent of active ingredient, preferably from about 0.1 percent to about 70 percent, most preferably from about 1 percent to about 30 percent of active ingredient in combination with a pharmaceutically acceptable carrier.

Dosage regimens are adjusted to provide the desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of the antibody, the dosage ranges from about 0.0001 to 100 mg/kg, and more usually 0.01 to 5 mg/kg, 5 mg/kg to 10 mg/kg, 10 mg/kg to 15 mg/kg, 15 mg/kg to 20 mg/kg or 20 mg/kg to 25 mg/kg of the host body weight. In some aspects, the dosages can be 0.3 mg/kg body weight, 1 mg/kg body weight, 3 mg/kg body weight, 5 mg/kg body weight or 10 mg/kg body weight or within the range of 1-10 mg/kg. In some aspects, the dosages can be 0.3 mg/kg body weight, 1 mg/kg body weight, 3 mg/kg body weight, 5 mg/kg body weight, 10 mg/kg body weight, 15 mg/kg body weight, 20 mg/kg body weight, 25 mg/kg body weight or 30 mg/kg body weight or within the range of 1-30 mg/kg. In some aspects, the dosages can be about 15, 16, 17, 18, 19, 20, 21, 22, 23, 24 or 25 mg/kg body weight. In some aspects, the dosages can be 5 mg/kg body weight. In some aspects, the dosages can be 15 mg/kg body weight. In some aspects, the dosages can be 20 mg/kg body weight. In some aspects, the dosages can be 25 mg/kg body weight. An exemplary treatment regime entails administration once per week, once every two weeks, once every three weeks, once every four weeks, once a month, once every 3 months or once every three to 6 months. Preferred dosage regimens for an anti-hemichannel antibody of the invention include 1 mg/kg body weight or 3 mg/kg body weight via intravenous administration, with the antibody being given using one of the following dosing schedules: (i) every four weeks for six dosages, then every three months; (ii) every three weeks; (iii) 3 mg/kg body weight once followed by 1 mg/kg body weight every three weeks.

In some methods, two or more monoclonal antibodies with different binding specificities are administered simultaneously, in which case the dosage of each antibody administered falls within the ranges indicated. Antibody is usually administered on multiple occasions. Intervals between single dosages can be, for example, weekly, monthly, every three months or yearly. Intervals can also be irregular as indicated by measuring blood levels of antibody to the target antigen in the patient. In some methods, dosage is adjusted to achieve a plasma antibody concentration of about 1-1000 μg/ml and in some methods about 25-300 μg/ml.

Actual dosage levels of the active ingredients in the pharmaceutical compositions of the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions disclosed herein employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

Any of the compositions disclosed herein can be administered via one or more routes of administration using one or more of a variety of methods known in the art. As will be appreciated by the skilled artisan, the route and/or mode of administration will vary depending upon the desired results. In some aspects, the routes of administration for antibodies disclosed herein include but are not limited to intravenous, intramuscular, intradermal, intraperitoneal, subcutaneous, or other parenteral routes of administration, for example by injection or infusion. The phrase “parenteral administration” as used herein means modes of administration other than enteral and topical administration, usually by injection, and includes, without limitation, intravenous, intramuscular, intraarterial, intraperitoneal, transtracheal, subcutaneous, subcuticular, intraarticular injection and infusion.

Combination Treatments. The compositions and methods described herein can involve an antibody or an antibody fragment thereof against Cx43 (e.g., anti-Cx43 antibodies or fragments thereof) to inhibit or block the opening of the Cx43 hemichannel to, for example, reducing MMP13 or collagen X levels, protect, reduce or prevent degradation of cartilage, reduce synovial inflammation, reduce or ameliorate one or more symptoms of osteoarthritis, rheumatoid arthritis, subchondral bone sclerosis or a tendon disorder or a tendon injury, in combination with a second or additional therapeutic agent or therapy. Such therapy can be applied in the treatment of any disease that is associated with Cx43-mediated synovial inflammation, cartilage degradation, or increased levels of MMP13 or collagen X. For example, the disease may be osteoarthritis, rheumatoid arthritis, subchondral bone sclerosis or a tendon disorder or a tendon injury.

The methods and compositions, including combination therapies, enhance the therapeutic or protective effect, and/or increase the therapeutic effect of another anti-inflammatory agent, therapeutic agent or therapy. Therapeutic and prophylactic methods and compositions can be provided in a combined amount effective to achieve the desired effect, such as reducing MMP13 or collagen X levels, reducing cartilage degradation, reducing synovial inflammation and/or reducing or ameliorating one or more symptoms of osteoarthritis, rheumatoid arthritis, subchondral bone sclerosis or a tendon disorder or a tendon injury. This process may involve contacting the cells with both an antibody or antibody fragment and a second therapy. A tissue, tumor, or cell can be contacted with one or more compositions or pharmacological formulation(s) comprising one or more of the agents (i.e., antibody or antibody fragment or a second therapeutic agent), or by contacting the tissue, tumor, and/or cell with two or more distinct compositions or formulations, wherein one composition provides 1) an antibody or antibody fragment, 2) a second therapeutic agent, or 3) both an antibody or antibody fragment and a second therapeutic agent. Also, it is contemplated that such a combination therapy can be used in conjunction with surgical therapy, for example, joint replacement surgery.

The terms “contacted” and “exposed,” when applied to a cell, are used herein to describe the process by which a therapeutic construct and a second therapeutic agent are delivered to a target cell or are placed in direct juxtaposition with the target cell. To achieve the desired outcome, for example, both agents can be delivered to a cell in a combined amount effective to the desired outcome.

The anti-Cx43 antibodies or fragments thereof can be administered before, during, after, or in various combinations relative to a second therapeutic agent or therapy. The administrations may be in intervals ranging from concurrently to minutes to days to weeks. In aspects where the antibody or antibody fragment is provided to a patient separately from a second therapeutic agent or therapy, one would generally ensure that a significant period of time did not expire between the time of each delivery, such that the two compounds would still be able to exert an advantageously combined effect on the patient. In such instances, it is contemplated that one may provide a patient with the antibody therapy and the second therapeutic agent or therapy within about 12 to 24 or 72 h of each other and, more particularly, within about 6-12 h of each other. In some situations it may be desirable to extend the time period for treatment significantly where several days (2, 3, 4, 5, 6, or 7) to several weeks (1, 2, 3, 4, 5, 6, 7, or 8) lapse between respective administrations.

In some aspects, a course of treatment can last between 1-90 days or more (this such range includes intervening days). It is contemplated that one agent may be given on any day of day 1 to day 90 (this such range includes intervening days) or any combination thereof, and another agent is given on any day of day 1 to day 90 (this such range includes intervening days) or any combination thereof. Within a single day (24-hour period), the patient may be given one or multiple administrations of the agent(s). Moreover, after a course of treatment, it is contemplated that there can be a period of time at which no a second therapeutic agent or therapy is administered. This time period may last 1-7 days, and/or 1-5 weeks, and/or 1-12 months or more (this such range includes intervening days), depending on the condition of the patient, such as their prognosis, strength, health, etc. It is expected that the treatment cycles would be repeated as necessary.

In some aspects, anti-Cx43 antibodies or fragments thereof disclosed herein can be administered 1, 2, 3, 4, 5, 6, 7, 8, 9, 10 or more days after surgery. In some aspects, the surgery can be destabilization of the medial meniscus surgery. In some aspects, the surgery can be a type of surgery for the treatment of osteoarthritis. In some aspects, the surgery can be a cartilage replacement surgery (e.g., artificial endoprosthesis) or joint arthroplasty. In some aspects, the surgery can be a tendon repair surgery. In some aspects, the surgery can be associated with treating rheumatoid arthritis. In some aspects, the surgery can be a joint replacement, arthrodesis or a synovectomy. In some aspects, the anti-Cx43 antibodies or fragments thereof disclosed herein can be administered one or more times separated by one or more days. In some aspects, a second, third, fourth, fifth, and so on, administration of the anti-Cx43 antibodies or fragments thereof disclosed herein can be separated by 1, 2, 3, 4, 5, 6, or 7 days. In some aspects, the anti-Cx43 antibodies or fragments thereof disclosed herein can be administered one or more times separated by one or more weeks. In some aspects, a second, third, fourth, fifth, and so on, administration of the anti-Cx43 antibodies or fragments thereof disclosed herein can be separated by 1, 2, 3, 4, 5, 6, 7, 8, 9, 10, 11, or 12 weeks. In some aspects, the anti-Cx43 antibodies or fragments thereof disclosed herein can be administered one or more times separated by one or more months. In some aspects, a second, third, fourth, fifth, and so on, administration of the anti-Cx43 antibodies or fragments thereof disclosed herein can be separated by 1, 2, 3, 4, 5, 6, 7, 8, 9, 10, 11 or 12 months. In some aspects, the anti-Cx43 antibodies or fragments thereof disclosed herein can be administered one or more times separated by a variety of intervals, days, weeks, months or any combination thereof.

Various combinations may be employed. For the example below an antibody therapy is “A” and a second therapeutic agent or therapy is “B”:

A/B/A B/A/B B/B/A A/A/B A/B/B B/A/A A/B/B/B B/A/B/B B/B/B/A
B/B/A/B A/A/B/B A/B/A/B A/B/B/A B/B/A/A B/A/B/A B/A/A/B
A/A/A/B B/A/A/A A/B/A/A A/A/B/A.

Administration of any compound or therapy disclosed herein to a patient will follow general protocols for the administration of such compounds, taking into account the toxicity, if any, of the agents. Therefore, in some aspects there can be a step of monitoring toxicity that can be attributable to combination therapy.

Kits and Diagnostics

Disclosed herein are kits comprising one or more therapeutic agents and/or other therapeutic and delivery agents. In some aspects, the kit can be used for preparing and/or administering a therapy disclosed herein. The kit may comprise one or more sealed vials containing any of the pharmaceutical compositions disclosed herein. The kit may include, for example, at least one anti-Cx43 antibody or fragment thereof as well as reagents to prepare, formulate, and/or administer the components one or more of the compositions disclosed herein or perform one or more steps of the inventive methods. In some aspects, the kit may also comprise a suitable container, which can be a container that will not react with components of the kit, such as an eppendorf tube, an assay plate, a syringe, a bottle, or a tube. The container may be made from sterilizable materials such as plastic or glass.

The kit may further include an instruction sheet that outlines the procedural steps of the methods set forth herein, and will follow substantially the same procedures as described herein or are known to those of ordinary skill in the art. The instruction information may be in a computer readable media containing machine-readable instructions that, when executed using a computer, cause the display of a real or virtual procedure of delivering a pharmaceutically effective amount of a therapeutic agent.

### EXAMPLES

It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Inhibition of Osteoarthritis Progression by Blocking Connexin 43 Hemichannels

In primary mouse chondrocytes, ethidium bromide dye uptake assay indicated that the M1 antibody blocked the IL-1β treatment induced HCs opening. To investigate the function of Cx43 hemichannels in OA progression, post-traumatic OA model was induced in male C57BL/6 mice by surgically destabilization of the medial meniscus (DMM). The M1 antibody or saline was administered by intraperitoneal injection twice during 8 weeks post-surgery. Cartilage degradation and synovial inflammation were analyzed by histology and immunohistochemistry. Compared to saline group, M1 antibody treatment significantly improved cartilage integrity as quantified by the OARSI scoring system, decreased degree of synovitis, along with reduction of Mmp13 and Collagen X levels. Microcomputed tomography (μCT) of subchondral bone sclerosis showed a decrease in BV/TV, Tb.Th and an increase in Tb.Sp after inhibition of Cx43 hemichannels. In addition, Von Frey filament and open-field tests ameliorated OA associated pain symptoms in DMM mice after M1 antibody treatment, while no difference was observed between saline and antibody treated sham mice. The results also show that the M1 antibody was primarily localized to the synovium region, and the signals were detected even more than 2 weeks after M1 antibody administration. In vivo dye uptake and immunofluorescence assay further validated the role of the M1 antibody in blocking OA induced hemichannel opening in synoviocytes that express Cx43. Taken together, these results suggest that targeting Cx43 hemichannels by the M1 antibody is therapeutic strategy that can be used to mitigate the catabolic and inflammatory environment of the joint during OA progression. Due to the similar mechanism of action, M1 antibody therapy may be applicable to rheumatoid arthritis (RA) and other tendon disorders and injuries.

Materials and Methods. Animals and surgery model. The mice used were of C57BL/6 background. The mice were housed in a temperature-controlled room with a light/dark cycle of 12 hours under specific pathogen-free conditions. Food and water were provided ad libitum.

Destabilization of the medial meniscus (DMM) surgery was performed on the right knee joint of mice (Glasson S S, et al. Osteoarthritis and cartilage. 2007; 15(9):1061-9). Briefly, a 3 mm longitudinal incision over the distal patella to proximal tibial plateau was made. The joint capsule immediately medial to the patellar tendon was incised with a #15 blade and the joint capsule opened with micro-iris scissors. Blunt dissection of the fat pad over the intercondylar area was then performed to expose the intercondylar region, providing visualization of the meniscotibial ligament of the medial meniscus. Mild hemorrhage from the fat pad upon blunt dissection was controlled by pressure from absorption spears. Sectioning of medial meniscotibial ligament with micro-surgical knife, with the blade directed proximo-laterally, gave destabilization of the medial meniscus (DMM). Sham surgery was performed with the ligament visualized but not transected.

Histology. At 8 weeks after the induction of osteoarthritis (OA), the femur and tibia bones isolated from DMM and sham operated mice were fixed in 4% paraformaldehyde for 2 days prior to decalcification with 10% EDTA (pH 7.5) for 3 weeks. The samples were embedded in paraffin, and 5-μm-thick sections were collected on glass slides. Sections were deparaffinized in xylene, followed by a graded series of alcohol washes. To determine the extent of cartilage deterioration, sections were stained with Safranin O/Fast Green. OARSI scoring was performed to evaluate knee joint articular cartilage destruction (Glasson S S, et al. Osteoarthritis and cartilage. 2010; 18 Suppl 3:S17-23). Specifically, both medial femoral condyle and medial tibial plateau were analyzed through three-level sections of the joints and the severity of OA is expressed as the averaged and summed scores for the entire joint.

The degree of synovitis is determined considering synovial membrane thickening and synovial hyperplasia and scored on a scale of 0-3 (0, no synovitis; 1, mild synovitis; 2, moderate synovitis; and 3, severe synovitis) (Krenn V, et al. Pathology, research and practice. 2002; 198(5):317-25; and Huesa C, et al. Annals of the rheumatic diseases. 2016; 75(11):1989-97. Blinded histomorphometric assessment was performed.

Immunohistochemistry. The ABC (avidin-biotin-peroxidase complex) Immunostaining Assay Kit (Vector Laboratories, PK-6101) was used. Briefly, bone tissue sections were antigen unmasked using sodium citrate buffer (pH 6.0) at 65° C. for 2 h for MMP13 staining and trypsin digestion buffer (pH 7.8) at 37° C. for 30 min for collagen X staining. Bone tissue sections were then treated with goat normal serum for 20 min at room temperature to block nonspecific background staining. Tissue sections were stained with anti-MMP13 polyclonal antibody (1:200 dilution, Abcam, ab39012), or labeled with anti-collagen X monoclonal antibody (1:100 dilution, Thermo Fisher, 14-9771-82) at 4° C. overnight. Then, the sections were incubated for 30 min with biotin-labeled secondary antibody and VECTASTAIN ABC Reagent for 30 min Samples were washed in PBS buffer and developed in DAB (SK4100) chromogen solution (Vector Laboratories, Burlingame, CA, USA). Tissues were then counterstained with VECTOR Hematoxylin (H-3401) for 5 min at room temperature. Slides were then dehydrated, cleared, and mounted. Sections were photographed using the Keyence microscope (BZ-X710, Keyence, Osaka, Japan). The percentage of MMP13 or Col X-positive chondrocytes were quantified using NIH Image J software.

Immunofluorescent staining. The isolated femur and tibia bones were fixed in 4% paraformaldehyde for 2 days prior to decalcification with 10% EDTA (pH 7.5) for 3 weeks, and then dehydrated in 30% sucrose/PBS for 3 days. Samples were embedded in the Tissue-Tek CRYO OCT compound (Thermo Fisher Scientific, Pittsburgh, PA, USA), and 12-μm-thick sections were collected on glass slides. Sections were blocked and permeabilized with 2% goat serum, 2% fish skin gelatin, 0.025% Triton X-100 and 1% bovine serum albumin in PBS. The sections were incubated overnight at 4° C. with affinity-purified antibodies against Cx43 (CT) (1:100) (Cherian P P, et al. The Journal of biological chemistry. 2003; 278(44):43146-56), for 1 hour with the appropriate secondary antibody. Slides were mounted using Vectashield mounting medium (H-1000, Vector Lab). Confocal fluorescence imaging was performed using a confocal laser scanning microscope (LSM770, Carl Zeiss, Oberkochen, Germany).

Animal behavioral tests. The paw withdraw threshold was measured using von Frey filaments 0.007 to 2. Mice were placed in acrylic chambers (5.5×10 cm) suspended above a wire mesh grid and allowed to acclimatize to the testing apparatus for 30 minutes prior to experiments. When the mouse was not moving the von Frey filaments were pressed against the plantar surface of the paw until the filament buckled and held for a maximum of 3 seconds. A positive response was noted if the paw was sharply withdrawn on application of the filament. Flinching immediately upon removal of the filament was also considered a positive response (Chaplan S R, et al. Journal of neuroscience methods. 1994; 53(1):55-63; and Bonin R P, et al. Molecular pain. 2014; 10:26). Testing began with filament number 0.4 and progressed according to an up-down method.

Spontaneous locomotor activity was analyzed using the open-field test. Mice were placed into the center of a chamber 25×25×25 cm to allow free exploration. The experiments were performed for 10 minutes. Total travel distance, and immobility time were measured by computerized analysis. Mice behavior was recorded, and videos were analyzed by use of ANY-maze behavior tracking software (Stoelting Co., Wood Dale, IL, USA).

Micro-computed tomography. Following behavioral tests, mice were sacrificed and hindlimbs were dissected immediately and fixed in 4% paraformaldehyde. The structural properties of subchondral bone were evaluated using a 3D reconstruction of μCT imaging system (Brüker SkyScan 1173; Brüker microCT, Kontich, Belgium). Samples were scanned in saline with the following settings: 60 kV, 167 mA beam intensity, 0.5 mm aluminum filter, 0.7° rotation step, 4-frame averaging, 1090 ms integration time, 1024×1024 pixel matrix, and a 10 μm isotropic voxel dimension. After scanning, noise was removed from the images by eliminating disconnected objects smaller than 4 pixels in size. Morphometric analysis was performed on 50 slices extending proximally, beginning with the first slice in which the tibia condyles had fully merged. The subchondral bone was segmented from the cortical shell manually on key slices using a contouring tool, and the contours were morphed automatically to segment the trabecular bone on all slices. The morphometry was reconstructed and analyzed. A grayscale value of 95 in a set of 8-bit slices was set as the threshold and was applied to the specimens after comparing grayscale and binarized images in both groups. After thresholding, the BV/TV (%), Tb.Th (mm), Tb.Sp (mm), and Tb.N (mm⁻¹) were quantified.

In vivo dye uptake assay. Evans Blue dye (Mw: 900 Da) (200 mg/kg) and FITC-dextran (Mw: 10,000 Da) (200 mg/kg) were co-injected 4 days post-antibody treatment via the tail vein. One-hour post administration of the dye, mice were anesthetized, and cardiac perfusion was performed with cold PBS and 4% paraformaldehyde. Both hindlimbs were isolated and prepared for frozen sections. Sections were stained with DAPI and photographed using the Keyence microscope (BZ-X710, Keyence, Osaka, Japan). The intensity of dye uptake was quantified by NIH Image J software.

Statistical analysis. Statistical analysis was performed using GraphPad Prism5 statistics software (GraphPad). The data are presented as mean±SEM. T test and two-way analysis of variance with Tukey's test was used for statistical analysis. Asterisks indicate the degree of significant differences compared with the controls (*P<0.05; **P<0.01; ***P<0.001).

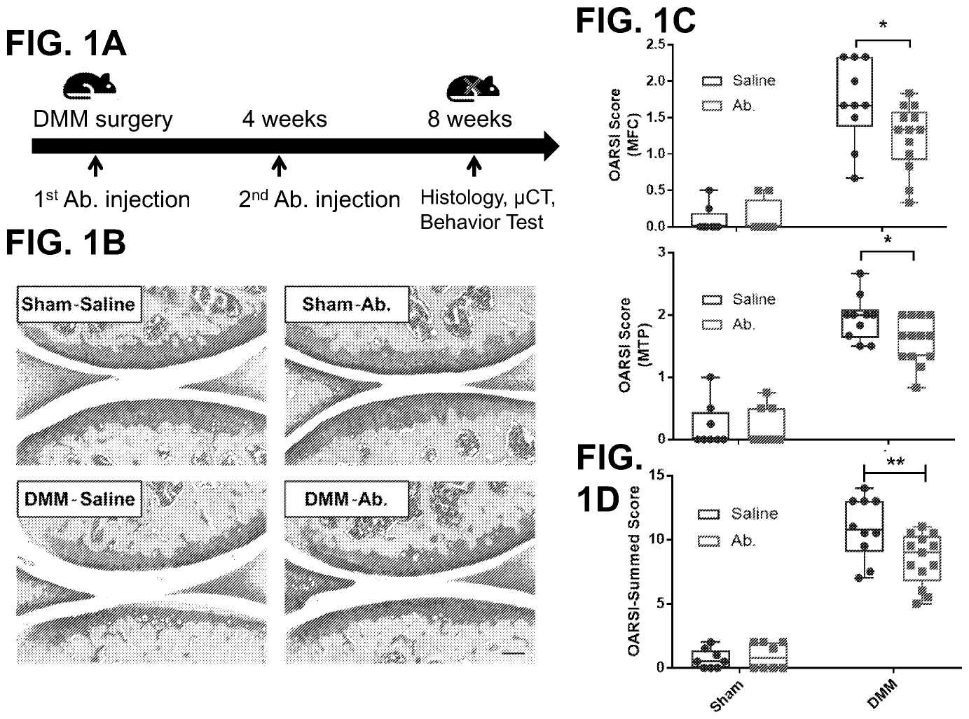

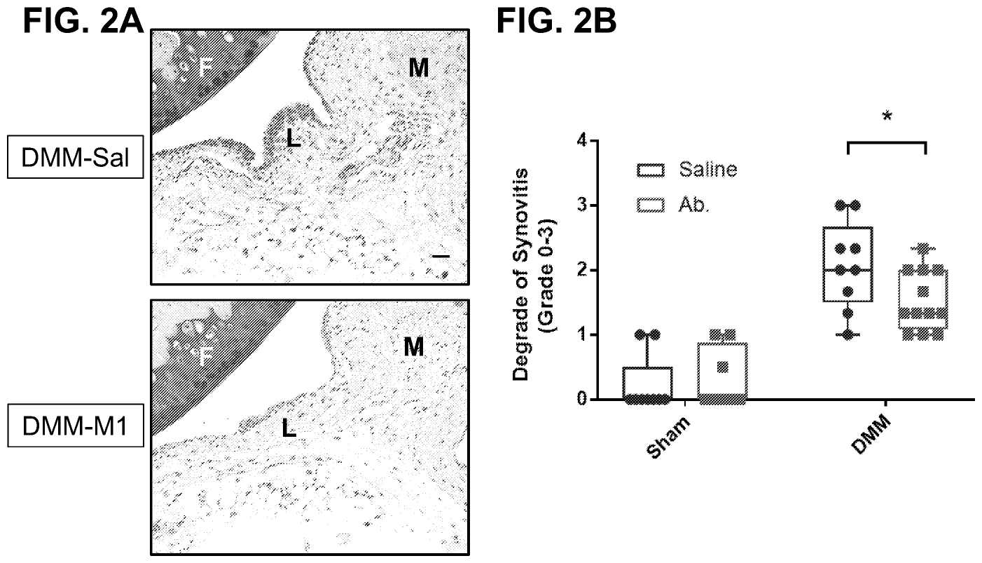

Results. Inhibition of Cx43 hemichannel ameliorates histopathological changes of OA. To investigate the function of Cx43 hemichannel in OA progression, post-traumatic OA model was induced in male C57BL/6 mice by surgically destabilization of the medial meniscus (DMM). The anti-Cx43 hemichannel blocking antibody (M1 antibody) or saline was administered by intraperitoneal injection twice at 30 minutes and four weeks post-surgery (FIG. 1A). Histological staining of Safranin O/Fast Green was utilized to visualize the severity of articular cartilage damage, which was determined using the Osteoarthritis Research Society International (OARSI) scoring system (Glasson S S, et al. Osteoarthritis and cartilage. 2010; 18 Suppl 3:S17-23) by two blinded observers (FIGS. 1B-D). At eight weeks after DMM induction of OA, mice exhibited OA pathological changes characterized by proteoglycan loss and cartilage erosion (FIG. 1B), with an average OARSI score of 1.95±0.11 and 1.72±0.18 in the medial tibial plateau (MTP) and medial femoral condyle (MFC), respectively. Compared to saline-treated group, M1 antibody treatment showed marked protection in the severity of cartilage degeneration and a significant reduction in the OARSI scores in both medial tibial plateau and medial femoral condyle (FIG. 1C). Sham-operated mice that received either saline or M1 antibody treatment did not exhibit pathologic changes in the articular cartilage (FIG. 1C). Summed OARSI score of MTP and MFC through three-level sections of the joints was also quantified, further validated the significantly improved structural integrity of the articular cartilage after M1 antibody treatment (FIG. 1D). Moreover, synovium hyperplasia was quantified by the synovitis score (Krenn V, et al. Pathology, research and practice. 2002; 198(5):317-25; and Huesa C, et al. Annals of the rheumatic diseases. 2016; 75(11):1989-97). Results showed a marked reduction in the degree of synovitis in the M1 antibody-treated group compared with the saline-treated group (FIGS. 2A and B).

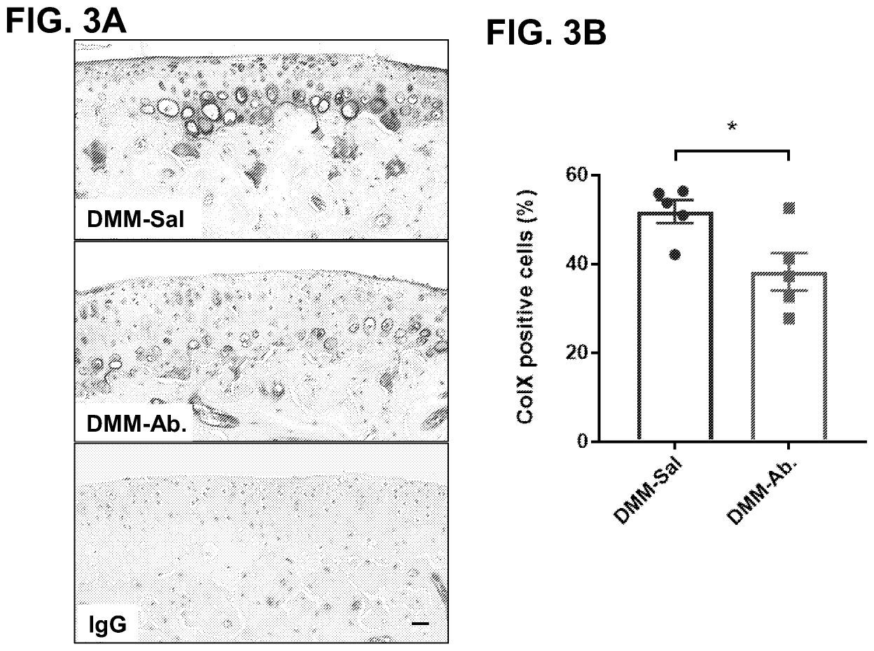

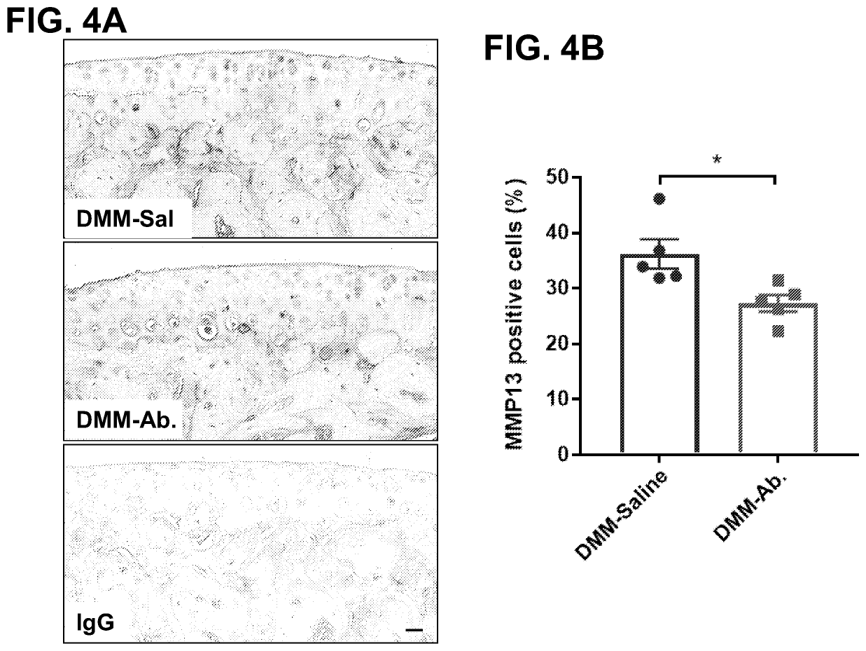

Inhibition of Cx43 hemichannel reduces matrix protein and protease levels induced by OA. Collagen X (Col X) is one of the most important markers of hypertrophic chondrocytes, and matrix metalloproteinase 13 (MMP13) is the most potent enzyme in cleaving type II collagen, the principal form in articular cartilage. It was tested whether the reduction of Col X and MMP13 could underlie the chondroprotective effect of the M1 antibody. Using immunohistochemistry, at eight weeks following DMM, the percentage of Col X positive cells was reduced from 52% in vehicle-treated mice to 38% in M1 antibody-treated mice (FIG. 3). Similarly, immunohistochemical staining of MMP13 was performed for analysis of the expression. Results showed that MMP13-positive chondrocytes in the saline-treated DMM mice were distributed in the three zones of the articular cartilage of the femoral and tibial condyles. In contrast, the MMP13-positive chondrocytes in M1 antibody-treated mice were localized mainly in the middle and deep zones (FIG. 4A). At eight weeks after DMM, the percentage of MMP-13-positive cells in the articular cartilage was reduced from 36% in saline-treated mice to 27% in M1 antibody-treated mice (FIG. 4B). Mouse or rabbit IgG was used as isotype control (FIG. 3A and FIG. 4A, lower panels), showing no reactivity.

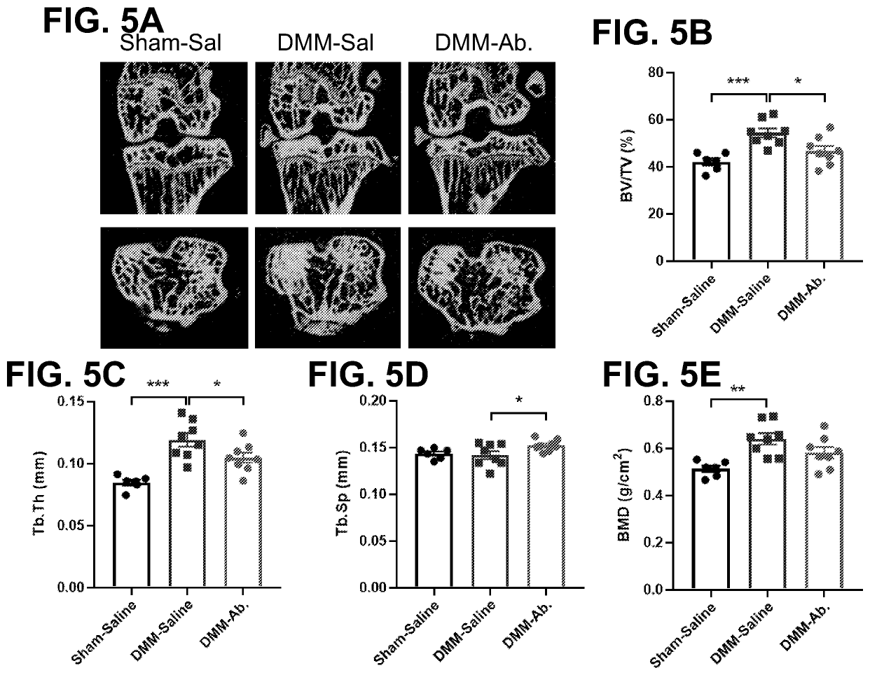

Inhibition of Cx43 hemichannel mitigates subchondral bone sclerosis of OA. As OA is also characterized by abnormal subchondral bone remodeling, to study the effects of inhibiting Cx43 hemichannels on subchondral bone microstructure, microCT analysis was performed. The 3D rendering and reconstruction scans of DMM mice at eight weeks post-surgery showed alterations of the knee joint, including the thickness of the medial subchondral plate, and pronounced sclerosis of the subchondral bone, compared to mice with sham surgery (FIG. 5A). Consistently, micro-CT analysis revealed an increased relative bone volume fraction (bone volume/total volume, BV/TV), trabecular thickness (Tb.Th) and bone mineral density (BMD) at the tibia subchondral bone of mice after DMM as compared to Sham surgery (FIGS. 5B, C and E). Compared to saline-treated DMM mice, the administration of anti-Cx43 antibody showed significantly decreased BV/TV, Tb.Th and BMD (FIGS. 5B, C and E), along with elevated trabecular separation (Tb.Sp) at the tibia subchondral bone (FIG. 5D), indicating that blocking Cx43 hemichannel mitigates subchondral bone sclerosis of OA.

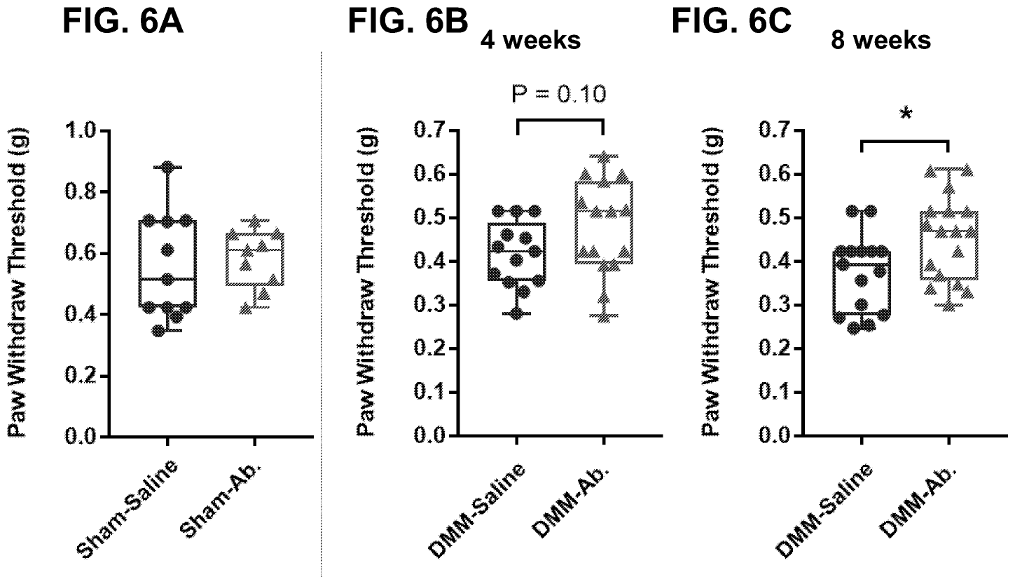

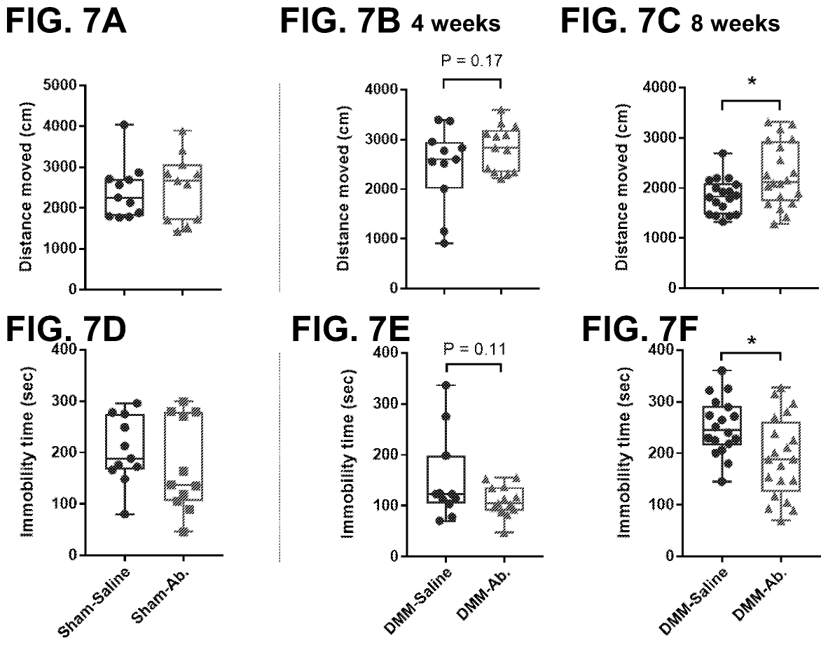

Inhibition of Cx43 hemichannel reduces OA-related pain symptoms. The progression of OA is accompanied by secondary clinical symptoms, most prominently pain (Felson D T. Arthritis research & therapy. 2009; 11(1):203). Mechanical allodynia was measured by withdrawal thresholds to von Frey filaments applied to the plantar surface of hind paws with sham or DMM surgery at two time points. At 4 weeks after DMM, compared to mice with saline treatment, inhibition of Cx43 hemichannel showed a slight trend of increased response threshold to mechanical stimuli (FIG. 6B). At 8 weeks after DMM, mice with M1 antibody treatment showed a paw withdrawal threshold 121.4% of that observed in saline treated DMM mice (FIG. 6C), implicating that they experience much less pain than the saline-treated group. In addition, locomotor activity was monitored by subjecting mice to an open field test to quantitate movement following sham or DMM surgery treated with saline/M1 antibody. At 4 weeks after DMM, there was a trend of increased distance moved and decreased immobility time in Cx43 antibody treated mice compared to saline group (FIGS. 7B and E). At 8 weeks after DMM, saline treated DMM mice exhibited reductions in distance traveled and elevations in immobility time compared to sham-operated mice, while M1 antibody treatment suppressed these pain responses (FIGS. 7C and F). No difference was observed between saline and M1 antibody treated sham mice at 8 weeks in regarding to paw withdraw threshold by von frey filament test (FIG. 6A), distance traveled and immobility time by open field test (FIGS. 7A and D).

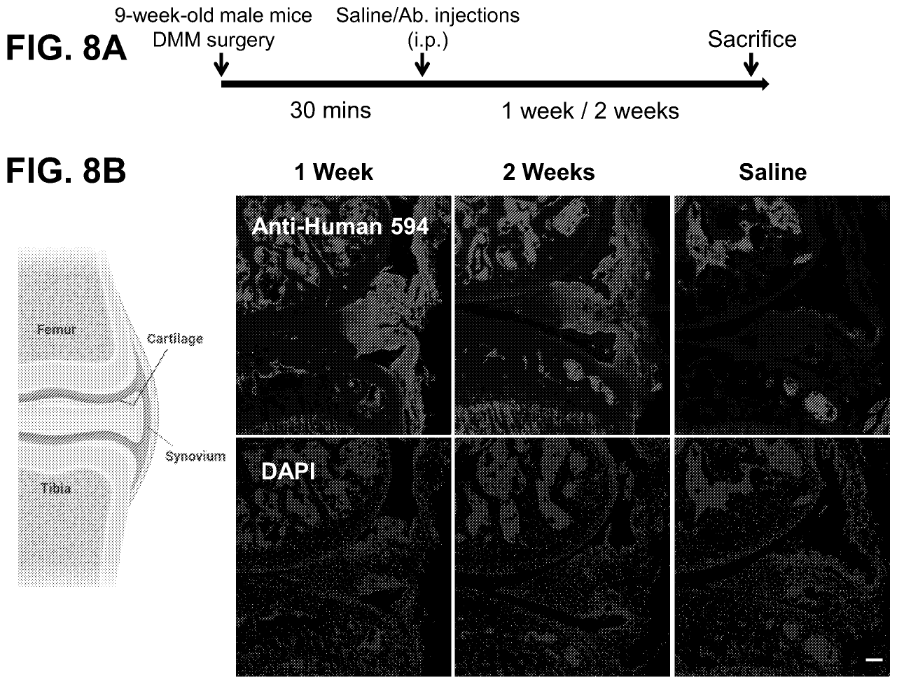

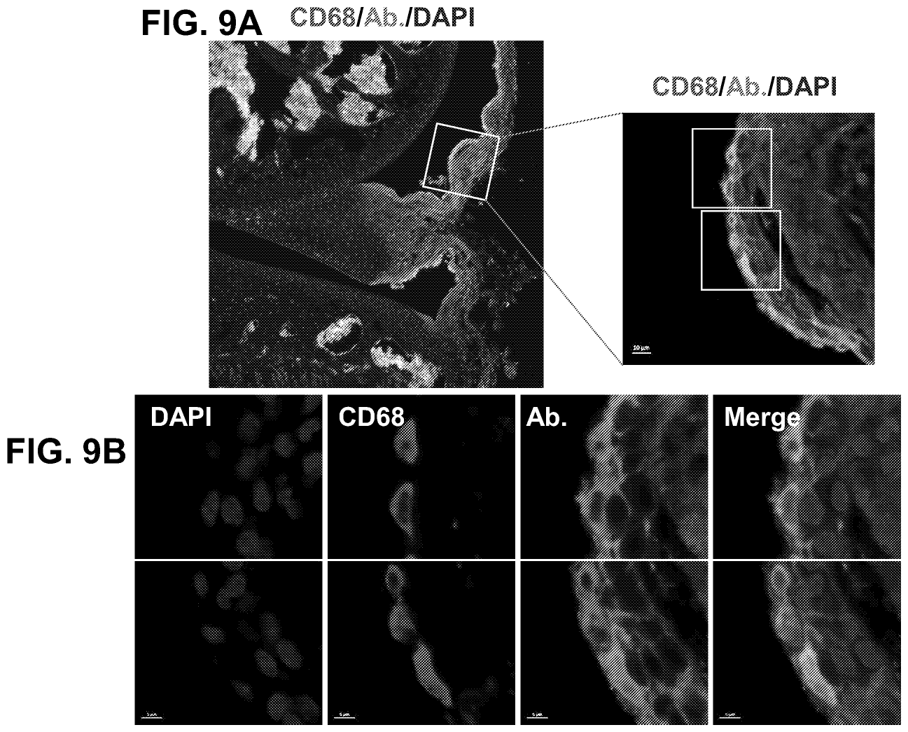

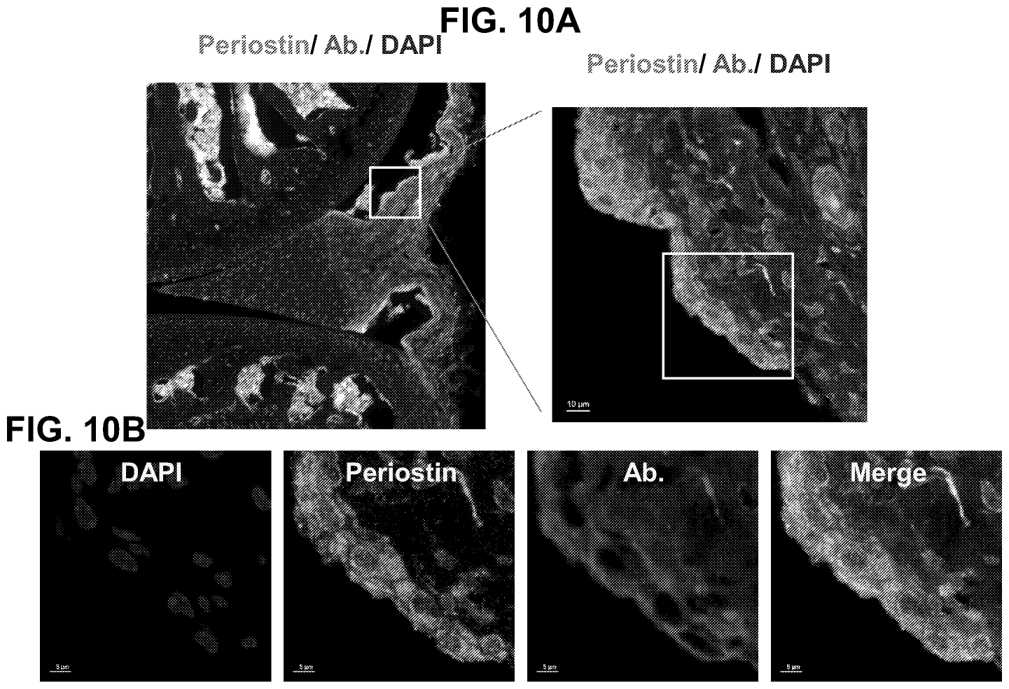

M1 antibody binds to synovium and inhibits hemichannel opening in OA. Due to the dense nature of cartilage and large size of antibodies, a widely held perception is that antibodies are unable to penetrate the cartilage matrix. To determine the antibody binding pattern in the knee joint region, M1 antibody (25 mg/kg) with human IgG epitope or saline was intraperitoneally (i.p.) injected at 30 mins after DMM surgery. As shown in the diagram (FIG. 8A), mice were euthanized and perfused before isolation of hindlimbs at one or two weeks after injection. Frozen tissue sections were prepared and immuno-labeled with Alexa Fluor 594-conjugated anti-human IgG secondary antibody. The signal was detectable at both one and two weeks after antibody administration, with the antibody binding site primarily localized in the synovium region (FIG. 8B). The synovium of joints comprises macrophage-like type A cells, which are responsible for phagocytosing synovial fluid, and fibroblast-like type B cells, which produce hyaluronic acid and mucin to nourish and mechanically protect, respectively, the underlying articular cartilage (Donahue H J, et al. Nature reviews Rheumatology. 2017; 14(1):42-51). Then the sections were immuno-labeled with the macrophage marker CD68, and the M1 antibody was co-localized with CD68 in the synovium region (FIG. 9). In addition, the co-localization of M1 antibody and synovial fibroblast marker Periostin was also observed (FIG. 10).

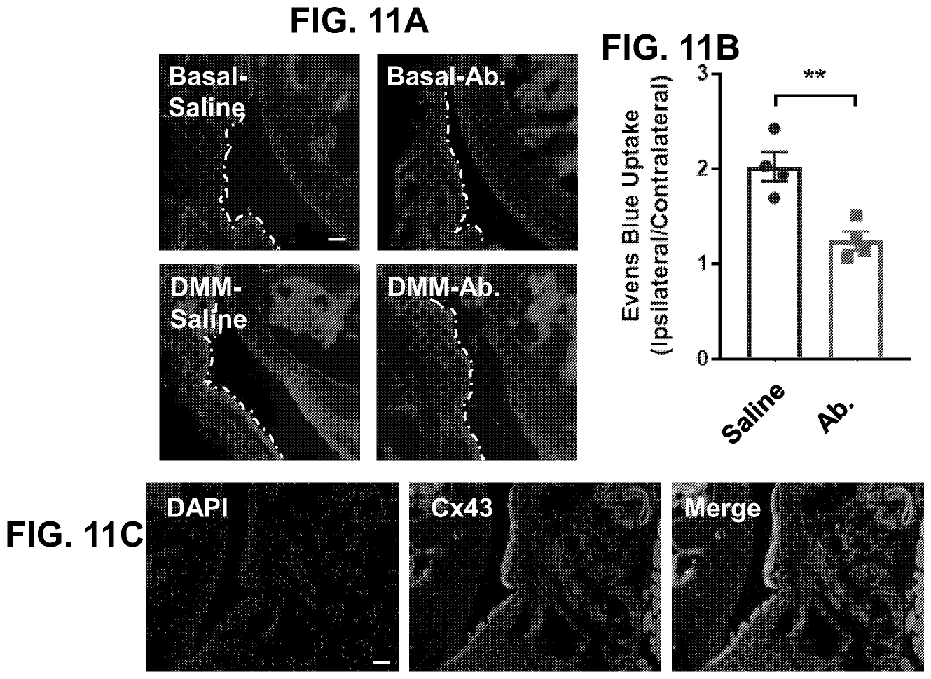

To determine the Cx43 hemichannel activity in vivo, Evans Blue dye uptake assay was performed. Mice were treated with saline or Cx43 antibody (M1 antibody) (25 mg/kg) at 30 mins after DMM surgery. Tail vein injection of Evans Blue dye (200 mg/kg) was performed at two weeks after the treatment, and Evans Blue dye uptake intensity was quantified in synovium region. Results showed that DMM increased hemichannel opening by 2 folds, which can be blocked by the M1 antibody administration (FIGS. 11A and B). Frozen sections prepared from saline-treated mice were immuno-labeled with the M1 antibody, further validated the role of the M1 antibody in blocking the osteoarthritis induced hemichannel opening in synoviocytes that express Cx43 (FIG. 11C).

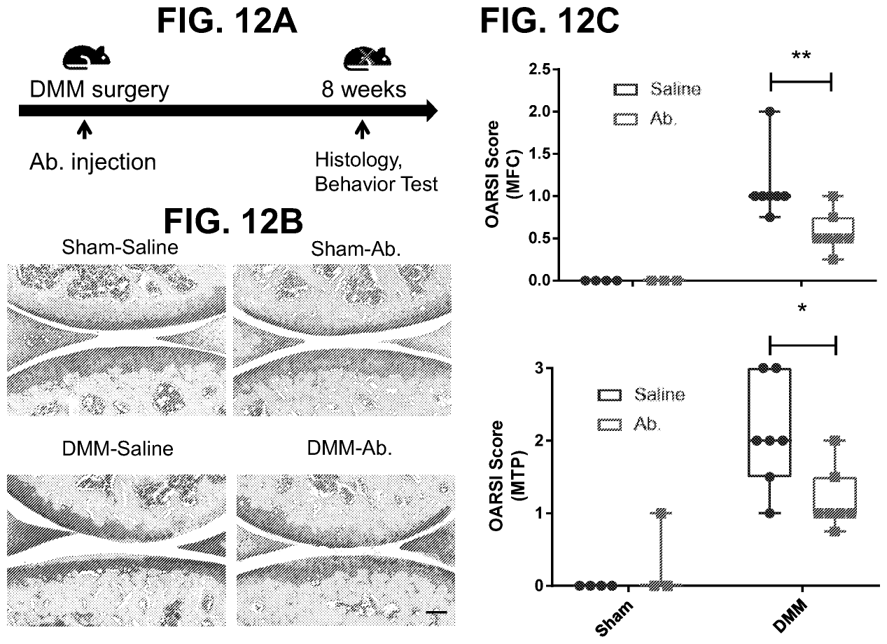

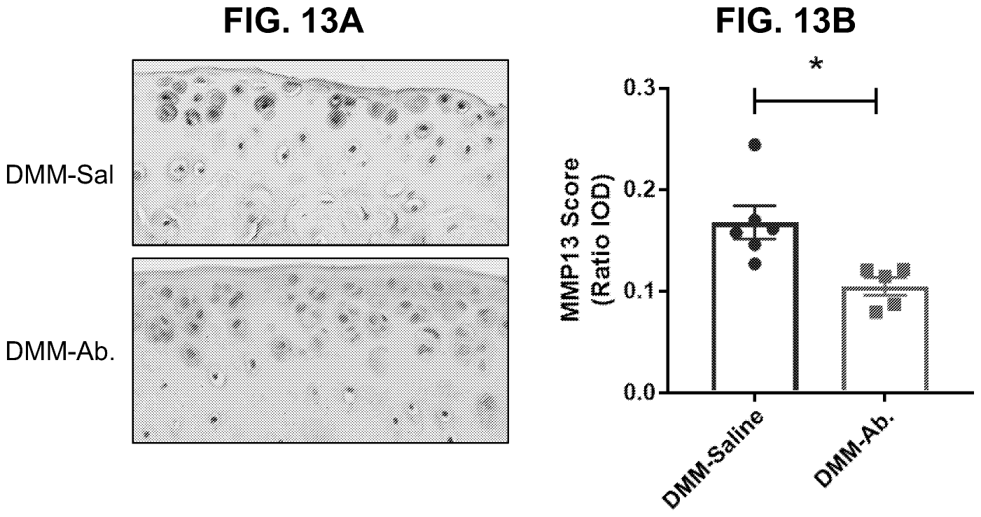

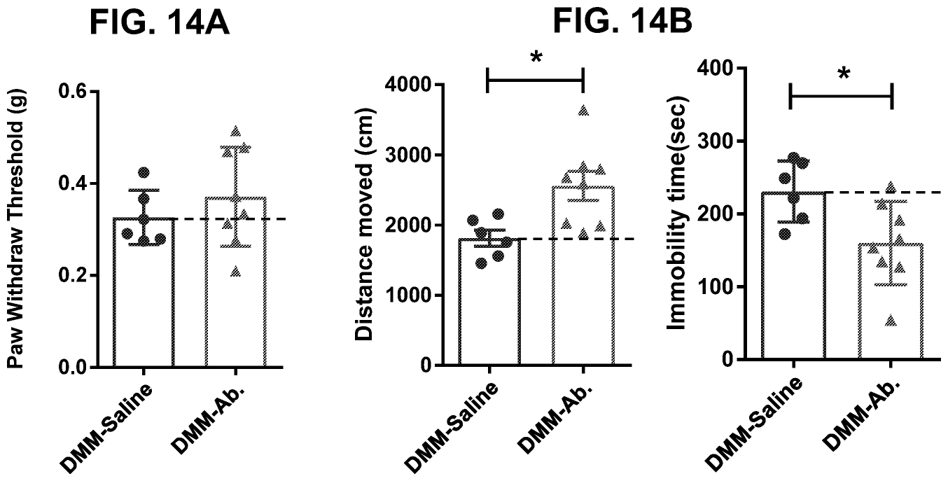

Single administration of anti-Cx43 hemichannel antibody (M1 antibody) ameliorates OA severity. It was investigated whether one-time administration of M1 antibody could show effects on slowing down OA progression. Anti-Cx43 hemichannel antibody (M1 antibody) or saline was administered by intraperitoneal injection just once at 30 minutes post-surgery, and behavior tests were performed before mice sacrificed for histology at eight weeks after DMM (FIG. 12A). Safranin O/Fast Green staining results indicated that there was less proteoglycan loss and cartilage erosion after M1 antibody treatment compared to saline control (FIG. 12B). The quantification of cartilage degeneration by OARSI scores showed significant reduction in the in both medial tibial plateau and medial femoral condyle in DMM mice with M1 antibody treatment (FIG. 12C). Moreover, the MMP13 score was determined as a ratio of integrated optical density (IOD) to articular cartilage area, which was reduced from 0.17±0.016 in saline-treated mice to 0.10±0.009 in M1 antibody treated mice (FIG. 13). At eight weeks after DMM, compared to mice with saline treatment, inhibition of Cx43 hemichannel showed a slight trend of increased paw withdraw threshold to mechanical stimuli (FIG. 14A). Consistently, there was a significant increase of distance traveled and reduction of immobility time in DMM mice with M1 antibody treatment compared to saline control (FIG. 14B).

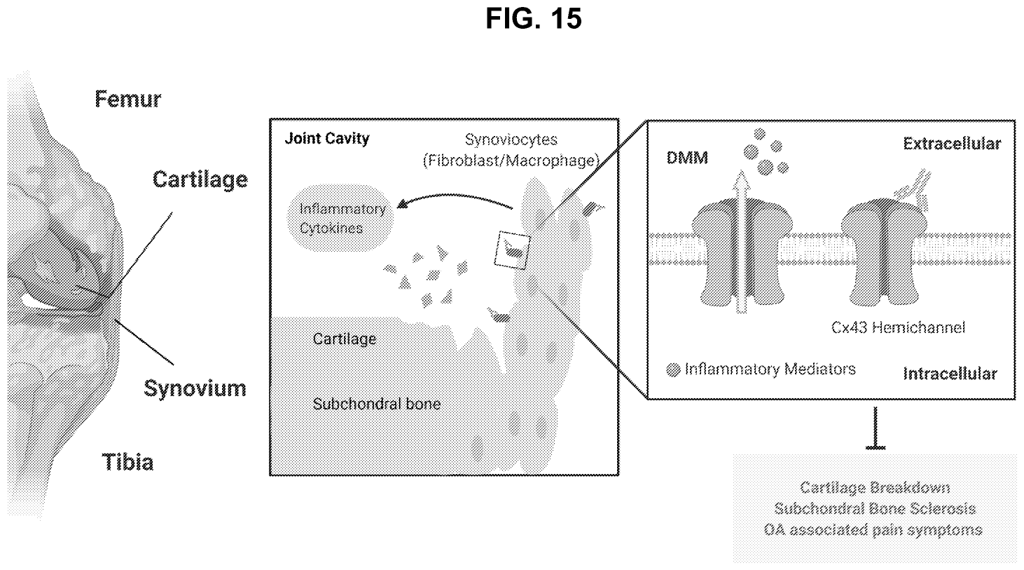

Conclusions. The results suggest that administration of the anti-Cx43 hemichannel antibody (M1 antibody) decreases articular cartilage degeneration, degree of synovitis and MMP13/ColX expression in DMM induced OA model. MicroCT analysis of subchondral bone sclerosis shows a decrease in BV/TV, Tb.Th and an increase in Tb.Sp after inhibition of Cx43 hemichannels. In addition, Von Frey filament and open-field tests exhibits ameliorated OA associated pain symptoms and improved mobility in antibody-treated DMM mice. In vivo dye uptake and immunofluorescence assay validated the role of anti-Cx43 hemichannel antibodies in blocking OA induced HCs opening in synoviocytes that express Cx43. Taken together, the results suggest that targeting Cx43 hemichannel is a therapeutic strategy that can be used to mitigate the catabolic and inflammatory environment of the joint during OA progression (FIG. 15).

### Example 2—Anti-Cx43 Hemichannel Antibody (M1 Antibody) Decreases Osteoarthritis Synovitis and Blocked Cx43 Hemichannel Opening and Inflammatory Gene Expression

Dye uptake assay. Cells were incubated with a mixture of 0.1 mM ethidium bromide (EtBr, MW 394 Da) and 1 mg/ml FITC-dextran (MW 10 kDa) for 5 minutes. EtBr was used as a tracer to detect hemichannel activity, and FITC-dextran, which is too large to pass through hemichannels but is taken up by dying cells, was used as a negative control. Cells were then rinsed five times with PBS, followed by fixing with 2% paraformaldehyde for 10 minutes. At least six microphotographs of fluorescence fields were captured under a 20× fluorescent microscope (Keyence, BZ-X710, Osaka, Japan). For each image, the average intensity of EtBr fluorescence was measured and quantified from at least 30 random cells using ImageJ software (NIH, Bethesda, USA). Experiments were repeated 3 times and the collected data were illustrated as pixel mean in arbitrary units.

RNA isolation and real-time PCR. Total RNA was isolated from RAW264.7 and SW982 cells using TRI Reagent (Molecular Research Center, Cincinnati, USA) according to the manufacturer's instructions. After RNA quantification by Nanodrop 2000, cDNA was synthesized from 1 μg of total RNA using the high-capacity RNA-to-cDNA kit (Thermo Fisher Scientific, Waltham, USA). Real-time PCR was performed using an ABI 7900 PCR device (Thermo Fisher Scientific) and SYBR Green (Biorad, Hercules, USA) with a two-step protocol (94° C. for 15 seconds and 60° C. for 60 seconds). The MET method was used for qPCR data analysis. GAPDH was used as a housekeeping gene control. Experiments were run in triplicates.

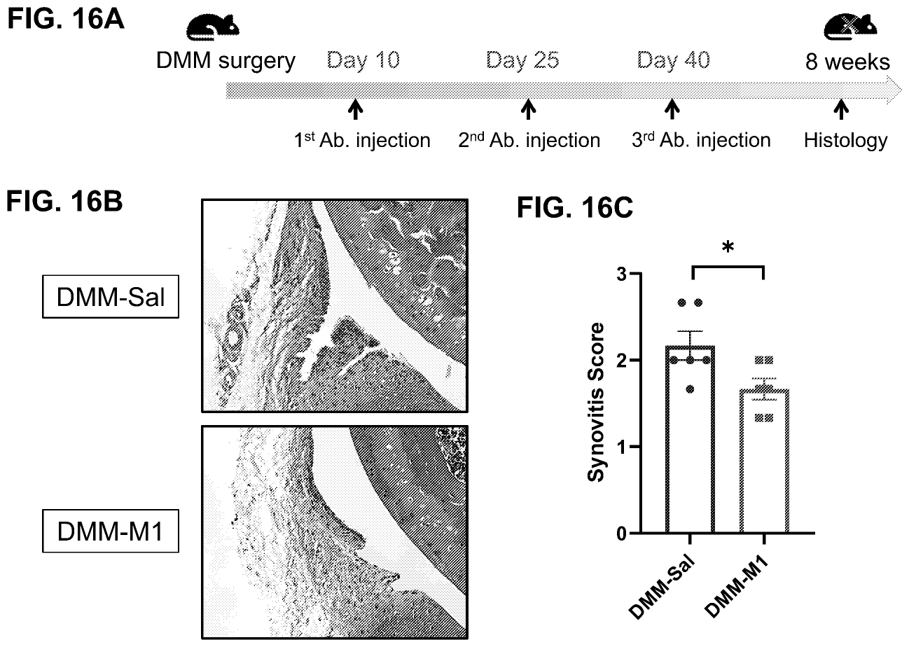

Results. Delayed anti-Cx43 hemichannel antibody (M1 antibody) treatment decreased OA synovitis. It was tested whether delayed administration of anti-Cx43 hemichannel antibody (M1 antibody) could also show effects on mitigating OA progression. Instead of antibody treatment right after surgery, anti-Cx43 hemichannel antibody (M1 antibody) or saline was administered by intraperitoneal injection starting from 10 days after DMM surgery, followed by the second and third injection at 25 days and 40 days post-surgery, respectively. Mice were sacrificed for histology at eight weeks after DMM surgery (FIG. 16A). Synovium hyperplasia was quantified by the synovitis score. Results showed a significant reduction in the degree of synovitis in the anti-Cx43 hemichannel antibody (M1 antibody)-treated group compared with the saline-treated group (FIGS. 16B and C).

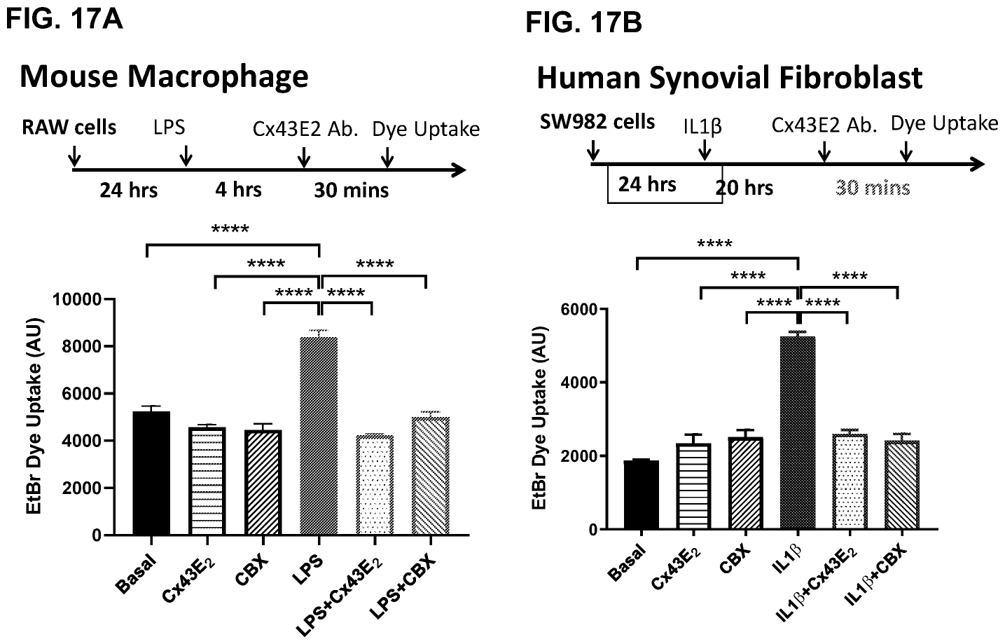

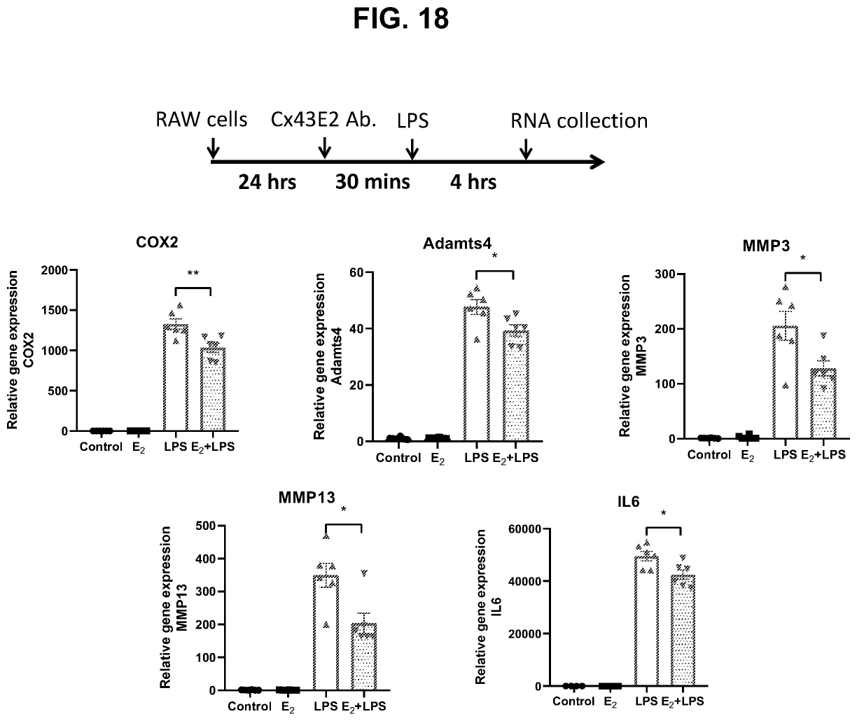

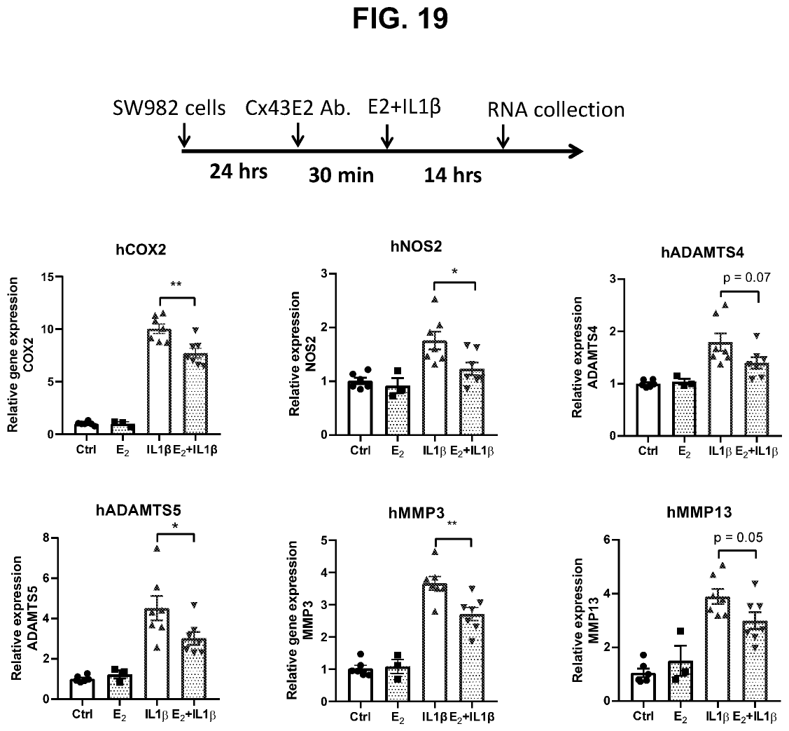

Anti-Cx43 hemichannel antibody (M1 antibody) blocked Cx43 hemichannel opening and inflammatory gene expression in RAW264.7 and SW982 cells. Since the anti-Cx43 hemichannel antibody (M1 antibody) primarily binds to synovium region, including synovium macrophage and fibroblast in vivo, mouse macrophage RAW264.7 cells and human synovial fibroblast SW982 cells were cultured in vitro to study the role of Cx43 hemichannels under inflammation conditions. The activity of Cx43 hemichannels was determined using EtBr dye uptake assay. RAW264.7 cells or SW982 cells were subjected to Lipopolysaccharide (LPS) or Interleukin 1β (IL1β) treatment, respectively. As shown in FIG. 17, LPS and IL1β induced significant increase of Cx43 hemichannel opening, which could be abolished by the Cx43E2 blocking antibody (2 μg/ml) or carbenoxolone (CBX). Next, MMP3/13, Cyclooxygenase 2 (COX2), ADAM metallopeptidase with thrombospondin type 1 motif 4 (Adamts4), and IL6 gene expression was determined with or without Cx43E2 antibody after LPS treatment of RAW264.7 cells (FIG. 18). These inflammatory genes exhibited significant up-regulation induced by LPS, while these changes were markedly decreased in cells pre-treated with Cx43E2 antibody. In addition, MMP3/13, COX2, Adamts4/5, and Nitric Oxide Synthase 2 (NOS2) gene expression was evaluated in IL1β-treated SW982 cells (FIG. 19). Cx43E2 blocking antibody significantly reduced these inflammatory genes expression after IL1β treatment. The above results demonstrate that Cx43 hemichannels play an important role in regulating inflammatory genes expression in macrophage and synovial fibroblast cells.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

1. A method of treating or preventing osteoarthritis in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58 or a variable light chain comprising a sequence having at least 90% identity to a sequence set forth in SEQ ID NO: 60.

2. A method of treating or preventing rheumatoid arthritis in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58 or a variable light chain comprising a sequence having at least 90% identity to a sequence set forth in SEQ ID NO: 60.

3. A method of treating or preventing a tendon disorder or a tendon injury in a subject, the method comprising administering to the subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58 or a variable light chain comprising a sequence having at least 90% identity to a sequence set forth in SEQ ID NO: 60.

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. A method of reducing MMP13 or collagen X levels, the method comprising administering to a subject a therapeutically effective amount of an anti-connexin 43 antibody or a fragment thereof, wherein the antibody or fragment thereof comprises a variable heavy chain comprising a sequence having at least 90% identity to a sequence set forth in SEQ ID NO: 58 or a variable light chain comprising a sequence having at least 90% identity to a sequence set forth in SEQ ID NO: 60.

17. (canceled)

18. (canceled)

19. (canceled)

20. (canceled)

21. (canceled)

22. (canceled)

23. (canceled)

24. (canceled)

25. The method of claim 1, wherein the antibody or fragment thereof comprises a heavy chain immunoglobulin variable region comprising:
a) a complementarity determining region 1 (CDR1) comprising the sequence of SEQ ID NO: 19 or a variant thereof;
b) a complementarity determining region 2 (CDR2) comprising the sequence of SEQ ID NO: 20 or a variant thereof; and
c) a complementarity determining region (CDR3) comprising the sequence of SEQ ID NO: 21 or a variant thereof.

26. The method of claim 1, wherein the antibody or fragment thereof comprises a light chain immunoglobulin variable region comprising:
a) a complementarity determining region 1 (CDR1) comprising the sequence of SEQ ID NO: 31 or a variant thereof;
b) a complementarity determining region 2 (CDR2) comprising the sequence of SEQ ID NO: 32 or a variant thereof; and/or
c) a complementarity determining region 3 (CDR3) comprising the sequence of SEQ ID NO: 33 a variant thereof.

27. The method of claim 25, wherein any one of the CDR1, CDR2 or CDR3 of the heavy chain immunoglobulin variable region comprise at least one amino acid substitution as compared to the parent CDR.

28. The method of claim 27, wherein the at least one amino acid substitution is a cysteine residue to another amino acid or a glycine to another amino acid.

29. The method of claim 25, wherein the antibody or fragment thereof comprises a heavy chain immunoglobulin variable region comprising:
a) a complementarity determining region 1 (CDR1) comprising a sequence having at least 60% identity to a sequence set forth in SEQ ID NO: 19;
b) a complementarity determining region 2 (CDR2) comprising a sequence having at least 60% identity to a sequence set forth in SEQ ID NO: 20; and
c) a complementarity determining region 3 (CDR3) comprising a sequence having at least 60% identity to a sequence set forth in SEQ ID NO: 21; and
wherein the antibody or fragment thereof comprises a light chain immunoglobulin variable region comprising:
a) a complementarity determining region 1 (CDR1) comprising a sequence having at least 60% identity to a sequence set forth in SEQ ID NO: 31;
b) a complementarity determining region 2 (CDR2) comprising a sequence having at least 60% identity to a sequence set forth in SEQ ID NO: 32; and
c) a complementarity determining region 3 (CDR3) comprising a sequence having at least 60% identity to a sequence set forth in SEQ ID NO: 33.

30. (canceled)

31. The method of claim 1, wherein the therapeutically effective amount of the antibody or the fragment thereof is in an expression vector encoding the antibody or the fragment thereof.

32. The method of claim 1, wherein the antibody is administered in a pharmaceutically acceptable composition.

33. The method of claim 1, wherein the antibody is administered systemically.

34. The method of claim 1, wherein the antibody is administered intravenously, intradermally, intratumorally, intramuscularly, intraperitoneally, subcutaneously, or locally.

35. The method of claim 1, wherein the antibody is a humanized antibody.

36. The method of claim 1, further comprising administering at least a second therapeutic agent to the subject.

37. The method of claim 36, wherein the second therapeutic agent is a nonsteroidal anti-inflammatory drug, a glucosamine supplement, a chondroitin supplements, or a joint replacement surgery.

38. (canceled)

39. The method of claim 1, wherein the antibody or fragment thereof further comprises a tag sequence.

40. The method of claim 1, wherein the antibody or fragment thereof inhibits or blocks the opening of a Cx43 hemichannel.

41. The method of claim 1, wherein the fragment thereof is a Fab fragment, an Fab′ fragment or an F(ab′)2 fragment.

42. The method of claim 32, wherein the pharmaceutical composition is lyophilized.

43. (canceled)

44. (canceled)

45. (canceled)

46. (canceled)

47. (canceled)

48. (canceled)
